(19) United States
(12) Patent Application Publication
(10) Pub. No.: US 2026/0234732 A1
(43) Pub. Date: Aug. 13, 2026

(54) DETECTING TUMOR-RELATED INFORMATION BASED ON METHYLATION STATUS OF CELL-FREE NUCLEIC ACID MOLECULES

(71) Applicant: GUARDANT HEALTH, INC., Palo Alto, CA (US)

(72) Inventors: Oscar WESTESSON, Richmond, CA (US); Andrew KENNEDY, San Diego, CA (US); Meromit SINGER, Menlo Park, CA (US)

(21) Appl. No.: 19/637,404

(22) Filed: Apr. 2, 2026

Related U.S. Application Data
(63) Continuation of application No. PCT/US2024/050095, filed on Oct. 4, 2024.
(60) Provisional application No. 63/588,665, filed on Oct. 6, 2023.

(51) Int. Cl. C12Q 1/6886; C12Q 1/6806; C12Q 1/6809; C12Q 1/6869

(52) U.S. Cl. CPC C12Q 1/6886; C12Q 1/6806; C12Q 1/6809; C12Q 1/6869; C12Q 2600/112; C12Q 2600/156

(57) ABSTRACT

In implementations described herein, methylation information is determined with respect to classification regions of a reference genome that are related to the presence of a tumor in a subject. The methylation information can be analyzed using a number of computational techniques to provide metrics related to the presence or absence of a tumor in a given subject.

### CROSS-REFERENCE TO RELATED APPLICATIONS

The present application is a Continuation of International Patent Application No. PCT/US2024/050095, filed Oct. 4, 2024, which claims the benefit of priority of U.S. Provisional Application No. 63/588,665 filed Oct. 6, 2023, which is incorporated by reference herein in its entirety for all purposes.

### BACKGROUND

Cancer is a major cause of disease worldwide. Each year, tens of millions of people are diagnosed with cancer around the world, and more than half eventually die from it. In many countries, cancer ranks the second most common cause of death following cardiovascular diseases. Early detection is associated with improved outcomes for many cancers.

Cancer can be caused by the accumulation of genetic variations within an individual's normal cells, at least some of which result in improperly regulated cell division. Such variations commonly include copy number variations (CNVs), single nucleotide variations (SNVs), gene fusions, insertions and/or deletions (indels), epigenetic variations including 5-methylation of cytosine (5-methylcytosine), and association of DNA with chromatin and transcription factors.

Cancers are often detected by biopsies of tumors followed by analysis of cell markers or DNA extracted from cells. But more recently it has been proposed that cancers can also be detected from cell-free nucleic acids in body fluids, such as blood or urine. Such tests have the advantage that they are noninvasive and can be performed without identifying suspected cancer cells in biopsy. However, such tests are complicated by the fact that the amount of nucleic acids in body fluids is very low and that the nucleic acids that are present are heterogeneous in form (e.g., RNA and DNA, single-stranded and double-stranded, and various states of post-replication modification and association with proteins, such as histones).

Thus, there is a need for improved systems and methods for improved cancer detection using liquid biopsy assays. Therefore, it is an object of the disclosure to provide computer-implemented systems and methods and other processes that have improved capability to classify a sample as containing tumor-derived DNA.

### BRIEF DESCRIPTION OF THE DRAWINGS

The accompanying drawings, which are incorporated in and constitute a part of this specification, illustrate certain implementations, and together with the written description, serve to explain certain principles of the methods, computer readable media, and systems disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 is a diagrammatic representation of an example architecture that analyzes methylation states of nucleic acid molecules derived from samples obtained from different groups of subjects, according to one or more example implementations.

FIG. 2 is a diagrammatic representation of an example environment to identify nucleic acid molecules obtained from different subjects that have overlapping cytosine-guanine regions to determine tumor information for one or more test subjects, according to one or more example implementations.

FIG. 3 is a diagrammatic representation of an example environment to analyze sequence data derived from nucleic acid molecules obtained from a number of groups of subjects according to a number of classification criteria, according to one or more example implementations.

FIG. 4 is a flow diagram of an example process to determine an indication of cancer being present in a test subject by analyzing methylation states of nucleic acid molecules derived from the test subject and derived from one or more additional groups of subjects, according to one or more implementations.

FIG. 5 is a block diagram illustrating components of a machine, in the form of a computer system, that may read and execute instructions from one or more machine-readable media to perform any one or more methodologies described herein, in accordance with one or more example implementations.

FIG. 6 is block diagram illustrating a representative software architecture that may be used in conjunction with one or more hardware architectures described herein, in accordance with one or more example implementations.

FIG. 7 is a graphical representation showing a number of nucleic acid molecules that correspond to a number of genomic regions and that satisfy one or more classification criteria for samples corresponding to subjects in which late-stage cancer is present.

FIG. 8 is a graphical representation showing a number of nucleic acid molecules that correspond to a number of genomic regions and that satisfy one or more classification criteria for samples corresponding to subjects in which stage II cancer is present.

FIG. 9 is a graphical representation showing a number of nucleic acid molecules that correspond to a number of genomic regions and that satisfy one or more classification criteria for samples corresponding to subject in which stage I cancer is present.

### SUMMARY

In one aspect, a method includes accessing one or more data sets indicating (i) first methylation states of individual cytosine nucleotides included in first cytosine-guanine dinucleotides (CpGs) of first deoxyribonucleic acid (DNA) molecule sequence representations in a first dataset derived from first samples obtained from first subjects in which a biological condition is not detected and (ii) second methylation states of individual cytosine nucleotides included in second CpGs of second DNA molecule sequence representations in a second dataset derived from one or more second samples obtained from a second subject, analyzing the second methylation states of the second CpGs with respect to the first methylation states of the first CpGs to determine a subset of the second DNA molecule sequence representations, individual second DNA molecule sequence representations of the subset of the second DNA molecule sequence representations having at least a threshold amount of the second CpGs that correspond to a number of the first CpGs, determining one or more quantitative measures with respect to the subset of the second DNA molecule sequence representations, and determining an indication of the biological condition being present in the second subject based on the one or more quantitative measures. In one or more examples, the biological condition can include one or more types of cancer.

The method may also include accessing an additional data set indicating third methylation states of individual cytosine nucleotides included in third CpGs of third DNA molecule sequence representations in a third dataset derived from one or more third samples obtained from one or more third subjects in which the biological condition is detected, the one or more third samples including one or more tissue samples obtained from the one or more third subjects, and analyzing the second methylation states of the second CpGs with respect to the third methylation states of the third CpGs to determine an additional subset of the second DNA molecule sequence representations, additional individual second DNA molecule sequence representations of the additional subset of the second DNA molecule sequence representations having at least the threshold amount of the second CpGs that correspond to the third CpGs.

The method may also include includes determining a group of the second DNA molecule sequence representations that excludes the subset of the second DNA molecule sequence representations and includes the additional subset of the second DNA molecule sequence representations.

The method may also include where determining the subset of the second DNA molecule sequence representations includes determining an individual second DNA molecule sequence representation having a plurality of CpGs with genomic locations that align with a plurality of additional CpGs at the genomic locations of an individual first DNA molecule sequence representation, determining that a number of the plurality of CpGs of the individual second DNA molecule sequence representation having methylation states of individual cytosines that are the same as additional methylation states of individual additional cytosines of the plurality of additional CpGs of the individual first DNA molecule sequence representation is at least a threshold number, and determining that the individual second DNA molecule sequence representation is to be excluded from the group of the second DNA molecule sequence representations.

The method may also include where determining the group of the second DNA molecule sequence representations that excludes the subset of the second DNA molecule sequence representations and includes the additional subset of the second DNA molecule sequence representations includes determining an individual second DNA molecule sequence representation having a plurality of CpGs with genomic locations that align with a plurality of additional CpGs at the genomic locations of an individual first DNA molecule sequence representation, determining that a number of the plurality of CpGs of the individual second DNA molecule sequence representation having methylation states of individual cytosines that are different from additional methylation states of individual additional cytosines of the plurality of additional CpGs of an individual third DNA molecule sequence representation is at least a threshold number, and determining that the individual second DNA molecule sequence representation is to be included in an additional group of DNA molecule sequence representations that is analyzed with respect to the additional data set.

The method may also include includes determining that the individual second DNA molecule sequence representation has a plurality of CpGs with genomic locations that align with a plurality of additional CpGs at the genomic locations of the individual third DNA molecule sequence representation, determining that a number of the plurality of CpGs of the individual second DNA molecule sequence representation have methylation states of individual cytosines that are the same as additional methylation states of individual additional cytosines of the plurality of additional CpGs of the individual third DNA molecule sequence representation is at least a threshold number, and determining that the individual second DNA molecule sequence representation is to be included in the group of the second DNA molecule sequence representations.

The method may also include includes determining that a number of first DNA molecule sequence representations are matched with an individual second DNA molecule sequence representation and if that number is greater than a threshold then that individual second DNA molecule sequence representation is excluded from the subset of the second DNA molecule sequence representations.

The method may also include where determining that the individual second DNA molecule sequence representation is to be included in the group of the second DNA molecule sequence representations includes determining that a number of the third DNA molecule sequence representations are matched with an individual second molecule sequence representation and if that number is greater than a threshold then that individual second DNA molecule sequence representation is included in the group of the second DNA molecule sequence representations.

The method may also include where determining that the individual second DNA molecule sequence representation has a plurality of CpGs with genomic locations that align with the plurality of additional CpGs at the genomic locations of the individual first DNA molecule sequence representation includes analyzing individual positions of individual CpGs of the individual second DNA molecule sequence representation with respect to individual additional positions of a plurality of additional CpGs of a plurality of first DNA molecule sequence representations to determine one or more DNA molecule sequence representation pairings, where an individual DNA molecule sequence representations pairing includes the second DNA molecule sequence representations and a first DNA molecule sequence representation having at least a threshold number of aligned CpGs, where an aligned CpG corresponds to a position of a reference genome where (i) a CpG of the individual second DNA molecule sequence representation is located and (ii) a CpG of the first DNA molecule sequence representation is located.

The method may also include where analyzing the second methylation states of the second CpGs with respect to the first methylation states of the first CpGs to determine the subset of the second DNA molecule sequence representations includes determining that a methylation state of a CpG of the individual second molecule sequence representation corresponds to an additional methylation state of an additional CpG of a respective first DNA molecule sequence representation for at least a threshold number of aligned CpGs of the individual second molecule sequence representation and the respective first DNA molecule sequence representation.

The method may also include where determining that the individual second DNA molecule sequence representation has a plurality of CpGs with genomic locations that align with the plurality of additional CpGs at the genomic locations of the individual third DNA molecule sequence representation includes analyzing individual positions of individual CpGs of the individual second DNA molecule sequence representation with respect to individual additional positions of a plurality of additional CpGs of a plurality of the third DNA molecule sequence representations to determine one or more DNA molecule sequence representation pairings, where an individual DNA molecule sequence representation pairing includes the individual second DNA molecule sequence representation and a third DNA molecule sequence representation having at least a threshold number of aligned CpGs, where an aligned CpG corresponds to a position of a reference genome where (i) a CpG of the individual second DNA molecule sequence representation is located and (ii) a CpG of the first DNA molecule sequence representation is located.

The method may also include where analyzing the second methylation states of the second CpGs with respect to the third methylation states of the third CpGs to determine the additional subset of the individual second DNA molecule sequence representation includes determining that a methylation state of a CpG of the individual second DNA molecule sequence representation corresponds to an additional methylation state of an additional CpG of a respective third DNA molecule sequence representation for at least at threshold number of aligned CpGs of the individual second DNA molecule sequence representation and the respective third DNA molecule sequence representation.

The method may also include the first DNA molecule sequence representations corresponding to at least one of cell-free DNA molecules derived from one or more plasma samples obtained from the first subjects, or DNA molecules obtained from tissue samples extracted from the first subjects.

The method may also include the second DNA molecule sequence representations correspond to at least one of cell-free DNA molecules derived from one or more plasma samples obtained from the second subject, or DNA molecules obtained from a tissue sample extracted from the second subject.

The method may also include the third DNA molecule sequence representations corresponding to at least one of cell-free DNA molecules derived from one or more plasma samples obtained from the one or more third subjects, or DNA molecules obtained from tissue samples extracted from the one or more third subjects.

The method may also include determining a number of the second DNA molecule sequence representations included in the group of the second DNA molecule sequence representations, and determining an aggregate number of second DNA molecule sequence representations included in the second dataset, where the one or more quantitative measures correspond to a proportion of the number of second DNA molecule sequence representations in relation to the aggregate number of second DNA molecule sequence representations.

The method may also include the one or more quantitative measures including counts of the second DNA molecule sequence representations included in the group of the second DNA molecule sequence representations.

The method may also include a third subject of the one or more third subjects including the second subject.

The method may also include a type of cancer being present in the one or more third subjects and the indication of cancer being present in the second subject indicating that the type of cancer is present in the second subject.

The method may also include a same type of cancer being present in the one or more third subjects.

The method may also include accessing a further data set indicating fourth methylation states of individual cytosine nucleotides included in fourth CpGs of fourth DNA molecule sequence representations derived from one or more fourth samples obtained from one or more fourth subjects in which a tumor corresponding to an additional type of cancer is detected, analyzing the second methylation states of the second CpGs with respect to the fourth methylation states of the fourth CpGs to determine a further subset of the second DNA molecule sequence representations, further individual second DNA molecule sequence representations of the further subset of the second DNA molecule sequence representations having at least the threshold amount of the second CpGs that correspond to the fourth CpGs, determining one or more additional quantitative measures with respect to the subset of the second DNA molecule sequence representations and the further subset of the second DNA molecule sequence representations, and determining an additional indication of the additional type of cancer being present in the second subject based on the one or more additional quantitative measures.

The method may also the one or more fourth samples including at least one of cell-free DNA molecules derived from one or more plasma samples obtained from the one or more fourth subjects, or DNA molecules obtained from tissue samples extracted from the one or more fourth subjects.

The method may also include the indication of cancer being present in the second subject corresponds to tumor fraction, and analyzing the tumor fraction to determine a stage of cancer present in the second subject.

The method may also include at least a portion of the first methylation states indicating first methylated cytosines of the first CpGs, at least a portion of the second methylation states indicate second methylated cytosines of the second CpGs, and at least a portion of the third methylation states indicate third methylated cytosines of the third CpGs.

The method may also include at least a portion of the first methylation states indicating first unmethylated cytosines of the first CpGs, at least a portion of the second methylation states indicate second unmethylated cytosines of the second CpGs, and at least a portion of the third methylation states indicate third unmethylated cytosines of the third CpGs.

The method may also include at least one of the first CpGs, the second CpGs, or the third CpGs being located in a plurality of classification regions, the plurality of classification regions including CpGs having a first methylation state in subjects in which cancer is present and a second methylation state different from the first methylation state in additional subjects in which cancer is not present.

The method may also include determining that at least a threshold number of at least one of the first DNA molecule sequence representations, the second DNA molecule sequence representations, or the third DNA molecule sequence representations correspond to a subset of the plurality of classification regions, where at least one of the first CpGs, the second CpGs, or the third CpGs are located in the subset of the plurality of classification regions.

The method may also include determining that the group of the second DNA molecule sequence representations includes the additional subset of the second DNA molecule sequence representations includes determining that a number of the additional subset of the second DNA molecule sequence representations is at least an additional threshold number. In one aspect, a computing apparatus includes a processor. The computing apparatus also includes a memory storing instructions that, when executed by the processor, configure the apparatus to access one or more data sets indicating (i) first methylation states of individual cytosine nucleotides included in first cytosine-guanine dinucleotides (CpGs) of first deoxyribonucleic acid (DNA) molecule sequence representations in a first dataset derived from first samples obtained from first subjects in which a biological condition is not detected and (ii) second methylation states of individual cytosine nucleotides included in second CpGs of second DNA molecule sequence representations in a second dataset derived from one or more second samples obtained from a second subject, analyze the second methylation states of the second CpGs with respect to the first methylation states of the first CpGs to determine a subset of the second DNA molecule sequence representations, individual second DNA molecule sequence representations of the subset of the second DNA molecule sequence representations having at least a threshold amount of the second CpGs that correspond to a number of the first CpGs, determine one or more quantitative measures with respect to the subset of the second DNA molecule sequence representations, and determine an indication of the biological condition being present in the second subject based on the one or more quantitative measures. In one or more examples, the biological condition can include one or more types of cancer.

The computing apparatus may also include instructions to further configure the apparatus to access an additional data set indicating third methylation states of individual cytosine nucleotides included in third CpGs of third DNA molecule sequence representations in a third dataset derived from one or more third samples obtained from one or more third subjects in which the biological condition is detected, the one or more third samples including one or more tissue samples obtained from the one or more third subjects, and analyze the second methylation states of the second CpGs with respect to the third methylation states of the third CpGs to determine an additional subset of the second DNA molecule sequence representations, additional individual second DNA molecule sequence representations of the additional subset of the second DNA molecule sequence representations having at least the threshold amount of the second CpGs that correspond to the third CpGs.

The computing apparatus may also include instructions to further configure the apparatus to determine a group of the second DNA molecule sequence representations that excludes the subset of the second DNA molecule sequence representations and includes the additional subset of the second DNA molecule sequence representations.

The computing apparatus may also include instructions to further configure the apparatus to determine the subset of the second DNA molecule sequence representations by determining an individual second DNA molecule sequence representation having a plurality of CpGs with genomic locations that align with a plurality of additional CpGs at the genomic locations of an individual first DNA molecule sequence representation, determining that a number of the plurality of CpGs of the individual second DNA molecule sequence representation having methylation states of individual cytosines that are the same as additional methylation states of individual additional cytosines of the plurality of additional CpGs of the individual first DNA molecule sequence representation is at least a threshold number, and determining that the individual second DNA molecule sequence representation is to be excluded from the group of the second DNA molecule sequence representations.

The computing apparatus may also include instructions to further configure the apparatus to determine the group of the second DNA molecule sequence representations that excludes the subset of the second DNA molecule sequence representations and includes the additional subset of the second DNA molecule sequence representations by determining an individual second DNA molecule sequence representation having a plurality of CpGs with genomic locations that align with a plurality of additional CpGs at the genomic locations of an individual first DNA molecule sequence representation, determining that a number of the plurality of CpGs of the individual second DNA molecule sequence representation having methylation states of individual cytosines that are different from additional methylation states of individual additional cytosines of the plurality of additional CpGs of an individual third DNA molecule sequence representation is at least a threshold number, and determining that the individual second DNA molecule sequence representation is to be included in an additional group of DNA molecule sequence representations that is analyzed with respect to the additional data set.

The computing apparatus may also include instructions to further configure the apparatus to determine that the individual second DNA molecule sequence representation has a plurality of CpGs with genomic locations that align with a plurality of additional CpGs at the genomic locations of the individual third DNA molecule sequence representation, determine that a number of the plurality of CpGs of the individual second DNA molecule sequence representation have methylation states of individual cytosines that are the same as additional methylation states of individual additional cytosines of the plurality of additional CpGs of the individual third DNA molecule sequence representation is at least a threshold number, and determine that the individual second DNA molecule sequence representation is to be included in the group of the second DNA molecule sequence representations.

The computing apparatus may also include instructions to further configure the apparatus to determine that a number of first DNA molecule sequence representations are matched with an individual second DNA molecule sequence representation and if that number is greater than a threshold then that individual second DNA molecule sequence representation is excluded from the subset of the second DNA molecule sequence representations.

The computing apparatus may also include instructions to further configure the apparatus to determine that the individual second DNA molecule sequence representation is to be included in the group of the second DNA molecule sequence representations by determining that a number of the third DNA molecule sequence representations are matched with an individual second molecule sequence representation and if that number is greater than a threshold then that individual second DNA molecule sequence representation is included in the group of the second DNA molecule sequence representations.

The computing apparatus may also include instructions to further configure the apparatus to determine that the individual second DNA molecule sequence representation has a plurality of CpGs with genomic locations that align with the plurality of additional CpGs at the genomic locations of the individual first DNA molecule sequence representation by analyzing individual positions of individual CpGs of the individual second DNA molecule sequence representation with respect to individual additional positions of a plurality of additional CpGs of a plurality of first DNA molecule sequence representations to determine one or more DNA molecule sequence representation pairings, where an individual DNA molecule sequence representations pairing includes the second DNA molecule sequence representations and a first DNA molecule sequence representation having at least a threshold number of aligned CpGs, where an aligned CpG corresponds to a position of a reference genome where (i) a CpG of the individual second DNA molecule sequence representation is located and (ii) a CpG of the first DNA molecule sequence representation is located.

The computing apparatus may also include instructions to further configure the apparatus to analyze the second methylation states of the second CpGs with respect to the first methylation states of the first CpGs to determine the subset of the second DNA molecule sequence representations by determining that a methylation state of a CpG of the individual second molecule sequence representation corresponds to an additional methylation state of an additional CpG of a respective first DNA molecule sequence representation for at least a threshold number of aligned CpGs of the individual second molecule sequence representation and the respective first DNA molecule sequence representation.

The computing apparatus may also include instructions to further configure the apparatus to determine that the individual second DNA molecule sequence representation has a plurality of CpGs with genomic locations that align with the plurality of additional CpGs at the genomic locations of the individual third DNA molecule sequence representation by analyzing individual positions of individual CpGs of the individual second DNA molecule sequence representation with respect to individual additional positions of a plurality of additional CpGs of a plurality of the third DNA molecule sequence representations to determine one or more DNA molecule sequence representation pairings, where an individual DNA molecule sequence representation pairing includes the individual second DNA molecule sequence representation and a third DNA molecule sequence representation having at least a threshold number of aligned CpGs, where an aligned CpG corresponds to a position of a reference genome where (i) a CpG of the individual second DNA molecule sequence representation is located and (ii) a CpG of the first DNA molecule sequence representation is located.

The computing apparatus may also include instructions to further configure the apparatus to analyze the second methylation states of the second CpGs with respect to the third methylation states of the third CpGs to determine the additional subset of the individual second DNA molecule sequence representation includes determine that a methylation state of a CpG of the individual second DNA molecule sequence representation corresponds to an additional methylation state of an additional CpG of a respective third DNA molecule sequence representation for at least at threshold number of aligned CpGs of the individual second DNA molecule sequence representation and the respective third DNA molecule sequence representation.

The computing apparatus may also include instructions to further configure the apparatus to determine a number of the second DNA molecule sequence representations included in the group of the second DNA molecule sequence representations, and determine an aggregate number of second DNA molecule sequence representations included in the second dataset, where the one or more quantitative measures correspond to a proportion of the number of second DNA molecule sequence representations in relation to the aggregate number of second DNA molecule sequence representations.

The computing apparatus may also include where the one or more quantitative measures include counts of the second DNA molecule sequence representations included in the group of the second DNA molecule sequence representations.

The computing apparatus may also include where a third subject of the one or more third subjects includes the second subject.

The computing apparatus may also include where a type of cancer is present in the one or more third subjects and the indication of cancer being present in the second subject indicates that the type of cancer is present in the second subject.

The computing apparatus may also include where a same type of cancer is present in the one or more third subjects.

The computing apparatus may also include instructions to further configure the apparatus to access a further data set indicating fourth methylation states of individual cytosine nucleotides included in fourth CpGs of fourth DNA molecule sequence representations derived from one or more fourth samples obtained from one or more fourth subjects in which a tumor corresponding to an additional type of cancer is detected, analyze the second methylation states of the second CpGs with respect to the fourth methylation states of the fourth CpGs to determine a further subset of the second DNA molecule sequence representations, further individual second DNA molecule sequence representations of the further subset of the second DNA molecule sequence representations having at least the threshold amount of the second CpGs that correspond to the fourth CpGs, determine one or more additional quantitative measures with respect to the subset of the second DNA molecule sequence representations and the further subset of the second DNA molecule sequence representations, and determine an additional indication of the additional type of cancer being present in the second subject based on the one or more additional quantitative measures.

The computing apparatus may also include where the indication of cancer being present in the second subject corresponds to tumor fraction, and the method includes analyze the tumor fraction to determine a stage of cancer present in the second subject.

The computing apparatus may also include where at least a portion of the first methylation states indicate first methylated cytosines of the first CpGs, at least a portion of the second methylation states indicate second methylated cytosines of the second CpGs, and at least a portion of the third methylation states indicate third methylated cytosines of the third CpGs.

The computing apparatus may also include where at least a portion of the first methylation states indicate first unmethylated cytosines of the first CpGs, at least a portion of the second methylation states indicate second unmethylated cytosines of the second CpGs, and at least a portion of the third methylation states indicate third unmethylated cytosines of the third CpGs.

The computing apparatus may also include where at least one of the first CpGs, the second CpGs, or the third CpGs are located in a plurality of classification regions, the plurality of classification regions include CpGs having a first methylation state in subjects in which cancer is present and a second methylation state different from the first methylation state in additional subjects in which cancer is not present.

The computing apparatus may also include instructions to further configure the apparatus to determine that at least a threshold number of at least one of the first DNA molecule sequence representations, the second DNA molecule sequence representations, or the third DNA molecule sequence representations correspond to a subset of the plurality of classification regions, where at least one of the first CpGs, the second CpGs, or the third CpGs are located in the subset of the plurality of classification regions.

The computing apparatus may also include instructions to further configure the apparatus to determine that the group of the second DNA molecule sequence representations includes the additional subset of the second DNA molecule sequence representations by determining that a number of the additional subset of the second DNA molecule sequence representations is at least an additional threshold number.

The computing apparatus may also include instructions to further configure the apparatus to obtain a first additional dataset indicating first additional methylation states of individual cytosine nucleotides included in first additional CpGs of first additional DNA molecule sequence representations derived from one or more buffy coat samples obtained from the second subject; and analyze the second methylation states of the second CpGs with respect to the first additional methylation states of the first additional CpGs to determine the subset of the second DNA molecule sequence representations.

The computing apparatus may also include instructions to further configure the apparatus to determine that the second methylation states of a first additional group of the second DNA molecule sequence representations corresponds to the first additional methylation states of at least a portion of the first additional DNA molecule sequence representations; and determine that the first additional group of the second DNA molecule sequence representations is excluded from the subset of the second DNA molecule sequence representations.

The computing apparatus may also include instructions to further configure the apparatus to determine the subset of the second DNA molecule sequence representations by: determining an individual second DNA molecule sequence representation having a plurality of CpGs with genomic locations that align with a plurality of the first additional CpGs at the genomic locations of an individual first additional DNA molecule sequence representation; determining that a number of the plurality of CpGs of the individual second DNA molecule sequence representation having methylation states of individual cytosines that are the same as the first additional methylation states of individual first additional cytosines of the plurality of first additional CpGs of the individual first additional DNA molecule sequence representation is at least a threshold number; and determining that the individual second DNA molecule sequence representation is to be excluded from the subset of the second DNA molecule sequence representations.

The computing apparatus may also include instructions to further configure the apparatus to obtain a second additional dataset indicating second additional methylation states of individual cytosine nucleotides included in second additional CpGs of second additional DNA molecule sequence representations derived from one or more buffy coat samples obtained from at least a portion of the first subjects; and analyze the second methylation states of the second CpGs with respect to the second additional methylation states of the first additional CpGs to determine the subset of the second DNA molecule sequence representations.

The computing apparatus may also include instructions to further configure the apparatus to determine that the second methylation states of a second additional group of the second DNA molecule sequence representations corresponds to the second additional methylation states of at least a portion of the second additional DNA molecule sequence representations; and determine that the second additional group of the second DNA molecule sequence representations is excluded from the subset of the second DNA molecule sequence representations.

The computing apparatus may also include instructions to further configure the apparatus to determine the subset of the second DNA molecule sequence representations by: determining an individual second DNA molecule sequence representation having a plurality of CpGs with genomic locations that align with a plurality of the second additional CpGs at the genomic locations of an individual second additional DNA molecule sequence representation; determining that a number of the plurality of CpGs of the individual second DNA molecule sequence representation having methylation states of individual cytosines that are the same as the second additional methylation states of individual second additional cytosines of the plurality of second additional CpGs of the individual second additional DNA molecule sequence representation is at least a threshold number; and determining that the individual second DNA molecule sequence representation is to be excluded from the subset of the second DNA molecule sequence representations.

In one aspect, a non-transitory computer-readable storage medium, the computer-readable storage medium including instructions that when executed by a computer, cause the computer to access one or more data sets indicating (i) first methylation states of individual cytosine nucleotides included in first cytosine-guanine dinucleotides (CpGs) of first deoxyribonucleic acid (DNA) molecule sequence representations in a first dataset derived from first samples obtained from first subjects in which a biological condition is not detected and (ii) second methylation states of individual cytosine nucleotides included in second CpGs of second DNA molecule sequence representations in a second dataset derived from one or more second samples obtained from a second subject, analyze the second methylation states of the second CpGs with respect to the first methylation states of the first CpGs to determine a subset of the second DNA molecule sequence representations, individual second DNA molecule sequence representations of the subset of the second DNA molecule sequence representations having at least a threshold amount of the second CpGs that correspond to a number of the first CpGs, determine one or more quantitative measures with respect to the subset of the second DNA molecule sequence representations, and determine an indication of the biological condition being present in the second subject based on the one or more quantitative measures. In one or more illustrative examples, the biological condition can include one or more types of cancer.

The computer-readable storage medium may also include instructions to further configure the computer to access an additional data set indicating third methylation states of individual cytosine nucleotides included in third CpGs of third DNA molecule sequence representations in a third dataset derived from one or more third samples obtained from one or more third subjects in which the biological condition is detected, the one or more third samples including one or more tissue samples obtained from the one or more third subjects, and analyze the second methylation states of the second CpGs with respect to the third methylation states of the third CpGs to determine an additional subset of the second DNA molecule sequence representations, additional individual second DNA molecule sequence representations of the additional subset of the second DNA molecule sequence representations having at least the threshold amount of the second CpGs that correspond to the third CpGs.

The computer-readable storage medium may also include instructions to further configure the computer to determine a group of the second DNA molecule sequence representations that excludes the subset of the second DNA molecule sequence representations and includes the additional subset of the second DNA molecule sequence representations.

The computer-readable storage medium may also include instructions to further configure the computer to determine the subset of the second DNA molecule sequence representations by determining an individual second DNA molecule sequence representation having a plurality of CpGs with genomic locations that align with a plurality of additional CpGs at the genomic locations of an individual first DNA molecule sequence representation, determining that a number of the plurality of CpGs of the individual second DNA molecule sequence representation having methylation states of individual cytosines that are the same as additional methylation states of individual additional cytosines of the plurality of additional CpGs of the individual first DNA molecule sequence representation is at least a threshold number, and determining that the individual second DNA molecule sequence representation is to be excluded from the group of the second DNA molecule sequence representations.

The computer-readable storage medium may also include instructions to further configure the computer to determine the group of the second DNA molecule sequence representations that excludes the subset of the second DNA molecule sequence representations and includes the additional subset of the second DNA molecule sequence representations by determining an individual second DNA molecule sequence representation having a plurality of CpGs with genomic locations that align with a plurality of additional CpGs at the genomic locations of an individual first DNA molecule sequence representation, determining that a number of the plurality of CpGs of the individual second DNA molecule sequence representation having methylation states of individual cytosines that are different from additional methylation states of individual additional cytosines of the plurality of additional CpGs of an individual third DNA molecule sequence representation is at least a threshold number, and determining that the individual second DNA molecule sequence representation is to be included in an additional group of DNA molecule sequence representations that is analyzed with respect to the additional data set.

The computer-readable storage medium may also include instructions to further configure the computer to determine that the individual second DNA molecule sequence representation has a plurality of CpGs with genomic locations that align with a plurality of additional CpGs at the genomic locations of the individual third DNA molecule sequence representation, determine that a number of the plurality of CpGs of the individual second DNA molecule sequence representation have methylation states of individual cytosines that are the same as additional methylation states of individual additional cytosines of the plurality of additional CpGs of the individual third DNA molecule sequence representation is at least a threshold number, and determine that the individual second DNA molecule sequence representation is to be included in the group of the second DNA molecule sequence representations.

The computer-readable storage medium may also include instructions to further configure the computer to determine that a number of first DNA molecule sequence representations are matched with an individual second DNA molecule sequence representation and if that number is greater than a threshold then that individual second DNA molecule sequence representation is excluded from the subset of the second DNA molecule sequence representations.

The computer-readable storage medium may also include instructions to further configure the computer to determine that the individual second DNA molecule sequence representation is to be included in the group of the second DNA molecule sequence representations by determining that a number of the third DNA molecule sequence representations are matched with an individual second molecule sequence representation and if that number is greater than a threshold then that individual second DNA molecule sequence representation is included in the group of the second DNA molecule sequence representations.

The computer-readable storage medium may also include instructions to further configure the computer to determine that the individual second DNA molecule sequence representation has a plurality of CpGs with genomic locations that align with the plurality of additional CpGs at the genomic locations of the individual first DNA molecule sequence representation by analyzing individual positions of individual CpGs of the individual second DNA molecule sequence representation with respect to individual additional positions of a plurality of additional CpGs of a plurality of first DNA molecule sequence representations to determine one or more DNA molecule sequence representation pairings, where an individual DNA molecule sequence representations pairing includes the second DNA molecule sequence representations and a first DNA molecule sequence representation having at least a threshold number of aligned CpGs, where an aligned CpG corresponds to a position of a reference genome where (i) a CpG of the individual second DNA molecule sequence representation is located and (ii) a CpG of the first DNA molecule sequence representation is located.

The computer-readable storage medium may also include instructions to further configure the computer to analyze the second methylation states of the second CpGs with respect to the first methylation states of the first CpGs to determine the subset of the second DNA molecule sequence representations includes determine that a methylation state of a CpG of the individual second molecule sequence representation corresponds to an additional methylation state of an additional CpG of a respective first DNA molecule sequence representation for at least a threshold number of aligned CpGs of the individual second molecule sequence representation and the respective first DNA molecule sequence representation.

The computer-readable storage medium may also include instructions to further configure the computer to determine that the individual second DNA molecule sequence representation has a plurality of CpGs with genomic locations that align with the plurality of additional CpGs at the genomic locations of the individual third DNA molecule sequence representation by analyzing individual positions of individual CpGs of the individual second DNA molecule sequence representation with respect to individual additional positions of a plurality of additional CpGs of a plurality of the third DNA molecule sequence representations to determine one or more DNA molecule sequence representation pairings, where an individual DNA molecule sequence representation pairing includes the individual second DNA molecule sequence representation and a third DNA molecule sequence representation having at least a threshold number of aligned CpGs, where an aligned CpG corresponds to a position of a reference genome where (i) a CpG of the individual second DNA molecule sequence representation is located and (ii) a CpG of the first DNA molecule sequence representation is located.

The computer-readable storage medium may also include instructions to further configure the computer to analyze the second methylation states of the second CpGs with respect to the third methylation states of the third CpGs to determine the additional subset of the individual second DNA molecule sequence representation includes determine that a methylation state of a CpG of the individual second DNA molecule sequence representation corresponds to an additional methylation state of an additional CpG of a respective third DNA molecule sequence representation for at least at threshold number of aligned CpGs of the individual second DNA molecule sequence representation and the respective third DNA molecule sequence representation.

The computer-readable storage medium may also include instructions to further configure the computer to determine a number of the second DNA molecule sequence representations included in the group of the second DNA molecule sequence representations, and determine an aggregate number of second DNA molecule sequence representations included in the second dataset, where the one or more quantitative measures correspond to a proportion of the number of second DNA molecule sequence representations in relation to the aggregate number of second DNA molecule sequence representations.

The computer-readable storage medium may also include where the one or more quantitative measures include counts of the second DNA molecule sequence representations included in the group of the second DNA molecule sequence representations.

The computer-readable storage medium may also include where a third subject of the one or more third subjects includes the second subject.

The computer-readable storage medium may also include where a type of cancer is present in the one or more third subjects and the indication of cancer being present in the second subject indicates that the type of cancer is present in the second subject.

The computer-readable storage medium may also include where a same type of cancer is present in the one or more third subjects.

The computer-readable storage medium may also include instructions to further configure the computer to access a further data set indicating fourth methylation states of individual cytosine nucleotides included in fourth CpGs of fourth DNA molecule sequence representations derived from one or more fourth samples obtained from one or more fourth subjects in which a tumor corresponding to an additional type of cancer is detected, analyze the second methylation states of the second CpGs with respect to the fourth methylation states of the fourth CpGs to determine a further subset of the second DNA molecule sequence representations, further individual second DNA molecule sequence representations of the further subset of the second DNA molecule sequence representations having at least the threshold amount of the second CpGs that correspond to the fourth CpGs, determine one or more additional quantitative measures with respect to the subset of the second DNA molecule sequence representations and the further subset of the second DNA molecule sequence representations, and determine an additional indication of the additional type of cancer being present in the second subject based on the one or more additional quantitative measures.

The computer-readable storage medium may also include where the indication of cancer being present in the second subject corresponds to tumor fraction, and the method includes analyze the tumor fraction to determine a stage of cancer present in the second subject.

The computer-readable storage medium may also include where at least a portion of the first methylation states indicate first methylated cytosines of the first CpGs, at least a portion of the second methylation states indicate second methylated cytosines of the second CpGs, and at least a portion of the third methylation states indicate third methylated cytosines of the third CpGs.

The computer-readable storage medium may also include where at least a portion of the first methylation states indicate first unmethylated cytosines of the first CpGs, at least a portion of the second methylation states indicate second unmethylated cytosines of the second CpGs, and at least a portion of the third methylation states indicate third unmethylated cytosines of the third CpGs.

The computer-readable storage medium may also include where at least one of the first CpGs, the second CpGs, or the third CpGs are located in a plurality of classification regions, the plurality of classification regions include CpGs having a first methylation state in subjects in which cancer is present and a second methylation state different from the first methylation state in additional subjects in which cancer is not present.

The computer-readable storage medium may also include instructions to further configure the computer to determine that at least a threshold number of at least one of the first DNA molecule sequence representations, the second DNA molecule sequence representations, or the third DNA molecule sequence representations correspond to a subset of the plurality of classification regions, where at least one of the first CpGs, the second CpGs, or the third CpGs are located in the subset of the plurality of classification regions.

The computer-readable storage medium may also include instructions to further configure the computer to determine that the group of the second DNA molecule sequence representations includes the additional subset of the second DNA molecule sequence representations by determining that a number of the additional subset of the second DNA molecule sequence representations is at least an additional threshold number.

The computer-readable storage medium may also include instructions to further configure the computer to obtain a first additional dataset indicating first additional methylation states of individual cytosine nucleotides included in first additional CpGs of first additional DNA molecule sequence representations derived from one or more buffy coat samples obtained from the second subject; and analyze the second methylation states of the second CpGs with respect to the first additional methylation states of the first additional CpGs to determine the subset of the second DNA molecule sequence representations.

The computer-readable storage medium may also include instructions to further configure the computer to determine that the second methylation states of a first additional group of the second DNA molecule sequence representations corresponds to the first additional methylation states of at least a portion of the first additional DNA molecule sequence representations; and determine that the first additional group of the second DNA molecule sequence representations is excluded from the subset of the second DNA molecule sequence representations.

The computer-readable storage medium may also include instructions to further configure the computer to determine the subset of the second DNA molecule sequence representations by: determining an individual second DNA molecule sequence representation having a plurality of CpGs with genomic locations that align with a plurality of the first additional CpGs at the genomic locations of an individual first additional DNA molecule sequence representation; determining that a number of the plurality of CpGs of the individual second DNA molecule sequence representation having methylation states of individual cytosines that are the same as the first additional methylation states of individual first additional cytosines of the plurality of first additional CpGs of the individual first additional DNA molecule sequence representation is at least a threshold number; and determining that the individual second DNA molecule sequence representation is to be excluded from the subset of the second DNA molecule sequence representations.

The computer-readable storage medium may also include instructions to further configure the computer to obtain a second additional dataset indicating second additional methylation states of individual cytosine nucleotides included in second additional CpGs of second additional DNA molecule sequence representations derived from one or more buffy coat samples obtained from at least a portion of the first subjects; and analyze the second methylation states of the second CpGs with respect to the second additional methylation states of the first additional CpGs to determine the subset of the second DNA molecule sequence representations.

The computer-readable storage medium may also include instructions to further configure the computer to determine that the second methylation states of a second additional group of the second DNA molecule sequence representations corresponds to the second additional methylation states of at least a portion of the second additional DNA molecule sequence representations; and determine that the second additional group of the second DNA molecule sequence representations is excluded from the subset of the second DNA molecule sequence representations.

The computer-readable storage medium may also include instructions to further configure the computer to determine the subset of the second DNA molecule sequence representations by: determining an individual second DNA molecule sequence representation having a plurality of CpGs with genomic locations that align with a plurality of the second additional CpGs at the genomic locations of an individual second additional DNA molecule sequence representation; determining that a number of the plurality of CpGs of the individual second DNA molecule sequence representation having methylation states of individual cytosines that are the same as the second additional methylation states of individual second additional cytosines of the plurality of second additional CpGs of the individual second additional DNA molecule sequence representation is at least a threshold number; and determining that the individual second DNA molecule sequence representation is to be excluded from the subset of the second DNA molecule sequence representations.

Other technical features may be readily apparent to one skilled in the art from the following figures, descriptions, and claims.

### Definitions

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons of ordinary skill in the art upon reading this disclosure and so forth.

It is also to be understood that the terminology used herein is for the purpose of describing particular implementations only and is not intended to be limiting. Further, unless defined otherwise, all technical and scientific terms used herein have the same meaning as commonly understood by one of ordinary skill in the art to which this disclosure pertains. In describing and claiming the methods, computer readable media, and systems, the following terminology, and grammatical variants thereof, will be used in accordance with the definitions set forth below.

About: As used herein, “about” or “approximately” as applied to one or more values or elements of interest, refers to a value or element that is similar to a stated reference value or element. In certain implementations, the term “about” or “approximately” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Administer: As used herein, “administer” or “administering” a therapeutic agent (e.g., an immunological therapeutic agent) to a subject means to give, apply or bring the composition into contact with the subject. Administration can be accomplished by any of a number of routes, including, for example, topical, oral, subcutaneous, intramuscular, intraperitoneal, intravenous, intrathecal and intradermal.

Adapter. As used herein, “adapter” refers to a short nucleic acid (e.g., less than about 500 nucleotides, less than about 100 nucleotides, or less than about 50 nucleotides in length) that can be at least partially double-stranded and used to link to either or both ends of a given sample nucleic acid molecule. Adapters can include nucleic acid primer binding sites to permit amplification of a nucleic acid molecule flanked by adapters at both ends, and/or a sequencing primer binding site, including primer binding sites for sequencing applications, such as various next-generation sequencing (NGS) applications. Adapters can also include binding sites for capture probes, such as an oligonucleotide attached to a flow cell support or the like. Adapters can also include a nucleic acid tag as described herein. Nucleic acid tags can be positioned relative to amplification primer and sequencing primer binding sites, such that a nucleic acid tag is included in amplicons and sequence reads of a given nucleic acid molecule. The same or different adapters can be linked to the respective ends of a nucleic acid molecule. In some implementations, the same adapter is linked to the respective ends of the nucleic acid molecule except that the nucleic acid tag differs. In some implementations, the adapter is a Y-shaped adapter in which one end is blunt ended or tailed as described herein, for joining to a nucleic acid molecule, which is also blunt ended or tailed with one or more complementary nucleotides. In still other example implementations, an adapter is a bell-shaped adapter that includes a blunt or tailed end for joining to a nucleic acid molecule to be analyzed. Other examples of adapters include T-tailed and C-tailed adapters.

Alignment: As used herein, “alignment” or “align” refers to determining whether at least two sequence representations have at least a threshold amount of homology. In one or more examples, the threshold amount of homology can be at least about 90%, at least about 91%, at least about 92%, at least about 93%, at least about 94%, at least about 95%, at least about 96%, at least about 97%, at least about 98%, at least about 99%, at least about 99.5%, or at least about 99.9%. In situations where two sequence representations have at least the threshold amount of homology, the two sequence representations can be referred to as being “aligned.”

Amplify. As used herein, “amplify” or “amplification” in the context of nucleic acids refers to the production of multiple copies of a polynucleotide, or a portion of the polynucleotide, starting from a small amount of the polynucleotide (e.g., a single polynucleotide molecule), where the amplification products or amplicons are generally detectable. Amplification of polynucleotides encompasses a variety of chemical and enzymatic processes.

Barcode: As used herein, “barcode” or “molecular barcode” in the context of nucleic acids refers to a nucleic acid molecule comprising a sequence that can serve as a molecular identifier. For example, individual “barcode” sequences can be added to each DNA fragment during next-generation sequencing (NGS) library preparation so that each read can be identified and sorted before the final data analysis.

Cancer Type: As used herein, “cancer type” refers to a type or subtype of cancer defined, e.g., by histopathology. Cancer type can be defined by any conventional criterion, such as on the basis of occurrence in a given tissue (e.g., blood cancers, central nervous system (CNS), brain cancers, lung cancers (small cell and non-small cell), skin cancers, nose cancers, throat cancers, liver cancers, bone cancers, lymphomas, pancreatic cancers, bowel cancers, rectal cancers, thyroid cancers, bladder cancers, kidney cancers, mouth cancers, stomach cancers, breast cancers, prostate cancers, ovarian cancers, lung cancers, intestinal cancers, soft tissue cancers, neuroendocrine cancers, gastroesophageal cancers, head and neck cancers, gynecological cancers, colorectal cancers, urothelial cancers, solid state cancers, heterogeneous cancers, homogenous cancers), unknown primary origin and the like, and/or of the same cell lineage (e.g., carcinoma, sarcoma, lymphoma, cholangiocarcinoma, leukemia, mesothelioma, melanoma, or glioblastoma) and/or cancers exhibiting cancer markers, such as Her2, CA15-3, CA19-9, CA-125, CEA, AFP, PSA, HCG, hormone receptor and NMP-22. Cancers can also be classified by stage (e.g., stage 1, 2, 3, or 4) and whether of primary or secondary origin.

Carrier Signal: As used herein, “carrier signal” refers to any intangible medium that is capable of storing, encoding, or carrying transitory or non-transitory instructions for execution by a machine, and includes digital or analog communications signals or other intangible medium to facilitate communication of such instructions. Instructions may be transmitted or received over a network using a transitory or non-transitory transmission medium via a network interface device and using any one of a number of data transfer protocols.

Cell-Free Nucleic Acid: As used herein, “cell-free nucleic acid” refers to nucleic acids not contained within or otherwise bound to a cell or, in some implementations, nucleic acids remaining in a sample following the removal of intact cells. Cell-free nucleic acids can include, for example, all non-encapsulated nucleic acids sourced from a bodily fluid (e.g., blood, plasma, serum, urine, cerebrospinal fluid (CSF), etc.) from a subject. Cell-free nucleic acids include DNA (cfDNA), RNA (cfRNA), and hybrids thereof, including genomic DNA, mitochondrial DNA, circulating DNA, siRNA, miRNA, circulating RNA (CRNA), tRNA, rRNA, small nucleolar RNA (snoRNA), Piwi-interacting RNA (piRNA), long non-coding RNA (long ncRNA), and/or fragments of any of these. Cell-free nucleic acids can be double-stranded, single-stranded, or a hybrid thereof. A cell-free nucleic acid can be released into bodily fluid through secretion or cell death processes, e.g., cellular necrosis, apoptosis, or the like. Some cell-free nucleic acids are released into bodily fluid from cancer cells, e.g., circulating tumor DNA (ctDNA). Others are released from healthy cells. CtDNA can be non-encapsulated tumor-derived fragmented DNA. A cell-free nucleic acid can have one or more epigenetic modifications, for example, a cell-free nucleic acid can be acetylated, 5-methylated, ubiquitylated, phosphorylated, sumoylated, ribosylated, and/or citrullinated.

Cellular Nucleic Acids: As used herein, “cellular nucleic acids” means nucleic acids that are disposed within one or more cells at least at the point a sample is taken or collected from a subject, even if those nucleic acids are subsequently removed as part of a given analytical process.

Classification Region: As used herein, “classification region” refers to a genomic region that may show sequence-independent changes in neoplastic cells (e.g., tumor cells and cancer cells) or that may show sequence-independent changes in cfDNA from subjects having cancer relative to cfDNA from subjects in which cancer is not present. Examples of sequence-independent changes include, but are not limited to, changes in methylation rate (increases or decreases), nucleosome distribution, CTCF binding, transcription start sites, and regulatory protein binding regions. In one or more examples, sequence-independent changes in a classification region can indicate the presence of a single form of cancer in a subject. In one or more additional examples, sequence-independent changes in a classification region can correspond to the presence of multiple forms in a subject. The classification region can be enriched by one or more probes. In addition, the classification region can be defined by a pair of primer binding sites. Further, the classification region can be defined by a predetermined beginning genomic locus and a predetermined ending genomic locus. The classification region can include from about 25 nucleotides to about 250 nucleotides, from about 50 nucleotides to about 200 nucleotides, or from about 75 nucleotides to about 150 nucleotides. For instance, classification region can be a differentially methylated region. “Differentially methylated region” or “DMR” refers to a region of DNA having a detectably different degree of methylation in at least one cell or tissue type relative to the degree of methylation in the same region of DNA from at least one other cell or tissue type; or having a detectably different degree of methylation in at least one cell or tissue type obtained from a subject having a disease or disorder relative to the degree of methylation in the same region of DNA in the same cell or tissue type obtained from a healthy subject. In some embodiments, a differentially methylated region has a detectably higher degree of methylation in at least one cell or tissue type relative to the degree of methylation in the same region of DNA from at least one other cell or tissue type that contribute to cfDNA in healthy individuals, or from the same cell or tissue type from a healthy subject. In some embodiments, a differentially methylated region has a detectably lower degree of methylation in at least one cell or tissue type relative to the degree of methylation in the same region of DNA from at least one other cell or tissue type, such as other immune cell types and/or cell types that contribute to cfDNA in healthy individuals, or from the same cell or tissue type from a healthy subject.

Communications Network: As used herein, “communications network” refers to one or more portions of a network that may be an ad hoc network, an intranet, an extranet, a virtual private network (VPN), a local area network (LAN), a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network or a portion of a network may include a wireless or cellular network and the coupling may be a Code Division Multiple Access (CDMA) connection, a Global System for Mobile communications (GSM) connection, or other type of cellular or wireless coupling. In this example, the coupling may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard setting organizations, other long range protocols, or other data transfer technology.

Confidence Interval: As used herein, “confidence interval” means a range of values so defined that there is a specified probability that the value of a given parameter lies within that range of values.

CpG: As used herein, “CpG” or “cytosine-guanine dinucleotide” refers to a cytosine-phosphate-guanine site within a nucleic acid molecule sequence such that a cytosine molecule is followed by a guanine molecule in a 5′→3′ direction of the nucleic acid molecule sequence.

Deoxyribonucleic Acid or Ribonucleic Acid: As used herein, “deoxyribonucleic acid” or “DNA” refers to a natural or modified nucleotide which has a hydrogen group at the 2′-position of the sugar moiety. DNA can include a chain of nucleotides comprising four types of nucleotide bases: adenine (A), thymine (T), cytosine (C), and guanine (G). As used herein, “ribonucleic acid” or “RNA” refers to a natural or modified nucleotide which has a hydroxyl group at the 2′-position of the sugar moiety. RNA can include a chain of nucleotides comprising four types of nucleotides: A, uracil (U), G, and C. As used herein, the term “nucleotide” refers to a natural nucleotide or a modified nucleotide. Certain pairs of nucleotides specifically bind to one another in a complementary fashion (called complementary base pairing). In DNA, adenine (A) pairs with thymine (T) and cytosine (C) pairs with guanine (G). In RNA, adenine (A) pairs with uracil (U) and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data”, “nucleic acid sequencing information”, “sequence information”, “sequence representation”, “nucleic acid sequence”, “nucleotide sequence”, “genomic sequence”, “genetic sequence”, “fragment sequence”, “sequencing read”, or “nucleic acid sequencing read” denotes any information or data that is indicative of the order and identity of the nucleotide bases (e.g., adenine, guanine, cytosine, and thymine or uracil) in a molecule (e.g., a whole genome, whole transcriptome, exome, oligonucleotide, polynucleotide, or fragment) of a nucleic acid such as DNA or RNA. It should be understood that the present teachings contemplate sequence information obtained using all available varieties of techniques, platforms or technologies, including, but not limited to capillary electrophoresis, microarrays, ligation-based systems, polymerase-based systems, hybridization-based systems, direct or indirect nucleotide identification systems, pyrosequencing, ion- or pH-based detection systems, and electronic signature-based systems.

Differentially Methylated Region: As used herein, differentially methylated region” refers to a region of DNA having a detectably different degree of methylation in at least one cell or tissue type relative to the degree of methylation in the same region of DNA from at least one other cell or tissue type; or having a detectably different degree of methylation in at least one cell or tissue type obtained from a subject having a disease or disorder relative to the degree of methylation in the same region of DNA in the same cell or tissue type obtained from a healthy subject. In some embodiments, a differentially methylated region has a detectably higher degree of methylation in at least one cell or tissue type, such as at least one immune cell type, relative to the degree of methylation in the same region of DNA from at least one other cell or tissue type, such as other immune cell types and/or cell types that contribute to cfDNA in healthy individuals, or from the same cell or tissue type from a healthy subject. In some embodiments, a differentially methylated region has a detectably lower degree of methylation in at least one cell or tissue type, such as at least one immune cell type, relative to the degree of methylation in the same region of DNA from at least one other cell or tissue type, such as other immune cell types and/or cell types that contribute to cfDNA in healthy individuals, or from the same cell or tissue type from a healthy subject.

Driver Mutation: As used herein, “driver mutation” means a mutation that drives cancer progression.

Epigenetic Target Regions: As used herein, “epigenetic target regions” refers to target regions that may show sequence-independent differences in different cell or tissue types (e.g., different types of immune cells) or in neoplastic cells (e.g., tumor cells and cancer cells) relative to normal cells; or that may show sequence-independent differences (i.e., in which there is no change to the nucleotide sequence, e.g., differences in methylation, nucleosome distribution, or other epigenetic features) in DNA, such as cfDNA, from different cell types or from subjects having cancer relative to DNA, such as cfDNA, from healthy subjects, or in cfDNA originating from different cell or tissue types that ordinarily do not substantially contribute to cfDNA (e.g., immune, lung, colon, etc.) relative to background cfDNA (e.g., cfDNA that originated from hematopoietic cells). Examples of sequence-independent changes include, but are not limited to, changes in methylation (increases or decreases), nucleosome distribution, cfDNA fragmentation patterns, CCCTC-binding factor (“CTCF”) binding, transcription start sites (e.g., with respect to any one of more of binding of RNA polymerase components, binding of regulatory proteins, fragmentation characteristics, and nucleosomal distribution), and regulatory protein binding regions. Epigenetic target region sets thus include, but are not limited to, hypermethylation variable target region sets, hypomethylation variable target region sets, and fragmentation variable target region sets, such as CTCF binding sites and transcription start sites. For present purposes, loci susceptible to neoplasia-, tumor-, or cancer-associated focal amplifications and/or gene fusions may also be included in an epigenetic target region set because detection of a change in copy number by sequencing or a fused sequence that maps to more than one locus in a reference genome tends to be more similar to detection of exemplary epigenetic changes discussed above than detection of nucleotide substitutions, insertions, or deletions, e.g., in that the focal amplifications and/or gene fusions can be detected at a relatively shallow depth of sequencing because their detection does not depend on the accuracy of base calls at one or a few individual positions. An epigenetic target region set is a set of epigenetic target regions.

Immunotherapy: As used herein, “immunotherapy” refers to treatment with one or more agents that act to stimulate the immune system so as to kill or at least to inhibit growth of cancer cells, and preferably to reduce further growth of the cancer, reduce the size of the cancer and/or eliminate the cancer. Some such agents bind to a target present on cancer cells; some bind to a target present on immune cells and not on cancer cells; some bind to a target present on both cancer cells and immune cells. Such agents include, but are not limited to, checkpoint inhibitors and/or antibodies. Checkpoint inhibitors are inhibitors of pathways of the immune system that maintain self-tolerance and modulate the duration and amplitude of physiological immune responses in peripheral tissues to minimize collateral tissue damage (see, e.g., Pardoll, Nature Reviews Cancer 12, 252-264 (2012)). Example agents include antibodies against any of PD-1, PD-2, PD-L1, PD-L2, CTLA-40, OX40, B7.1, B7He, LAG3, CD137, KIR, CCR5, CD27, or CD40. Other example agents include proinflammatory cytokines, such as IL-1B, IL-6, and TNF-α. Other example agents are T-cells activated against a tumor, such as T-cells activated by expressing a chimeric antigen targeting a tumor antigen recognized by the T-cell.

Indel: As used herein, “indel” refers to a mutation that involves the insertion or deletion of nucleotides in the genome of a subject.

Limit of Detection (LoD): As used herein, “limit of detection” means the smallest amount of a substance (e.g., a nucleic acid) in a sample that can be measured by a given assay or analytical approach.

Machine-Readable Medium: As used herein, “machine-readable medium” refers to a component, device, or other tangible media able to store instructions and data temporarily or permanently and may include, but is not limited to, random-access memory (RAM), read-only memory (ROM), buffer memory, flash memory, optical media, magnetic media, cache memory, other types of storage (e.g., erasable programmable read-only memory (EEPROM)) and/or any suitable combination thereof. The term “machine-readable medium” may be taken to include a single medium or multiple media (e.g., a centralized or distributed database, or associated caches and servers) able to store instructions. The term “machine-readable medium” shall also be taken to include any medium, or combination of multiple media, that is capable of storing instructions (e.g., code) for execution by a machine and that when executed by one or more processors of the machine, cause the machine to perform any one or more of the methodologies described herein. Accordingly, a “machine-readable medium” refers to a single storage apparatus or device, as well as “cloud-based” storage systems or storage networks that include multiple storage apparatus or devices. The term “machine-readable medium” excludes signals per se.

Maximum MAF: As used herein, “maximum MAF” or “max MAF” refers to the maximum MAF (mutant allele fraction) of all somatic variants in a sample.

Methylation: As used herein, “methylation” or “DNA methylation” refers to addition of a methyl group to a nucleotide base in a nucleic acid molecule. In some embodiments, methylation refers to addition of a methyl group to a cytosine at a CpG site. In some embodiments, DNA methylation refers to addition of a methyl group to adenine, such as in N⁶-methyladenine. In some embodiments, DNA methylation is 5-methylation (modification of the 5th carbon of the 6-carbon ring of cytosine). In some embodiments, 5-methylation refers to addition of a methyl group to the 5C position of the cytosine to create 5-methylcytosine (5mC). In some embodiments, methylation comprises a derivative of 5mC. Derivatives of 5mC include, but are not limited to, 5-hydroxymethylcytosine (5-hmC), 5-formylcytosine (5-fC), and 5-caryboxylcytosine (5-caC). In some embodiments, DNA methylation is 3C methylation (modification of the 3rd carbon of the 6-carbon ring of cytosine). In some embodiments, 3C methylation comprises addition of a methyl group to the 3C position of the cytosine to generate 3-methylcytosine (3mC). Methylation can also occur at non CpG sites, for example, methylation can occur at a CpA, CpT, or CpC site. DNA methylation can change the activity of methylated DNA region. For example, when DNA in a promoter region is methylated, transcription of the gene may be repressed. DNA methylation is critical for normal development and abnormality in methylation may disrupt epigenetic regulation. The disruption, e.g., repression, in epigenetic regulation may cause diseases, such as cancer. Promoter methylation in DNA may be indicative of cancer.

Methylation rate: As used herein, “methylation rate” refers to the probability, likelihood, or percentage that a given base (for example: cytosine residue in a CpG) is methylated on a DNA molecule at a particular genomic region analyzed in the sample. In some embodiments, the methylation rate may be applied to a defined region that comprises one or more potentially methylated bases. In some embodiments, the methylation rate refers to the percentage of CpG residues methylated in a DNA molecule. In some embodiments, the methylation rate refers to the percentage of CpG residues methylated in molecules aligned to particular genomic position or genomic region. Methylation rate can be measured by a variety of methods including, but not limited to, either using bisulfite sequencing (any single base resolution like TAPS, EM-SEQ, etc.) or using partitioning (DNA molecule resolution). Methylation rate can be measured in different ways. One estimation can be by counting how many DNA fragments end up in each methylation dependent partition or by counting the number of converted CpGs per fragment in the case of bisulfite sequencing or any other base-level resolution sequencing methods. In addition, in the case of methylation dependent partitioning, the rate calculation can be normalized using a set of predefined regions with known methylation state (i.e., positive control regions and/or negative control regions) or spiked-in synthetic DNA with known methylation state, deriving rate-parametrized partition distributions and estimating the rate using a maximum likelihood approach. In one or more examples, the methylation rate can be determined by determining an abundance of sequencing reads that correspond to a portion of a genomic region. The portion of the genomic region can include a number of genomic locations of the genomic region for which at least a threshold number of sequencing reads overlap.

Methylation Status: As used herein, “methylation status” or “methylation state” can refer to the presence or absence of methyl group on a DNA base (e.g., cytosine) at a particular genomic position in a nucleic acid molecule. It can also refer to the degree of methylation in a nucleic acid sequence (e.g., highly methylated, low methylated, intermediately methylated or unmethylated nucleic acid molecules). The methylation status can also refer to the number of nucleotides methylated in a particular nucleic acid molecule.

Mutant Allele Fraction: As used herein, “mutant allele fraction”, “mutation dose,” or “MAF” refers to the fraction of nucleic acid molecules harboring an allelic alteration or mutation at a given genomic position in a given sample. MAF is generally expressed as a fraction or a percentage. For example, an MAF can be less than about 0.5, 0.1, 0.05, or 0.01 (i.e., less than about 50%, 10%, 5%, or 1%) of all somatic variants or alleles present at a given locus.

Mutation: As used herein, “mutation” refers to a variation from a known reference sequence and includes mutations such as, for example, single nucleotide variants (SNVs), copy number variants or variations (CNVs)/aberrations, insertions or deletions (indels), gene fusions, transversions, translocations, frame shifts, duplications, repeat expansions, and epigenetic variants. A mutation can be a germline or somatic mutation. In some examples, a reference sequence for purposes of comparison is a wildtype genomic sequence of the species of the subject providing a test sample, typically the human genome.

Mutation Count: As used herein, “mutation count” or “mutational count” refers to the number of somatic mutations in a whole genome or exome or targeted regions of a nucleic acid sample.

Neoplasm: As used herein, the terms “neoplasm” and “tumor” are used interchangeably. They refer to abnormal growth of cells in a subject. A neoplasm or tumor can be benign, potentially malignant, or malignant. A malignant tumor is referred to as a cancer or a cancerous tumor.

Next Generation Sequencing: As used herein, “next generation sequencing” or “NGS” refers to sequencing technologies having increased throughput as compared to traditional Sanger- and capillary electrophoresis-based approaches, for example, with the ability to generate hundreds of thousands of relatively small sequencing reads at a time. Some examples of next generation sequencing techniques include, but are not limited to, sequencing by synthesis, sequencing by ligation, and sequencing by hybridization.

Nucleic Acid Tag: As used herein, “nucleic acid tag” refers to a short nucleic acid (e.g., less than about 500 nucleotides, about 100 nucleotides, about 50 nucleotides, or about 10 nucleotides in length), used to distinguish nucleic acids from different samples (e.g., representing a sample index), or different nucleic acid molecules in the same sample (e.g., representing a molecular barcode), of different types, or which have undergone different processing. The nucleic acid tag comprises a predetermined, fixed, non-random, random or semi-random oligonucleotide sequence. Such nucleic acid tags may be used to label different nucleic acid molecules or different nucleic acid samples or sub-samples. Nucleic acid tags can be single-stranded, double-stranded, or at least partially double-stranded. Nucleic acid tags optionally have the same length or varied lengths. Nucleic acid tags can also include double-stranded molecules having one or more blunt-ends, include 5′ or 3′ single-stranded regions (e.g., an overhang), and/or include one or more other single-stranded regions at other locations within a given molecule. Nucleic acid tags can be attached to one end or to both ends of the other nucleic acids (e.g., sample nucleic acids to be amplified and/or sequenced). Nucleic acid tags can be decoded to reveal information such as the sample of origin, form, or processing of a given nucleic acid. For example, nucleic acid tags can also be used to enable pooling and/or parallel processing of multiple samples comprising nucleic acids bearing different molecular barcodes and/or sample indexes in which the nucleic acids are subsequently being deconvolved by detecting (e.g., reading) the nucleic acid tags. Nucleic acid tags can also be referred to as identifiers (e.g., molecular identifier, sample identifier). Additionally, or alternatively, nucleic acid tags can be used as molecular identifiers (e.g., to distinguish between different molecules or amplicons of different parent molecules in the same sample or sub-sample). This includes, for example, uniquely tagging different nucleic acid molecules in a given sample, or non-uniquely tagging such molecules. In the case of non-unique tagging applications, a limited number of tags (i.e., molecular barcodes) may be used to tag each nucleic acid molecule such that different molecules can be distinguished based on their endogenous sequence information (for example, start and/or stop positions where they map to a selected reference sequence, a sub-sequence of one or both ends of a sequence, and/or length of a sequence) in combination with at least one molecular barcode. A sufficient number of different molecular barcodes are used such that there is a low probability (e.g., less than about a 10%, less than about a 5%, less than about a 1%, or less than about a 0.1% chance) that any two molecules may have the same endogenous sequence information (e.g., start and/or stop positions, subsequences of one or both ends of a sequence, and/or lengths) and also have the same molecular barcode.

Polynucleotide: As used herein, “polynucleotide”, “nucleic acid”, “nucleic acid molecule”, “polynucleotide molecule”, or “oligonucleotide” refers to a linear polymer of nucleosides (including deoxyribonucleosides, ribonucleosides, or analogs thereof) joined by internucleosidic linkages. A polynucleotide can comprise at least three nucleosides. Oligonucleotides often range in size from a few monomeric units, e.g., 3-4, to hundreds of monomeric units. Whenever a polynucleotide is represented by a sequence of letters, such as “AGCTG,” it will be understood that the nucleotides are in 5′→3′ order from left to right and that in the case of DNA, “A” denotes deoxyadenosine, “C” denotes deoxycytidine, “G” denotes deoxyguanosine, and “T” denotes deoxythymidine, unless otherwise noted. The letters A, C, G, and T may be used to refer to the bases themselves, to nucleosides, or to nucleotides comprising the bases, as is standard in the art.

Probe: As used herein, “probe” refers to a polynucleotide comprising a functionality. The functionality can be a detectable label (fluorescent), a binding moiety (biotin), or a solid support (a magnetically attractable particle or a chip). Probes can include single-stranded DNA/RNA A polynucleotides or double e stranded DNA polynucleotides that hybridize to target nucleic acid sequences (e.g., SureSelect® probes, Agilent Technologies). Sequence capture using probes generally depends, in part, on the number of consecutive nucleotides in at least a portion of the target nucleic acid sequence that is complementary (or nearly complementary) to the sequence of the probe. In some examples, probes can correspond to driver mutations.

Processing: As used herein, the terms “processing”, “calculating”, and “comparing” can be used interchangeably. In certain applications, the terms refer to determining a difference, e.g., a difference in number or sequence. For example, gene expression, copy number variation (CNV), indel, and/or single nucleotide variant (SNV) values or sequences can be processed.

Processor. As used herein, “processor” refers to any circuit or virtual circuit (a physical circuit emulated by logic executing on an actual processor) that manipulates data values according to control signals (e.g., “commands,” “op codes,” “machine code,” etc.) and which produces corresponding output signals that are applied to operate a machine. A processor may, for example, be a CPU, a RISC processor, a CISC processor, a GPU, a DSP, an ASIC, a RFIC or any combination thereof. A processor may further be a multi-core processor having two or more independent processors (sometimes referred to as “cores”) that may execute instructions contemporaneously.

Promoter Region As used herein, “promoter region” refers to a DNA sequence recognized by the synthetic machinery of the cell, or introduced synthetic machinery, required to initiate the specific transcription of a gene.

Quantitative Measures: As used herein, “quantitative measures” refers to an absolute or relative measure. A quantitative measure can be, without limitation, a number, a statistical measurement (e.g., frequency, mean, median, standard deviation, or quantile), or a degree or a relative quantity (e.g., high, medium, and low). A quantitative measure can be a ratio of two quantitative measures. A quantitative measure can be a linear combination of quantitative measures. A quantitative measure may be a normalized measure.

Reference Sequence: As used herein, “reference sequence” refers to a known sequence used for purposes of comparison with experimentally determined sequences. For example, a known sequence can be an entire genome, a chromosome, or any segment thereof. A reference sequence can include at least about 20, at least about 50, at least about 100, at least about 200, at least about 250, at least about 300, at least about 350, at least about 400, at least about 450, at least about 500, at least about 1000, or more nucleotides. A reference sequence can align with a single contiguous sequence of a genome or chromosome or can include non-contiguous segments that align with different regions of a genome or chromosome. Example reference sequences, include, for example, human genome reference sequences, such as, hG19 and hG38.

Sample: As used herein, “sample” means anything capable of being analyzed by the methods and/or systems disclosed herein.

Sensitivity. As used herein, “sensitivity” means the probability of detecting the presence of a single nucleotide variant, an insertion, and a deletion at a given MAF and coverage and the probability of detecting the presence of a copy number variant at a given tumor fraction and coverage.

Sequencing: As used herein, “sequencing” refers to any of a number of technologies used to determine the sequence (e.g., the identity and order of monomer units) of a biomolecule, e.g., a nucleic acid such as DNA or RNA. Example sequencing methods include, but are not limited to, targeted sequencing, single molecule real-time sequencing, exon or exome sequencing, intron sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLID™ sequencing, MS-PET sequencing, and a combination thereof. In some implementations, sequencing can be performer by a gene analyzer such as, for example, gene analyzers commercially available from Illumina, Inc., Pacific Biosciences, Inc., or Applied Biosystems/Thermo Fisher Scientific, among many others.

Single Nucleotide Variant: As used herein, “single nucleotide variant” or “SNV” means a mutation or variation in a single nucleotide that occurs at a specific position in the genome.

Somatic Mutation: As used herein, “somatic mutation” means a mutation in the genome that occurs after conception. Somatic mutations can occur in any cell of the body except germ cells and accordingly, are not passed on to progeny.

Specifically binds: As used herein, “specifically binds” in the context of a probe or other oligonucleotide and a target sequence means that under appropriate hybridization conditions, the oligonucleotide or probe hybridizes to its target sequence, or replicates thereof, to form a stable probe: target hybrid, while at the same time formation of stable probe: non-target hybrids is minimized. Thus, a probe hybridizes to a target sequence or replicate thereof to a sufficiently greater extent than to a non-target sequence, to enable capture or detection of the target sequence. Appropriate hybridization conditions are well-known in the art, may be predicted based on sequence composition, or can be determined by using routine testing methods (see, e.g., Sambrook et al., Molecular Cloning, A Laboratory Manual, 2nd ed. (Cold Spring Harbor Laboratory Press, Cold Spring Harbor, NY, 1989) at §§ 1.90-1.91, 7.37-7.57, 9.47-9.51 and 11.47-11.57, particularly §§ 9.50-9.51, 11.12-11.13, 11.45-11.47 and 11.55-11.57, incorporated by reference herein).

Subject: As used herein, “subject” refers to an animal, such as a mammalian species (e.g., human) or avian (e.g., bird) species, or other organism, such as a plant. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual that is in need of therapy or suspected of needing therapy. The terms “individual” or “patient” are intended to be interchangeable with “subject.”

For example, a subject can be an individual who has been diagnosed with having a cancer, is going to receive a cancer therapy, and/or has received at least one cancer therapy. The subject can be in remission of a cancer. As another example, the subject can be an individual who is diagnosed of having an autoimmune disease. As another example, the subject can be a female individual who is pregnant or who is planning on getting pregnant, who may have been diagnosed of or suspected of having a disease, e.g., a cancer, an auto-immune disease.

Target Region: As used herein, “target region” refers to a genomic locus targeted for identification and/or capture, for example, by using probes (e.g., through sequence complementarity). A “target region set” or “set of target regions” refers to a plurality of genomic loci targeted for identification and/or capture, for example, by using a set of probes (e.g., through sequence complementarity).

Threshold: As used herein, “threshold” refers to a predetermined value used to characterize experimentally determined values of the same parameter for different samples depending on their relation to the threshold.

Tumor Fraction: As used herein, “tumor fraction” refers to the estimate of the fraction of nucleic acid molecules derived from a tumor in a given sample. For example, the tumor fraction of a sample can be a measure derived from the max MAF of the sample or pattern of sequencing coverage of the sample or length of the cfDNA fragments in the sample or any other selected feature of the sample. In some instances, the tumor fraction of a sample is equal to the max MAF of the sample.

Variant: As used herein, a “variant” can be referred to as an allele. A variant is usually presented at a frequency of 50% (0.5) or 100% (1), depending on whether the allele is heterozygous or homozygous. For example, germline variants are inherited and usually have a frequency of 0.5 or 1. Somatic variants; however, are acquired variants and usually have a frequency of <0.5. Major and minor alleles of a genetic locus refer to nucleic acids harboring the locus in which the locus is occupied by a nucleotide of a reference sequence, and a variant nucleotide different than the reference sequence respectively. Measurements at a locus can take the form of allelic fractions (AFs), which measure the frequency with which an allele is observed in a sample.

### DETAILED DESCRIPTION

Cancer is usually caused by the accumulation of mutations within genes of an individual's cells, at least some of which result in improperly regulated cell division. Such mutations can include single nucleotide variations (SNVs), gene fusions, insertions, transversions, translocations, and inversions. These mutations can also include copy number variations that correspond to an increase or a decrease in the number of copies of a gene within a tumor genome relative to an individual's noncancerous cells. An extent of mutations present in cell-free nucleic acids and an amount of mutated cell-free nucleic acids of a sample can be used as biomarkers to determine tumor progression, predict patient outcome, and refine treatment choices. In various examples, the extent of mutations present in cell-free nucleic acids can be indicated by tumor cells copy number and tumor fraction for a given sample.

Additionally, cancer can be indicated by non-sequence modifications, such as methylation. Examples of methylation changes in cancer include local gains of DNA methylation in the CpG islands at the TSS of genes involved in normal growth control, DNA repair, cell cycle regulation, and/or cell differentiation. This increased amount of methylation can be associated with an aberrant loss of transcriptional capacity of involved genes and occurs at least as frequently as point mutations and deletions as a cause of altered gene expression.

Thus, DNA methylation profiling can be used to detect aberrant methylation in DNA of a sample. The DNA can correspond to certain genomic regions (“differentially methylated regions” or “DMRs”) that are normally hypermethylated or hypomethylated in a given sample type (e.g., cfDNA from the bloodstream) but which may show an abnormal degree of methylation that correlates to a neoplasm or cancer, e.g., because of unusually increased contributions of tissues to the type of sample (e.g., due to increased shedding of DNA in or around the neoplasm or cancer) and/or from extents of methylation of the genome that are altered during development or that are perturbed by disease, for example, cancer or any cancer-associated disease.

Some methods of measuring DNA methylation can make accurately determining an amount of methylation of DNA difficult. The accuracy with which DNA methylation is determined can impact the accuracy of estimates of tumor fraction for samples. Since tumor fraction can be used to determine whether a sample is derived from a subject in which a tumor is present or not, the accuracy of determination of tumor fraction estimates can impact diagnosis and/or treatment decisions for individuals.

The methods and systems described herein are directed to analyzing single site methylation data obtained from samples that include at least some cell-free DNA (cfDNA) samples. In at least some implementations, one or more paired datasets can be analyzed to determine methylation features of cfDNA molecules that are indicative of a tumor being present in a subject. In one or more examples, a first paired dataset can comprise a first dataset derived from one or more plasma samples obtained from a test subject and a second dataset derived from samples obtained from individuals in which a tumor is not detected. In various examples, the second dataset can comprise a background dataset. Sequence representations generated from the first dataset and from the second dataset can be analyzed to determine overlapping CpG regions of nucleic acid molecules derived from the test subject plasma sample(s) and the background dataset. Additionally, methylation data for the overlapping CpG regions can be analyzed to determine information that can indicate whether a tumor is present in the test subject. The methylation data can be analyzed based on one or more experimentally determined criteria to generate the tumor information.

In one or more additional examples, a second paired dataset can comprise the first dataset derived from the one or more plasma samples obtained from the test subject and a third dataset that corresponds to molecules derived from one or more tissue samples. In one or more illustrative examples, the one or more tissue samples can be obtained from one or more individuals in which a tumor is detected. In at least some examples, the third dataset can comprise a tumor tissue dataset. Additional sequence representations generated from the first dataset and from the third dataset can be analyzed to determine additional overlapping CpG regions of nucleic acid molecules derived from the test subject plasma sample(s) and the tumor tissue dataset. Further, methylation data for the additional overlapping CpG regions can be analyzed to determine information that can indicate whether a tumor is present in the test subject. The methylation data can be analyzed based on one or more experimentally determined criteria to generate the tumor information. In various examples, information derived from an analysis of the first paired dataset and the second paired dataset can be combined to determine an indication of a tumor being present in the test subject.

In at least some existing systems that analyze methylation data to determine tumor information use an analysis of specific methylation data, such as methylation rate and/or haplotype structure. In these scenarios, relying on a particular type of methylation data to determine tumor information can lead to inaccurate tumor information. Instead, the methods, processes, systems, techniques, and frameworks described herein implement an analysis of one or more paired datasets that are agnostic with respect to specific methylation data and implement various criteria and computational analyses to determine tumor information. In contrast to existing techniques, by not relying on specific methylation data, the implementations herein can determine tumor information that is more accurate and more reliable for determining cancer diagnoses and for determining cancer-related treatments.

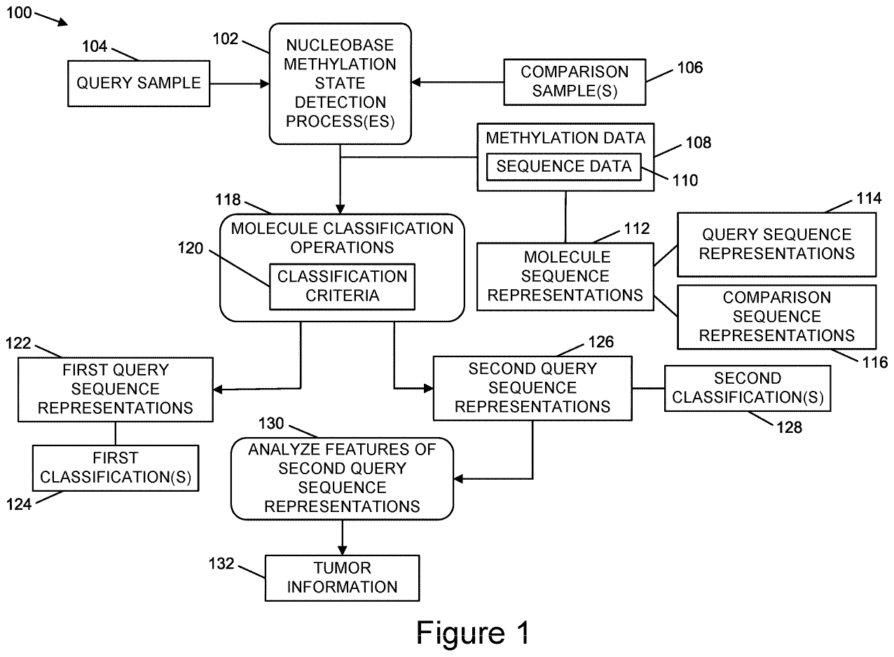

FIG. 1 is a diagrammatic representation of an example architecture 100 that analyzes methylation states of nucleic acid molecules derived from samples obtained from different groups of subjects, according to one or more example implementations. In one or more examples, the architecture 100 can analyze information from one or more paired datasets to determine biological condition, wherein the biological condition comprises disease information. The disease information can indicate that one or more diseases are present within one or more subjects. The disease information can be related to damaged organ tissue or organ failure. For example, the disease information can indicate damage to at least one of lung tissue, heart tissue, brain tissue, liver tissue, kidney tissue, intestinal tissue, stomach tissue, pancreas tissue, or skin tissue. In various examples, the disease information can indicate organ failure in one or more subject. In one or more additional examples, the disease information can indicate one or more neurological disorders, Alzheimer's disease, autoimmune diseases, autoinflammatory diseases, multiple sclerosis, Parkinson's disease or cystic fibrosis.

In various examples, the architecture 100 can analyze information from one or more paired datasets to determine tumor information. The tumor information can correspond to one or more diseases. In one or more examples, the disease under consideration is a type of cancer. Non-limiting examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic (CLL), chronic myeloid (CML), chronic myelomonocytic (CMML), liver cancer, liver carcinoma, hepatoma, hepatocellular carcinoma, cholangiocarcinoma, hepatoblastoma, lung cancer, non-small cell lung cancer (NSCLC), mesothelioma, B-cell lymphomas, non-Hodgkin lymphoma, diffuse large B-cell lymphoma, Mantle cell lymphoma, T-cell lymphomas, non-Hodgkin lymphoma, precursor T-lymphoblastic lymphoma/leukemia, peripheral T-cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas, prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma.

The architecture 100 includes one or more nucleobase methylation state detection processes 102 for a query sample 104 and for one or more comparison samples 106. The query sample 104 can include a biological fluid sample obtained from a test subject. In one or more examples, the query sample 104 can include a blood-based sample obtained from the test subject. In one or more additional examples, the query sample 104 can include a plasma sample obtained from the test subject. The query sample 104 can also include a tissue sample obtained from the test subject. Additionally, the query sample 104 can include a cellular sample obtained from the test subject. To illustrate, the query sample 104 can include a peripheral blood mononuclear cells (PBMCs). In still other examples, the query sample 104 can include a whole blood sample obtained from the test subject.

The one or more comparison samples 106 can be derived from one or more additional subjects. The one or more comparison samples 106 can include one or more biological fluid samples obtained from the one or more additional subjects. In one or more examples, the one or more comparison samples 106 can include one or more blood-based samples obtained from the one or more additional subjects. In one or more additional examples, the one or more comparison samples 106 can include one or more plasma samples obtained from the one or more additional subjects. In addition, the one or more comparison samples 106 can include one or more tissue samples obtained from the one or more additional subjects. In various examples, at least a portion of the one or more tissue samples can be obtained from the test subject. In one or more further examples, the one or more comparison samples 106 can include buffy coat obtained from the test subject. In still other examples, the one or more comparison samples 106 can include buffy coat obtained from one or more additional subjects. In at least some examples, a tumor may not be detected in the one or more additional subjects.

The one or more subjects providing at least one of the query sample 104 or the one or more comparison samples 106 can include one or more mammals. In one or more additional examples, the one or more subjects providing at least one of the query sample 104 or the one or more comparison samples 106 can include one or more humans. In one or more further illustrative examples, the one or more subjects providing at least one of the query sample 104 or the one or more comparison samples 106 can include one or more non-human mammals.

The one or more nucleobase methylation state detection processes 102 can include one or more chemical processes and/or biochemical processes that impact a first type of nucleotide differently than a second type of nucleotide. For example, the one or more nucleobase methylation state detection processes 102 can include one or more reactions that cause at least one atomic and/or molecular moiety of the first type of nucleotide to be modified in a manner that is different from the manner in which the one or more reactions affect the second type of nucleotide. In one or more examples, the impact of the one or more nucleobase methylation state detection processes 102 on a given type of nucleotide can be based on one or more previous modifications to the given type of nucleotide in relation to an unmodified form of the given type of nucleotide. That is, in various examples, a molecule corresponding to a given type of nucleotide may have been modified before being subjected to the one or more nucleobase methylation state detection processes 102. To illustrate, before being subjected to the one or more nucleobase methylation state detection processes 102, nucleotides of nucleic acid molecules derived from at least one of the query sample 104 or the one or more comparison samples 106 can be modified due to mutations caused by the presence of a tumor in a subject. In at least some examples, the one or more nucleobase methylation state detection processes 102 can modify the first type of nucleotide or the second type of nucleotide such that the nucleobase pairing of the first type of nucleotide or the second type of nucleotide is altered.

In one or more illustrative examples, the one or more nucleobase methylation state detection processes 102 can be performed on nucleic acid molecules included in the query sample 104 and the one or more comparison samples 106. The one or more nucleobase methylation state detection processes 102 can modify a first type of nucleotide of the nucleic acid molecules in a first manner and one or more additional types of nucleotides of the nucleic acid molecules in a second manner. To illustrate, the one or more nucleobase methylation state detection processes 102 can modify at least one of cytosines, guanines, thiamines, or adenines differently than at least one other of cytosines, guanines, thiamines, or adenines. In at least some examples, the one or more nucleobase methylation state detection processes 102 can modify cytosines differently than guanines, thiamines, or adenines. In various examples, the one or more nucleobase methylation state detection processes 102 can modify cytosines such that the modified cytosines no longer pair with guanines. For example, the one or more nucleobase methylation state detection processes 102 can convert cytosines of the nucleic acid molecules included in the query sample 104 and the one or more comparison samples 106 to uracils. In still other examples, the one or more nucleobase methylation state detection processes 102 may not modify cytosines that were methylated prior to being subjected to the one or more nucleobase methylation state detection processes 102. In one or more examples, the one or more nucleobase methylation state detection processes 102 may not modify 5-methylcytosines and/or 5-hydroxymethylcytosines of nucleic acid molecules derived from the query sample 104 and the one or more comparison samples 106. In this way, the one or more nucleobase methylation state detection processes 102 can be used to differentiate cytosines that have been previously modified to include a 5-methyl group versus previously unmodified cytosines.

In one or more examples, the one or more nucleobase methylation state detection processes 102 can include at least one of sodium bisulfite conversion and sequencing, Tet-assisted bisulfite sequencing (TAB-Seq), differential enzymatic cleavage, one or more single molecule sequencing methods, such as nanopore DNA sequencing, oxidative bisulfite (Ox-BS) conversion, APOBEC-coupled epigenetic (ACE) conversion, Enzymatic Methyl Sequencing (EM-Seq), single-enzyme 5-methylcytosine sequencing (SEM-seq), or direct methylation sequencing (DM-Seq).

In one or more additional examples, the one or more nucleobase methylation state detection processes 102 can include one or more processes that separate nucleic acid molecules based on amounts of nucleotides of the nucleic acid molecules that have been previously modified. For example, the one or more nucleobase methylation state detection processes 102 can determine a methylation rate for one or more regions of the nucleic acid molecules derived from the query sample 104 and the one or more comparison samples 106. In various examples, the one or more nucleobase methylation state detection processes 102 can separate nucleic acid molecules included in the query sample 104 and the one or more comparison samples 106 based on amounts of methylated cytosines included in CG regions of individual nucleic acid molecules. To illustrate, the one or more nucleobase methylation state detection processes 102 can separate the nucleic acid molecules derived from the query sample 104 and the one or more comparison samples 106 into a plurality of groups of nucleic acid molecules with individual groups of nucleic acid molecules corresponding to respective amounts of methylated cytosines of the nucleic acid molecules 104. The one or more nucleobase methylation state detection processes 102 can include at least one of partitioning of nucleic acid molecules included in the query sample 104 and included in the one or more comparison samples 106 based on a strength of binding of the individual nucleic acid molecules to methyl binding domain (MBD) and, optionally, treatment with methylation sensitive restriction enzyme (MSRE) and/or methylation dependent restriction enzyme (MDRE). In various examples, a strength of binding of nucleic acid molecules to MBD can be determined by subjecting the nucleic acids to a series of washes having different concentrations of MBD.

The one or more nucleobase methylation state detection processes 102 can generate methylation data 108. The methylation data 108 can indicate positions of nucleic acid molecules derived from the query sample 104 and the one or more comparison samples 106 that include a methylated cytosine. That is, in various examples, the methylation data 108 can indicate positions of nucleic acid molecules derived from the query sample 104 and the one or more comparison samples 106 where at least one of a 5-methylcytosine and/or a 5-hydroxymethylcytosine is located. For example, the methylation data 108 can indicate discrete, individual positions of individual nucleic acid molecules derived from the query sample 104 and the one or more comparison samples 106 that include at least one of a 5-methylcytosine and/or a 5-hydroxymethylcytosine. In one or more additional examples, the methylation data 108 can indicate a group of positions of individual nucleic acid molecules derived from the query sample 104 and the one or more comparison samples 106 that include at least one of a 5-methylcytosine and/or a 5-hydroxymethylcytosine.

In at least some examples, the nucleobase methylation state detection processes 102 can include one or more sequencing processes. For example, the nucleobase methylation state detection processes 102 can include whole genome bisulfite sequencing, reduced representation bisulfite sequencing, targeted bisulfite sequencing, extended-representation bisulfite sequencing, or one or more combinations thereof. In one or more illustrative examples, whole genomic bisulfite sequencing can be performed according to the techniques described in T. Gong et al., “Analysis and performance assessment of the whole genome bisulfite sequencing data workflow: currently available tools and a practical guide to advance DNA methylation studies,” Small Methods, 6: e2101251, 2022. In one or more additional illustrative examples, reduced representation bisulfite sequencing can be performed according to techniques described in Meissner, A., Gnirke, A., Bell, G. W., Ramsahoye, B., Lander, E. S., and Jaenisch, R. (2005). Reduced representation bisulfite sequencing for comparative high-resolution DNA methylation analysis. Nucleic acids research 33, 5868-5877. In one or more further illustrative examples, targeted bisulfite sequencing can be performed according to techniques described in D. A. Moser et al., “Targeted bisulfite sequencing: A novel tool for the assessment of DNA methylation with high sensitivity and increased coverage,” Psychoneuroendocrinology, 120:1-8, 2020 and/or E. Leitão et al., “Locus-specific DNA methylation analysis by targeted deep bisulfite sequencing,” Methods Mol Biol, 1767:351-66, 2018. In still further illustrative examples, extended-representation bisulfite sequencing can be performed according to techniques described in Shareef, S. J., Bevill, S. M., Raman, A. T. et al. Extended-representation bisulfite sequencing of gene regulatory elements in multiplexed samples and single cells. Nat Biotechnol 39, 1086-1094 (2021).

In at least some implementations, the methylation data 108 can include sequence data 110. The sequence data 110 can include alphanumeric representations of the nucleic acid molecules derived from the query sample 104 and the one or more comparison samples 106. For example, the sequence data 110 can include, for individual nucleic acids, data that corresponds to a string of letters that represents the respective chains of nucleotides that correspond to the individual nucleic acid molecules derived from the query sample 104 and the one or more comparison samples 106. At least one of the methylation data 108 or the sequence data 110 can be stored in one or more data files. For example, the sequence data 110 can be stored in a FASTQ file that comprises a text-based sequencing data file format storing raw sequence data and quality scores. In one or more additional examples, the sequence data 110 can be stored in a data file according to a binary base call (BCL) sequence file format. In one or more further examples, the sequence data 110 can be stored in a BAM file. In one or more examples, the sequence data 110 can comprise at least about one gigabyte (GB), at least about 2 GB, at least about 3 GB, at least about 4 GB, at least about 5 GB, at least about 8 GB, or at least about 10 GB. An individual sequence representation included in the sequence data 110 can be referred to herein as a “read” or a “sequencing read.” In various examples, individual nucleic acid molecules derived from the query sample 104 and the one or more comparison samples 106 can correspond to multiple sequence representations included in the sequence data 110 as a result of the amplification of the individual nucleic acid molecules that takes place as part of the one or more nucleobase methylation state detection processes 102. In situations where amplification of nucleic acids is not performed as part of the one or more nucleobase methylation state detection processes 102, individual nucleic acid molecules derived from the query sample 104 and the one or more comparison samples 106 can correspond to a single sequence representation included in the sequence data 142 as a result of the absence of amplification of the individual nucleic acid molecules.

When multiple sequence representations are present in the sequence data 110 that correspond to a single nucleic acid molecule derived from the query sample 104 and the one or more comparison samples 106, a number of groups can be generated from the sequence representations with each group corresponding to a single nucleic acid molecule derived from the query sample 104 and/or the one or more comparison samples 106. In various examples, the groups of sequence representations included in the sequence data 110 that correspond to a single nucleic acid molecule can be referred to herein as “families.” In at least some examples, start and stop positions with respect to a reference sequence having a common molecular barcode can be used to determine groups of the sequence representations that correspond to individual nucleic acid molecules. In one or more illustrative examples, an individual sequence representation that represents a family of sequence representations and that corresponds to a single nucleic acid molecule derived from the query sample 104 and/or the one or more comparison samples 106 can be referred to herein as a “consensus sequence representation.”

In one or more examples, consensus sequence representations generated from the sequence data 110 can be used to generate molecule sequence representations 112. In various examples, individual molecule sequence representations 112 can correspond to individual nucleic acid molecules derived from the query sample 104 and the one or more comparison samples 106. In at least some examples, the methylation data 108 can indicate, for at least a portion of the individual positions of the individual molecule sequence representations 112, a methylation state of a nucleotide present at an individual position. In one or more illustrative examples, the methylation data 108 can indicate one or more individual positions of the individual molecule sequence representations 112 having methylated cytosines. The molecule sequence representations 112 can include query sequence representations 114. The query sequence representations 114 can correspond to individual nucleic acid molecules derived from the query sample 104. Additionally, the molecule sequence representations 112 can include comparison sequence representations 116. The comparison sequence representations 116 can correspond to individual nucleic acid molecules derived from the one or more comparison samples 106.

The architecture 100 can also include molecule classification operations 118. The molecule classification operations 118 can include analyzing the methylation data 108 to determine a group of molecules derived from the query sample 104 having methylation features that can be indicative of a tumor being present in a test subject that provided the query sample 104. In one or more examples, the molecule classification operations 118 can analyze methylation features of positions of the query sequence representations 114 in relation to methylation features of positions of the comparison sequence representations 116 to determine a group of molecules derived from the query sample 104 having methylation features that can be indicative of a tumor being present in a test subject that provided the query sample 104.

The molecule classification operations 118 can implement one or more classification criteria 120. The molecule classification operations 118 can be implemented using the one or more classification criteria 120 with respect to the molecule sequence representations 112 to determine a portion of the query sequence representations 114 that correspond to nucleic acid molecules derived from the query sample 104 that can have methylation features indicating the presence of a tumor in the test subject. In one or more illustrative examples, the classification criteria 120 can include at least one of one or more rules, one or more schemes, or one or more frameworks that can be implemented to determine a portion of the query sequence representations 114 that correspond to nucleic acid molecules derived from the query sample 104 having methylation features that can be indicative of a tumor being present in the test subject. In at least some examples, at least one of one or more rules, one or more schemes, or one or more frameworks included in the classification criteria 120 can be determined using an analysis of one or more experimental datasets that determines one or more values, one or more ranges, one or more functions, or one or more combinations thereof, that can be used to identify query sequence representations 114 that correspond to nucleic acid molecules derived from the query sample 104 having methylation features indicative of a tumor being present in the test subject.

In one or more additional illustrative examples, the classification criteria 120 can be part of one or more computational models that can be used to analyze the molecule sequence representations 112 to determine a portion of the query sequence representations 114 that correspond to nucleic acid molecules derived from the query sample 104 having methylation features indicative of a tumor being present in the test subject. In various examples when the classification criteria 120 are part of one or more computational models, the one or more classification criteria 120 can be determined using a training process for the one or more computational models. The methylation features of nucleic acid molecules that can be indicative of a tumor being present in a subject can correspond to one or more variables and/or one or more nodes included in the one or more computational models. The one or more variables and/or one or more nodes included in the one or more computational models in addition to one or more weights associated with the one or more variables and/or one or more nodes can be determined through an analysis of a corpus of data that can be stored by one or more data stores coupled to or otherwise in electronic communication with one or more computing systems that implement one or more portions of the architecture 100. The corpus of data can include training data corresponding to nucleic acid molecules derived from training samples of individuals in which a tumor is detected and, optionally, corresponding to nucleic acid molecules derived from training samples of individuals in which a tumor is not detected. Additionally, the corpus of data can include information obtained from data sources that are external to one or more computing systems implementing one or more portions of the architecture 100, such as one or more publicly accessible data sources and/or one or more privately accessible data sources. In at least some examples, the corpus of data used to train the one or more computational models can include information related to the treatment of individuals previously diagnosed with cancer. In one or more illustrative examples, at least a portion of the corpus of data can be obtained by an external data extraction and storage system that ingests data from one or more data files, one or more websites, one or more databases, or one or more combinations thereof.

In one or more examples, before being used to generate one or more computational models that implement the molecule classification operations 118, training data can be preprocessed. The data preprocessing can include one or more operations to modify at least a portion of the corpus of data or other training data such that information included in the corpus of data or other training data can be analyzed to generate the one or more computational models. The data preprocessing can include formatting at least a portion of the corpus of data according to one or more formatting schemes. In various examples, the data preprocessing can standardize the format of the information included in at least a portion of the corpus of data. Additionally, the data preprocessing can include at least one of aggregating one or more portions of the corpus of data or separating one or more portions of the corpus of data.

After performing the data preprocessing, at least a portion of the corpus of data can be used as training data to generate the one or more computational models. In one or more implementations, the training data can be analyzed by performing one or more feature extraction operations and one or more classification operations. Feature extraction operations can include identifying one or more variables and/or one or more sets of variables that can be used to make one or more predictions based on a set of input data. The feature extraction can determine relationships between one or more variables included in the training data and determine one or more measures of correlations between variables and/or groups of variables included in the training data. The classification operations can include classifying one or more pieces of information included in the training data according to one or more categories.

The feature extraction and classification operations can be used to determine one or more initial computational models that can make one or more determinations and/or predictions directed to tumor information associated with the training data. In various examples, the feature extraction and classification operations can identify one or more methylation features of cell-free DNA molecules that can be indicative of a tumor being present in an individual. Additionally, evaluation data can be used to evaluate the performance of the initial computational models produced by the feature extraction at operations and the classification operations. The evaluation data can include a portion of the corpus of data that is different from the training data. In this way, the evaluation data that is used to evaluate the performance of the initial computational models is different from the training data that is used to generate the one or more initial computational models. Based on the performance of the initial computational models implemented with the evaluation data, the feature extraction and/or classification operations can be performed one or more additional times until the one or more computational models are produced. The one or more computational models can be produced after one or more iterations of feature extraction, classification, and prediction using the evaluation data. In one or more implementations, the one or more computational models can be produced after the feature extraction, classification, and prediction operations produce computational models that satisfy one or more performance criteria, such as one or more convergence criteria or one or more accuracy criteria.

After the one or more computational models have been generated, the one or more computational models can be further refined as additional data is obtained. For example, as additional methylation data and sequence data are obtained, the accuracy of the one or more computational models can be further evaluated. In various examples, as the new methylation data and sequence data is obtained, model output/feedback can be produced that is then fed back to the classification operations. In this way, the new data can be used to improve the predictions made by the one or more computational models by modifying one or more variables, one or more nodes, and/or one or more weights of the one or more computational models.

The one or more computational models can implement one or more machine learning techniques. For example, the one or more computational models can include one or more artificial neural networks. To illustrate, the one or more computational models can include at least one of one or more convolutional neural networks, one or more recurrent neural networks, or one or more multilayer perceptron neural networks. Additionally, at least a portion of the one or more computational models can be implemented by executing one or more machine learning techniques at one or more local computing device and/or one or more remotely located computing devices, such as a cloud-based computational platform.

In various examples, the molecule classification operations 118 can include determining a number of the query sequence representations 114 that are aligned with a number of the comparison sequence representations 116. A query sequence representation 114 and a comparison sequence representation 116 can be aligned when nucleotides at a threshold number of positions of the query sequence representation 114 and the comparison sequence representation 116 correspond to a same genomic region of a reference sequence. In this way, the molecule classification operations 118 can generate a group of aligned sequence representations that include a coupling or other association between at least one query sequence representation 114 and at least one comparison sequence representations 116.

The molecule classification operations 118 can include analyzing the group of aligned sequence representations based on the classification criteria 120. In one or more examples, the classification criteria 120 can indicate, for a given pairing between a query sequence representation 114 and a comparison sequence representation 116, a number of common locations for CpG dinucleotides of the query sequence representation 114 and the comparison sequence representation 116. In one or more additional examples, the classification criteria 120 can indicate an amount of correlation between methylation states of aligned sequence representations. In one or more further examples, the classification criteria 120 can indicate an order in which to apply a number of classification criteria 120. In one or more illustrative examples, the classification criteria 120 can indicate a first molecule classification operation that includes analyzing the molecule sequence representations 112 to determine a number of aligned query sequence representations 114 and comparison sequence representations having at least a threshold number of common CpG dinucleotide position to generate a first group of sequence representations. The classification criteria 120 can also indicate that a second molecule classification operation is to be performed after the first molecule classification operation to determine a second group of aligned sequence representations having a same methylation state in at least a threshold number of common CpG regions.

The molecule classification operations 118 can implement the classification criteria 120 to determine classifications for nucleic acid molecules derived from the query sample 104. In one or more examples, the molecule classification operations 118 can implement the classification criteria 120 to determine first query sequence representations 122 having one or more first classifications 124. In at least some examples, the molecule classification operations 118 can determine that the first query sequence representations 122 satisfy one or more classification criteria 120 with respect to at least a portion of the comparison sequence representations 116. In one or more illustrative examples, the first query sequence representations 122 can satisfy one or more sequence classification criteria and/or one or more methylation classification criteria with respect to at least a portion of the comparison sequence representations 116. For example, individual first query sequence representations 122 can have nucleotide sequences that have at least a threshold amount of homology with one or more comparison sequence representations 116. Additionally, individual first query sequence representations 122 can have CpG regions with methylation states that correspond to methylation states of at least at threshold number of corresponding CpG regions of one or more comparison sequence representations 116. In various examples, the first query sequence representations 122 can correspond to a portion of the comparison sequence representations 116 that are derived from samples obtained from subjects in which a tumor is not detected.

Further, the molecule classification operations 118 can implement the classification criteria 120 to determine second query sequence representations 126 that have one or more second classifications 128. In at least some examples, the molecule classification operations 118 can determine that the second query sequence representations 126 do not satisfy one or more classification criteria 120 with respect to at least a portion of the comparison sequence representations 116. For example, the second query sequence representations 126 may not satisfy one or more sequence classification criteria and/or one or more methylation classification criteria with respect to at least a portion of the comparison sequence representations 116. To illustrate, second query sequence representations 126 can have nucleotide sequences that less than a threshold amount of homology with the comparison sequence representations 116. Additionally, individual first query sequence representations 122 can have CpG regions with methylation states that correspond to methylation states of less than a threshold number of corresponding CpG regions of the comparison sequence representations 116. In various examples, the second query sequence representations 126 can have sequence features and/or methylation features that are different from a portion of the comparison sequence representations 116 that correspond to nucleic acid molecules derived from the one or more comparison samples 106 obtained from subjects in which a tumor is not detected.

In still other examples, a portion of the comparison sequence representations 116 can correspond to nucleic acid molecules derived from at least a portion of the one or more comparison samples 106 obtained from one or more subjects in which a tumor is detected. In these scenarios, the molecule classification operations 118 can apply the classification criteria 120 to determine at least a portion of the second query sequence representations 126 that satisfy at least one of sequence criteria or methylation criteria in relation to one or more comparison sequence representations 116 derived from one or more comparison samples 106 derived from subjects in which a tumor is detected. In one or more examples, the molecule classification operations 118 can determine second query sequence representations 126 that have at least a threshold number of CpG regions as one or more comparison sequence representations 116 derived from one or more comparison samples 106 obtained from subject in which a tumor is present and that correspond to a same genomic region of a reference sequence. Additionally, the molecule classification operations 118 can determine second query sequence representations 126 that have CpG regions with methylation states that correspond to methylation states of at least a threshold number of corresponding CpG regions of the comparison sequence representations 116 that are derived from one or more comparison samples 106 obtained from one or more subject in which a tumor is detected. In one or more illustrative examples, the one or more comparison samples 106 obtained from one or more subjects in which a tumor is present can include one or more tissue samples. In one or more additional illustrative examples, the one or more comparison samples 106 obtained from one or more subjects in which a tumor is present can include one or more tissue samples obtained from the test subject.

The architecture 100 can include, at operation 130, analyzing features of the second query sequence representations 126 to determine tumor information 132. In one or more examples, operation 130 can include analyzing an amount of the second query sequence representations 126 in relation to an amount of the molecule sequence representations 112. In one or more additional examples, operation 130 can include analyzing a number of the second query sequence representations 126 with respect to a total number of the molecule sequence representations 112. In one or more further examples, operation 130 can include determining at least a portion of the second query sequence representations 126 that correspond to one or more specified genomic regions. For example, for individual genomic regions of a reference sequence, a number of the second query sequence representations 126 that have at least a threshold amount of overlap with the individual genomic regions can be determined. In one or more illustrative examples, the specified genomic regions can include one or more classification regions. The one or more classification regions can include genomic regions of a reference sequence that are enriched as part of a panel used to determine at least one of the presence of cancer in subjects, a stage of cancer in subjects, an amount of progression of cancer in subjects, or one or more combinations thereof. In one or more additional illustrative examples, the classification regions can include one or more differentially methylated regions. In various examples, the operation 130 can include analyzing a number of the second query sequence representations 126 that correspond to one or more classification regions with respect to an amount of the molecule sequence representations 112, such as a total number of the molecule sequence representations 112.

The tumor information 132 can include an indication of whether or not a tumor is present in the test subject. In addition, the tumor information 132 can indicate a probability of a tumor being present in the test subject. The tumor information 132 can also indicate a stage of cancer present in the test subject. Further, the tumor information 132 can indicate a progression of cancer in the test subject. In still other examples, the tumor information 132 can indicate one or more types of cancer present in the test subject. In various examples, the tumor information 132 can be used to determine one or more treatments to provide to the test subject in situations where the tumor information 132 indicates the presence of a tumor in the test subject and/or a progression of cancer in the test subject. In one or more examples, the tumor information 132 can correspond to a tumor fraction for the test subject.

In one or more additional examples, the tumor information 132 can be determined for a test subject at multiple times over a period of time. To illustrate, the tumor information 132 can be obtained by collecting samples from the test subject at different points in time. In this way, longitudinal information can be generated for a test subject. In one or more further examples, the tumor information 132 can be determined at multiple times while the test subject is receiving treatment for one or more types of cancer. In this way, the tumor information 132 can be used to determine at least one of progression or regression of one or more types of cancer for a test subject.

In at least some examples, the tumor information 132 can also be determined by analyzing one or more additional data sources. For example, electronic health records of a test subject can be analyzed to determine the tumor information 132 for a test subject. In one or more examples, the electronic health records can include clinical observations recorded by a healthcare provider, laboratory test results, diagnostic test information, imaging information, dental health information, one or more combinations thereof, and the like. Additionally, the electronic health records of a test subject can include billing records that indicate payment information with respect to at least one of products or services provided to individuals by healthcare providers. Further, the electronic health records can include health insurance claims information that indicates information obtained by health insurance companies related to the treatment of individuals with respect to one or more biological conditions. In one or more examples, one or more computational algorithms, one or more machine learning models, and/or one or more statistical models can be applied to the electronic health records to determine one or more inferences related to the health of a test subject. In one or more illustrative examples, the electronic health records of a test subject can be analyzed using at least one of one or more computational algorithms, one or more machine learning models, or one or more statistical models to determine and/or to supplement the tumor information 132 for the test subject.

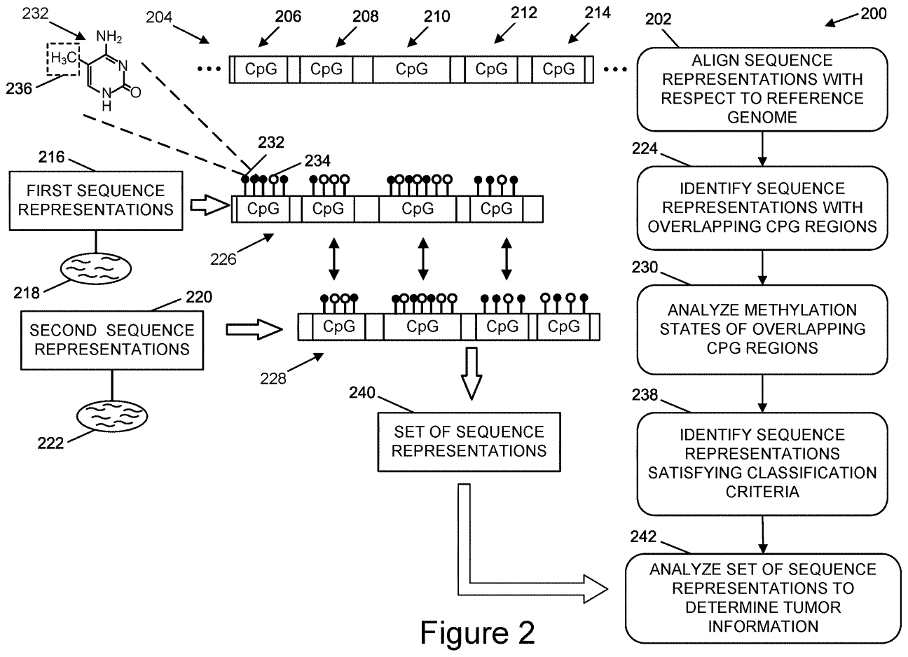

FIG. 2 is a diagrammatic representation of an example environment 200 to identify nucleic acid molecules obtained from different subjects that have overlapping cytosine-guanine regions to determine tumor information for one or more test subjects, according to one or more example implementations. The environment 200 can include, at operation 202, aligning sequence representations with respect to a reference sequence 204. The reference sequence 204 can include a number of regions that have at least a threshold number of cytosine molecules and guanine molecules. In one or more examples, the reference sequence 204 can include regions having at least a threshold number of cytosine-guanine dinucleotides. In various examples, at least a portion of the cytosine-guanine pairs included in the regions can be sequentially located in portions of the reference sequence 204. In one or more illustrative examples, a region of the reference sequence 204 or other nucleic acid molecule having at least a threshold amount of cytosine-guanine pairs can be referred to herein as a “CG region” or a “CpG region.” In the illustrative example of FIG. 2, the portion of the reference sequence 204 includes a first CpG region 206, a second CpG region 208, a third CpG region 210, a fourth CpG region 212, and a fifth CpG region 214.

In one or more examples, a CpG region can include at least 20 CpG dinucleotides, at least 50 CpG dinucleotides, at least 100 CpG dinucleotides, or at least 200 CpG dinucleotides. In one or more illustrative examples, a CG region can include from 200 CpG dinucleotides to 5000 CpG dinucleotides, from 300 CpG dinucleotides to 3000 CpG dinucleotides, from 200 CpG dinucleotides to 2500 CpG dinucleotides, or from 500 CpG dinucleotides to 1500 CpG dinucleotides. Additionally, a CG region can have a GC percentage of at least 50% and an observed-to-expected CpG ratio of at least 60%. The observed-to-expected CpG ratio can be calculated where the observed CpG is the number of CpGs identified in a given genomic region and the expected CpGs is the number of cytosines multiplied by the number of guanines divided by the number of bases in the genomic region. The expected CpGs can also be calculated by:

((number of cytosines+number of guanines)/2)²/length of genomic region.

For example, a CG region can be determined using the techniques described by Gardiner-Garden M, Frommer M (1987). “CpG islands in vertebrate genomes”. Journal of Molecular Biology. 196 (2): 261-282. and/or Saxonov S, Berg P, Brutlag D L (2006). “A genome-wide analysis of CpG dinucleotides in the human genome distinguishes two distinct classes of promoters”. Proc Natl Acad Sci USA. 103 (5): 1412-1417.

First sequence representations 216 derived from first sample molecules 218 can be aligned with the reference sequence 204. In one or more examples, the first sample molecules 218 can be derived from one or more samples obtained from a test subject. In addition, second sequence representations 220 derived from second sample molecules 222 can be aligned with the reference sequence 204. The second sample molecules 222 can be derived from one or more samples obtained from at least one of the test subject or one or more additional subjects. In various examples, at least a portion of the second sample molecules 222 can be derived from samples obtained from one or more individuals in which cancer is not detected. Further, at least a portion of the second sample molecules 222 can be derived from additional samples obtained from one or more additional individuals in which cancer is detected.

In one or more illustrative examples, the alignment process performed with respect to operation 202 can determine an amount of homology between individual first sequence representations 216 and portions of the reference sequence 204 and an amount of homology between individual second sequence representations 220 and portions of the reference sequence 204. In this way, the alignment process can also determine an amount of homology between individual first sequence representations 216 and individual second sequence representations 220. The amount of homology between a given sequence representation and the reference sequence 204 can indicate a number of positions of the reference sequence 204 that have the same nucleotide as corresponding positions of a given first sequence representation 216 and/or of a given second sequence representation 220. At least one of a first sequence representation 216 or a second sequence representation 220 can be aligned with a portion of the reference sequence 204 based on determining that at least one of a first sequence representation 216 or a second sequence representation 220 and the portion of the reference sequence 204 have at least a threshold amount of homology. In scenarios where a first sequence representation 216 or a second sequence representation 220 has at least the threshold amount of homology with respect to multiple portions of the reference sequence 204, the portion of the reference sequence 204 having the greatest amount of homology with the first sequence representation 216 or the second sequence representation 220 can be determined to be aligned with the first sequence representation 216 or the second sequence representation 220.

The amount of homology between a given sequence representation and a portion of the reference sequence 204 can be determined using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Genetics Computer Group, University Research Park, Madison Wis.), using default settings, which uses the algorithm of Needleman and Wunsch (J. Mol. Biol. 48; 443-453 (1970)). The amount of homology between a sequence representation and a portion of the reference sequence can also be determined using a Burrows-Wheeler aligner (Li, H., & Durbin, R. (2009). Fast and accurate short read alignment with Burrows-Wheeler transform. Bioinformatics, 25(14), 1754-1760).

The environment 200 can also include, at operation 224, identifying sequence representations with overlapping CpG regions. Overlapping sequence representations can include a first sequence representation 216 having at least one CpG region having positions of the reference sequence 204 that corresponds to an additional CpG region of a second sequence representation 220 that also includes the same positions of the reference sequence 204. In the illustrative example of FIG. 2, a first sequence representation 226 can have CpG regions that overlap with CpG regions of a second sequence representation 228. For example, the first sequence representation 226 and the second sequence representation 228 can overlap in the second CpG region 208, the third CpG region 210, and the fourth CpG region 212.

The environment 200 can include, at operation 230, analyzing methylation states of overlapping CpG regions. In one or more illustrative examples, the methylation states can correspond to unmethylated cytosines and methylated cytosines. An example methylated cytosine 232 is indicated at a position of the first CpG region 206 and an example unmethylated cytosine 234 is indicated at an additional position of the first CpG region 206. The methylated cytosine 232 includes a methyl group 236 at the 5-carbon position of a cytosine molecule. In the illustrative example of FIG. 2, the methylated cytosine includes 5-methylcytosine. With respect to the first sequence representation 226 and the second sequence representation, operation 230 can determine that the methylation states of the cytosines of the third CpG region 210 and the methylation states of cytosines for the fourth CpG region 212 are the same. Operation 230 can also determine that the methylations states of cytosines in the second CpG region 208 differ between the example first sequence representation 226 and the example second sequence representation 228.

At operation 238, sequence representations satisfying one or more classification criteria can be identified to determine a set of sequence representations 240 that are designated for further analysis. In one or more examples, the classification criteria can indicate a threshold amount of overlapping CpG regions between individual first sequence representations 216 and individual second sequence representations 220. In one or more illustrative examples, the threshold amount of overlapping CpG regions can include at least 1 overlapping CpG region between individual first sequence representations 216 and individual second sequence representations 220, at least 2 overlapping CpG regions between individual first sequence representations 216 and individual second sequence representations 220, at least 3 overlapping CpG regions between individual first sequence representations 216 and individual second sequence representations 220, at least 4 overlapping CpG regions between individual first sequence representations 216 and individual second sequence representations 220, at least 5 overlapping CpG regions between individual first sequence representations 216 and individual second sequence representations 220, at least 6 overlapping CpG regions between individual first sequence representations 216 and individual second sequence representations 220, at least 8 overlapping CpG regions between individual first sequence representations 216 and individual second sequence representations 220, or at least 10 overlapping CpG regions between individual first sequence representations 216 and individual second sequence representations 220. In one or more additional illustrative examples, the threshold amount of overlapping CpG regions can be from 1 overlapping CpG region to 25 overlapping CpG regions between individual first sequence representations 216 and individual second sequence representations 220, from 1 overlapping CpG region to 10 overlapping CpG regions between individual first sequence representations 216 and individual second sequence representations 220, from 1 overlapping CpG region to 5 overlapping CpG regions between individual first sequence representations 216 and individual second sequence representations 220, from 5 overlapping CpG regions to 10 overlapping CpG regions between individual first sequence representations 216 and individual second sequence representations 220, or from 10 overlapping CpG regions to 20 overlapping CpG regions between individual first sequence representations 216 and individual second sequence representations 220.

In one or more additional examples, the classification criteria can indicate a threshold number of cytosines between overlapping regions of individual first sequence representations 216 and individual second sequence representations 220 that have a same methylation state. For example, the classification criteria can indicate that methylation states of cytosines of at least 1 overlapping CpG region between individual first sequence representations 216 and individual second sequence representations 220 are the same, that methylation states of cytosines of at least 2 overlapping CpG regions between individual first sequence representations 216 and individual second sequence representations 220 are the same, that methylation states of cytosines of at least 3 overlapping CpG regions between individual first sequence representations 216 and individual second sequence representations 220 are the same, that methylation states of cytosines of at least 4 overlapping CpG regions between individual first sequence representations 216 and individual second sequence representations 220 are the same, that methylation states of cytosines of at least 5 overlapping CpG regions between individual first sequence representations 216 and individual second sequence representations 220 are the same, that methylation states of cytosines of at least 6 overlapping CpG regions between individual first sequence representations 216 and individual second sequence representations 220 are the same, that methylation states of cytosines of at least 8 overlapping CpG regions between individual first sequence representations 216 and individual second sequence representations 220 are the same, that methylation states of cytosines of at least 10 overlapping CpG regions between individual first sequence representations 216 and individual second sequence representations 220 are the same, or the methylation states of cytosines of at least 12 overlapping CpG regions between individual first sequence representations 216 and individual second sequence representations 220 are the same. In one or more illustrative examples, the classification criteria can indicate that methylation states of cytosines are the same in 1 overlapping CpG region to 30 overlapping CpG regions between individual first sequence representations 216 and individual second sequence representations 220, that methylation states of cytosines are the same in 1 overlapping CpG region to 20 overlapping CpG regions between individual first sequence representations 216 and individual second sequence representations 220, that methylation states of cytosines are the same in 1 overlapping CpG region to 10 overlapping CpG regions between individual first sequence representations 216 and individual second sequence representations 220, that methylation states of cytosines are the same from 1 overlapping CpG region to 5 overlapping CpG regions between individual first sequence representations 216 and individual second sequence representations 220, that methylation states of cytosines are the same in 5 overlapping CpG region to 10 overlapping CpG regions between individual first sequence representations 216 and individual second sequence representations 220, or that methylation states of cytosines are the same in 10 overlapping CpG regions to 20 overlapping CpG regions between individual first sequence representations 216 and individual second sequence representations 220.

In various examples, first sequence representations 216 that satisfy the classification criteria with respect to the second sequence representations 220 can be included in the set of sequence representations 240. In one or more additional examples, the first sequence representations 216 that satisfy the classification criteria with respect to the second sequence representations 220 can be excluded from the set of sequence representations 240. In these scenarios, the set of sequence representations 240 includes the first sequence representations 216 that do not satisfy the classification criteria with respect to the second sequence representations 220. In at least some examples, whether the first sequence representations 216 satisfying the classification criteria are included in the set of sequence representations 240 or excluded from the set of sequence representations 240 can be based on features of the subjects that provided the samples from which the second sequence representations 220 were derived. For example, in situations where the second sequence representations 220 correspond to samples derived from individuals in which cancer is not present, the first sequence representations 216 that satisfy the classification criteria with respect to the second sequence representations 220 are to be excluded from the set of sequence representations 240. In addition, in instances where the second sequence representations 220 correspond to samples derived from individuals in which cancer is present, the first sequence representations 216 that satisfy the classification criteria with respect to the second sequence representations 220 can be included in the set of sequence representations 240.

In one or more examples, the classification criteria can be applied to the example first sequence representation 226 and the example second sequence representation 228. In one or more further illustrative examples, a first classification criteria applied to the example first sequence representation 226 and the example second sequence representation 228 can indicate that at least 3 CpG regions overlap between the example first sequence representation 226 and the example second sequence representation 228 and a second classification criteria applied to the example first sequence representation 226 and the example second sequence representation 228 can indicate that methylation states of cytosines in at least 2 of the overlapping CpG regions are the same. Continuing with this example, since the example first sequence representation 226 and the example second sequence representation 228 have 3 overlapping CpG regions, the second CpG region 208, the third CpG region 210, and the fourth CpG region 212, the example first sequence representation 226 and the example second sequence representation 228 satisfy the first classification criteria. Additionally, since the methylation states of the cytosines in the third CpG region 210 and the fourth CpG region 212 between the example first sequence representation 226 and the example second molecule representation 228 are the same, the example first sequence representation 226 and the second sequence representation 228 satisfy the second classification criteria. As explained previously, whether the example first sequence representation 226 is included in the set of sequence representations 240 or excluded from the set of sequence representations 240 can be based on whether or not the samples used to produce the second sequence representations 220 are derived from individuals in which cancer is present or individuals in which cancer is not present.

In one or more examples, at operation 242, the set of sequence representations 240 can be analyzed to determine tumor information for a test subject that provided one or more samples from which the first sequence representations 216 were generated. In one or more illustrative examples, the tumor information can indicate at least one of a presence of cancer with respect to the test subject, a type of cancer present in the test subject, a probability of cancer being present in the test subject, a tumor fraction for the one or more samples provided by the test subject, a level of progression of cancer present in the test subject, or mutant allele fraction for the one or more samples provided by the test subject. In various examples, the tumor information can be determined at operation 242 by analyzing features of the set of sequence representations 240. In one or more illustrative examples, the sequence representations 240 that correspond to individual classification regions of the reference sequence 204.

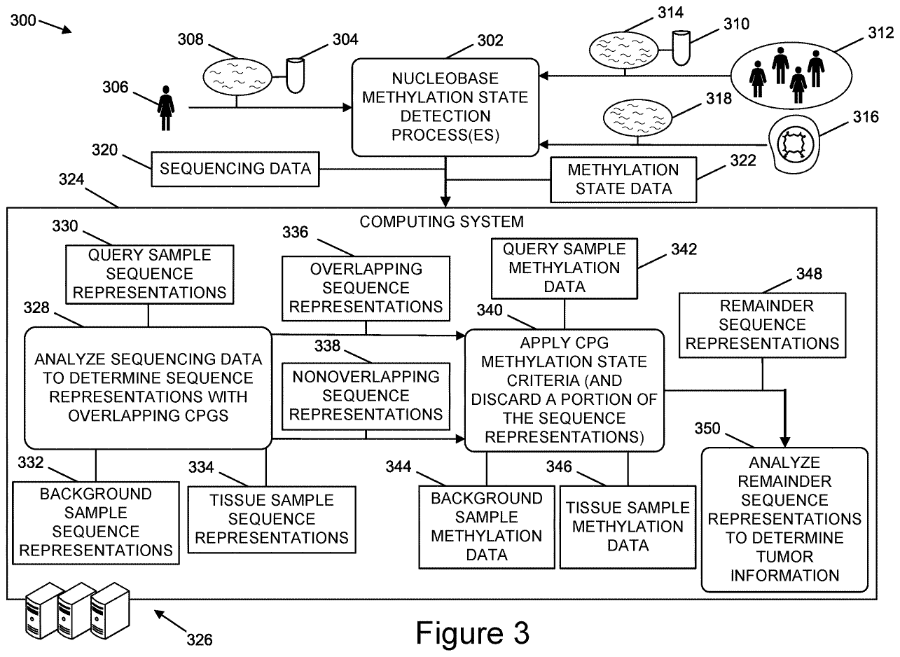

FIG. 3 is a diagrammatic representation of an example environment 300 to analyze sequence data derived from nucleic acid molecules obtained from a number of groups of subjects according to a number of classification criteria, according to one or more example implementations. The environment 300 can include one or more nucleobase methylation state detection processes 302. In one or more examples, the one or more nucleobase methylation state detection processes 102 can include at least one of sodium bisulfite conversion and sequencing, Tet-assisted bisulfite sequencing (TAB-Seq), differential enzymatic cleavage, one or more single molecule sequencing methods, such as nanopore DNA sequencing, oxidative bisulfite (Ox-BS) conversion, APOBEC-coupled epigenetic (ACE) conversion, or direct methylation sequencing (DM-Seq). The one or more nucleobase methylation state detection processes 302 can generate data indicating methylation states of nucleotides of nucleic acid molecules derived from a number of samples.

The one or more nucleobase methylation state detection processes 302 can be performed on a first sample 304 obtained from a first subject 306. First molecules 308 can be derived from the first sample 304. The first molecules 308 can be subjected to the one or more nucleobase methylation state detection processes 302 and the resulting data analyzed to determine tumor information about the first subject 306.

In addition, the one or more nucleobase methylation state detection processes 302 can be performed on a number of second samples 310. The number of second samples 310 can be obtained from second subjects 312. In one or more examples, the second subjects 312 can have a status of tumor not detected. That is, in at least some examples, the second subjects 312 can be cancer free. Second molecules 314 can be derived from the second samples 310 and the second molecules 314 can be subjected to the one or more nucleobase methylation state detection processes 302. In various examples, one or more of the second samples 310 can include one or more buffy coat samples derived from the second subjects 312. The one or more buffy coat samples can be separated from plasma and red blood cells through a centrifugation process. The one or more buffy coat samples can include at least one of lymphocytes, leukocytes, monocytes, granulocytes, thrombocytes, or platelets.

Further, the one or more nucleobase methylation state detection processes 302 can be performed on one or more third samples 316. The one or more third samples 316 can comprise one or more tissue samples. In one or more examples, the one or more third samples 316 can be obtained from one or more third subjects. In these scenarios, the one or more third samples 316 can include one or more tissue samples taken from one or more tumors of the one or more third subjects. In one or more additional examples, the one or more third samples 316 can include buffy coat derived from the one or more third subjects. In one or more additional examples, at least a portion of the one or more third samples 316 can be obtained from the first subject 306. In these situations, the one or more third samples 316 can include one or more tissue samples obtained from the first subject 306. Further, in instances where the one or more third samples 316 include buffy coat, in one or more implementations, at least a portion of the one or more third samples 316 can include buffy coat obtained from the first subject 306. In various examples, the one or more nucleobase methylation state detection processes 302 can correspond to the one or more nucleobase methylation state detection processes 102 described with respect to FIG. 1.

The one or more nucleobase methylation state detection processes 302 can generate sequencing data 320. For example, the one or more nucleobase methylation state detection processes 302 can include one or more sequencing operations. The sequencing data 320 can include a number of sequence representations that correspond to nucleotide sequences of the first molecules 308, the second molecules 314, and the third molecules 318. In at least some examples, the sequencing data 320 can include sequence representations of an amplification product produced by one or more sequencing operations included in the nucleobase methylation state detection processes 302.

The one or more nucleobase methylation state detection processes 302 can also produce methylation state data 322. The methylation state data 322 can indicate methylation states of a number of nucleotides for individual sequence representations included in the sequencing data 320. In one or more examples, the methylation state data 322 can indicate methylation states of cytosines of the sequence representations included in the sequencing data 320. In one or more illustrative examples, the methylation state data 322 can indicate locations of 5-methylcytosines in sequence representations included in the sequencing data 320.

The environment 300 can include a computing system 324 that analyzes the sequencing data 320 and the methylations state data 322. In various examples, the computing system 324 can perform at least a portion of the operations described with respect to FIG. 1 and FIG. 2. The computing system 324 can include one or more computing devices 326. The one or more computing devices 326 can include at least one of one or more desktop computing devices, one or more mobile computing devices, or one or more server computing device. In various examples, at least a portion of the one or more computing devices 326 can be included in a remote computing environment, such as a cloud computing environment. In one or more examples, the one or more nucleobase methylation state detection processes 302 and the operations executed by the computing system 324 can be performed by, controlled by, and/or maintained by a single organization. In one or more additional examples, the one or more nucleobase methylation state detection processes 302 and the operations executed by the computing system 324 can be performed by, controlled by, and/or maintained by multiple organizations.

In one or more examples, the computing system 324 can, at operation 328, analyze the sequencing data 320 to determine sequence representations with overlapping CpG regions. The computing system 324 can analyze query sample sequencing data 320 with respect to background sample sequence representations 332 and tissue sample sequence representations 334. The query sample sequencing data 320 can correspond to a first portion of the sequencing data 320 generated based on the first molecules 308 derived from the first sample 304 of the first subject 306. The background sample sequence representations 332 can correspond to a second portion of the sequencing data 320 generated based on the second molecules 314 derived from the second samples 310 of the second subjects 312. The tissue sample sequence representations 334 can correspond to a third portion of the sequencing data 320 generated based on the third molecules 318 derived from the third samples 316 of one or more third subjects.

The computing system 324 can determine overlapping CpG regions of the query sample sequence representations 330 in relation to the background sample sequence representations 332 and the tissue sample sequence representations 334 by aligning the sequence representations 330, 332, 334 to a reference sequence. After the alignment process, the computing system 324 can then determine query sample sequence representations 330 having CpG regions at genomic locations of the reference sequence that correspond to locations of the CpG regions of the background sample sequence representations 332 and the tissue sample sequence representations 334. In one or more examples, the computing system 324 can determine overlapping sequence representations 336 that include a first set of overlapping sequence representations that include query sample sequence representations 330 that have one or more CpG regions that overlap with corresponding CpG regions of the background sample sequence representations 332. Additionally, the computing system 324 can determine second overlapping sequence representations included in the overlapping sequence representations 336 that include query sample sequence representations 330 that have one or more CpG regions that overlap with corresponding CpG regions of the tissue sample sequence representations 334. Further, the computing system 324 can determine nonoverlapping sequence representations 338. The nonoverlapping sequence representations 338 can include query sample sequence representations 330 having less than a threshold number of CpG regions that overlap with the background sample sequence representations 332. In at least some examples, the computing system 324 can determine overlapping CpG regions and/or nonoverlapping CpG regions by implementing at least a portion of the techniques described with respect to FIG. 2.

In various examples, the computing system 324 can apply one or more classification criteria to determine the overlapping sequence representations 336 and the nonoverlapping sequence representations 338. For example, the computing system 324 can apply one or more classification criteria indicating that for query sample sequence representations 330 to be included in the overlapping sequence representations 336, at least a threshold number of CpG regions of individual query sample sequence representations 330 overlap with corresponding CpG regions of the background sample sequence representations 332. In addition, the computing system 324 can apply one or more classification criteria indicating that for query sample sequence representations 330 to be included in the overlapping sequence representations 336, at least a threshold number of CpG regions of individual query sample sequence representations 330 overlap with corresponding CpG regions of the tissue sample sequence representations 334. In one or more illustrative examples, the threshold number of overlapping CpG regions used by the computing system 324 to determine the first overlapping sequence representations can be different from the threshold number of overlapping CpG regions used by the computing system 324 to determine the second overlapping sequence representations. In one or more additional illustrative examples, the threshold number of overlapping CpG regions used by the computing system 324 to determine the first overlapping sequence representations can be the same as the threshold number of overlapping CpG regions used by the computing system 324 to determine the second overlapping sequence representations.

At operation 340, the computing system 324 can determine sequence representations with CpG methylations states satisfying one or more criteria. For example, the computing system 324 can analyze query sample methylation data 342 with respect to background sample methylation data 344 and tissue sample methylation data 346. The query sample methylation data 342 can comprise a first portion of the methylation state data 322 that corresponds to methylation states of cytosines of the first molecules 308 derived from the first sample 304 obtained from the first subject 306. The background sample methylation data 344 can comprise a second portion of the methylation state data 322 that corresponds to methylation states of cytosines of the second molecules 314 derived from the second samples 310 obtained from the second subjects 312. Further, the tissue sample methylation data 346 can comprise a third portion of the methylation state data 322 that corresponds to methylation states of cytosines of the third molecules 318 derived from the third samples 316 obtained from one or more third subjects.

In one or more examples, at operation 340, the computing system 324 can analyze methylation states of CpG regions of the overlapping sequence representations 336 with respect to one or more criteria. In various examples, the computing system 324 can analyze the query sample methylation data 342 and the background sample methylation data 344 with respect to the first overlapping sequence representations. For example, the computing system 324 can analyze methylation states of cytosines of individual overlapping CpG regions with respect to query sample sequence representations 330 and the background sample sequence representations 332. In one or more additional examples, the computing system 324 can analyze the query sample methylation data 342 and the tissue sample methylation data 346 with respect to the second overlapping sequence representations. To illustrate, the computing system 324 can analyze methylation states of cytosines of individual overlapping CpG regions with respect to the query sample sequence representations and the tissue sample sequence representations 334.

The one or more criteria implemented by the computing system 324 at operation 340 can indicate a threshold number of overlapping CpG regions having cytosines with corresponding methylation states. In one or more illustrative examples, the computing system 324 can, for an individual overlapping CpG region, analyze the methylation states of the individual cytosines of a query sample sequence representation 330 in the overlapping CpG region and the methylation states of the individual cytosines of a background sample sequence representation 332 in the overlapping CpG region. In one or more additional illustrative examples, the computing system 324 can, for an additional overlapping CpG region, analyze the methylation states of the individual cytosines of a query sample sequence representation 330 in the additional overlapping CpG region and the methylation states of the tissue sample sequence representations 334 in the overlapping CpG region. In one or more examples, the computing system 324 can determine that overlapping CpG regions have corresponding methylation states or matching methylation states when at least 75% of the cytosines have the same methylation state, when at least 80% of the cytosines have the same methylation state, when at least 85% of the cytosines have the same methylation state, when at least 90% of the cytosines have the same methylation state, when at least 95% of the cytosines have the same methylation state, when at least 99% of the cytosines have the same methylation state, or when all of the cytosines have the same methylation state.

The computing system 324 can generate remainder sequence representations 348. The remainder sequence representations 348 can include at least a portion of the query sample sequence representations 330 having at least a threshold number of overlapping CpG regions with at least an additional threshold number of the overlapping CpG regions and having corresponding methylation states with one or more tissue sample sequence representations 334. In at least some examples, the remainder sequence representations 348 can also include at least a portion of the nonoverlapping sequence representations 338 that include one or more query sample sequence representations 330 that do not satisfy overlapping criteria with respect to at least one background sample sequence representation 332.

In various examples, the computing system 324, at operation 340, can discard from further analysis at least a portion of the query sample sequence representations 330 that are included in the nonoverlapping sequence representations 338. For example, the computing system 324 can discard query sample sequence representations 330 that do not satisfy one or more CpG region overlapping criteria with respect to the tissue sample sequence representations 334. Additionally, the computing system 324 can discard from further analysis a portion of the query sample sequence representations 330 that satisfy the one or more CpG region overlapping criteria with respect to the background sample sequence representations 332 and that satisfy the one or more methylation state criteria.

In still other examples, at operation 340, the computing system 324 can apply one or more additional criteria to generate the remainder sequence representations 348. To illustrate, the computing system 324 can apply a criteria corresponding to a threshold number of a same query sample sequence representation 330 overlapping with a given tissue sample sequence representation 334 before including the given query sample sequence representation 330 in the remainder sequence representations 348. In one or more examples, for an individual query sample sequence representation multiple copies of the individual query sample sequence representation can be present in the query sample sequence representations 330 or a single copy of the individual query sample sequence representation can be present in the query sample sequence representations 330. The computing system 324 can analyze a number of copies of an individual query sample sequence representation that overlap with a given tissue sample sequence representation 334. In situations where the number of copies of the individual query sample sequence representation is at least a threshold number, the query sample sequence representation can be included in the remainder sequence representations 348 and further analyzed by the computing system 324. In scenarios where the number of copies of the individual query sample sequence representation is less than the threshold number, the query sample sequence representation can be excluded from the remainder sequence representations 348.

The computing system, at operation 350, can analyze the remainder sequence representations 348 to determine tumor information about the first subject 306. In one or more illustrative examples, the tumor information can indicate at least one of a presence of cancer with respect to the first subject 306, a type of cancer present in the first subject 306, a probability of cancer being present in the first subject 306, a tumor fraction for the one or more samples provided by the first subject 306, a level of progression of cancer present in the first subject 306, or mutant allele fraction for the one or more samples provided by the first subject 306.

To determine the tumor information, the computing system 324 can analyze a number of the remainder sequence representations 348 that correspond to one or more classification regions of a reference sequence. In one or more examples, a quantitative measure corresponding to a classification region metric can be determined that indicates a number of the remainder sequence representations 348 that correspond to individual classification regions in relation to a total number of molecules derived from the first sample 304, the second samples 310, and the one or more third samples 316. In at least some examples, one or more statistical operations can be performed using the quantitative measures to determine the tumor information. In still other examples, individual quantitative measures for a first number of classification regions can be weighted differently than individual quantitative measures for a second number of classification regions. In one or more illustrative examples, individual quantitative measures can be used to determine the tumor information based on the corresponding classification region being associated with at least a threshold number of the remainder sequence representations 348. In one or more additional illustrative examples, the tumor information can be determined using quantitative measures related to classification regions that minimize a noise signal with respect to information indicated by the remainder sequence representations 348. In one or more further illustrative examples, the computing system 324 can analyze remainder sequence representations 348 that correspond to from 50 classification regions to 5000 classification regions, from 100 classification regions to 4000 classification regions, from 500 classification regions to 3000 classification regions, from 1000 classification regions to 3000 classification regions, from 1500 classification regions to 3000 classification regions, from 2000 classification regions to 3000 classification regions, from 100 classification regions to 1000 classification regions, from 500 classification regions to 1500 classification regions, or from 1000 classification regions to 2000 classification regions.

In one or more examples, the computing system 324 can determine tumor information for a type of cancer. For example, the one or more third samples 316 can be obtained from individuals in which a single type of cancer is present. In these scenarios, the computing system 324 can determine tumor information related to the single type of cancer. In one or more additional examples, the one or more third samples 316 can be obtained from individuals in which a number of different types of cancer are present. In these situations, the computing system 324 can determine tumor information for at least a portion of the different types of cancer. In one or more illustrative examples, the computing system 324 can determine overlapping sequence representations, nonoverlapping sequence representations, and apply methylation state criteria to portions of the tissue sample sequence representations 334 that correspond to individual cancer types. In this way, the operations 328, 340, and 350 can be performed multiple times with each combination of operations 328, 340, and 350 being performed for an individual cancer type. In one or more further examples, the computing system 324 can determine tumor information related to one or more stages of one or more types of cancer.

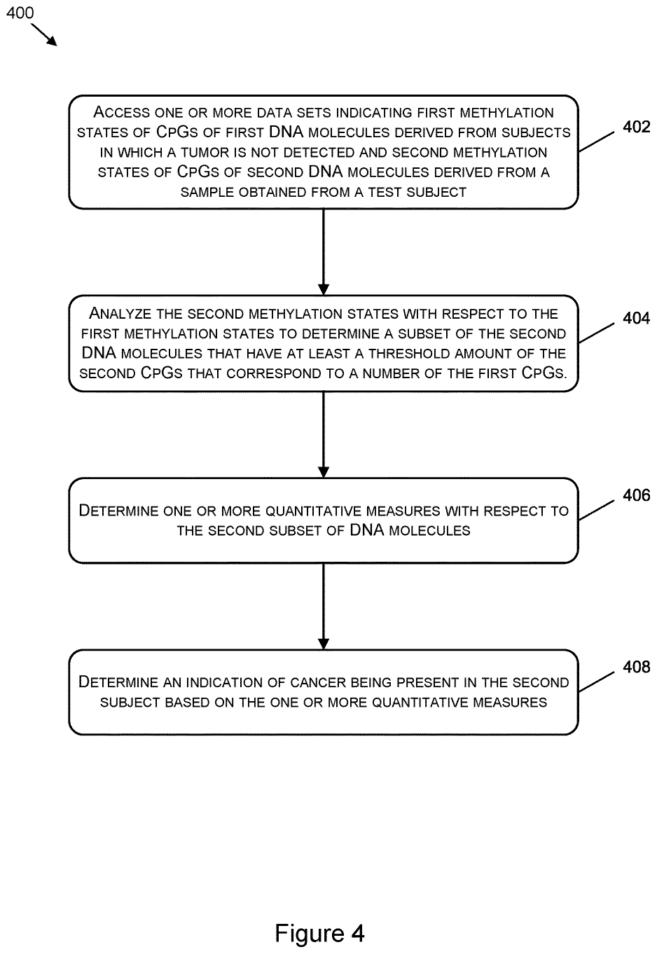

FIG. 4 is a flow diagram of an example process to determine an indication of cancer being present in a test subject by analyzing methylation states of nucleic acid molecules derived from the test subject and derived from one or more groups of subjects, according to one or more implementations. At operation 402, the method 400 can include accessing one or more data sets indicating first methylation states of individual cytosine nucleotides included in first cytosine-guanine dinucleotides (CpGs) of first deoxyribonucleic acid (DNA) molecules in a first dataset. In at least some examples, the first dataset can include sequence representations that correspond to the first DNA molecules. The first DNA molecules related to the first dataset can be derived from first samples obtained from first subjects in which a tumor is not detected. The one or more datasets can also indicate second methylation states of individual cytosine nucleotides included in second CpGs of second DNA molecules in a second dataset. In various examples, the second dataset can include sequence representations that correspond to the second DNA molecules. The second DNA molecules related to the second dataset can be derived from one or more second samples obtained from a second subject. In one or more examples, the second subject can be a test subject for which tumor information is being determined. In one or more illustrative examples, the first dataset and the second dataset can be combined into a single dataset. In one or more additional illustrative examples, the first DNA molecules can include at least one of: cell-free DNA molecules derived from one or more plasma samples obtained from the first subjects, buffy coat derived from the first subjects, or DNA molecules obtained from tissue samples extracted from the first subjects or DNA molecules obtained from tissue samples extracted from the first subjects. In one or more further illustrative examples, the second DNA molecules can include at least one of cell-free DNA molecules derived from one or more plasma samples obtained from the second subject, buffy coat derived from the second subject, or DNA molecules obtained from a tissue sample extracted from the second subject.

In addition, the method 400 can include, at operation 404, analyzing the second methylation states of the second CpGs with respect to the first methylation states of the first CpGs to determine a subset of the second DNA molecules. Individual second DNA molecules of the subset of second molecules can have at least a threshold amount of the second CpGs that correspond to a number of the first CpGs. In at least some examples, the group of the second DNA molecules that includes the additional subset of the second DNA molecules can be determined by determining that a number of the additional subset of the second DNA molecules is at least an additional threshold number.

In various examples, the method 400 can include determining sequence representations of DNA molecules to include in an analysis to determine tumor information for the second subject and determining sequence representations of DNA molecules to exclude from the analysis to determine tumor information for the second subject. For example, a group of the second DNA molecules can be determined that excludes the subset of the second DNA molecules and includes the additional subset of the second DNA molecules. Additionally, determining the subset of second DNA molecules can comprise determining an individual second DNA molecule having a plurality of CpGs with genomic locations that align with a plurality of additional CpGs at the genomic locations of an individual first DNA molecule. Further, determining the subset of second DNA molecules can also comprise determining an individual second DNA molecule having a plurality of CpGs with genomic locations that align with a plurality of additional CpGs at the genomic locations of an individual first DNA molecule. In still other examples, determining the subset of second DNA molecules can further include determining that a number of the plurality of CpGs of the individual second DNA molecule having methylation states of individual cytosines that are the same as the additional methylation states of the individual additional cytosines of the plurality of additional CpGs of the individual first DNA molecule is at least a threshold number. In these scenarios, the individual second DNA molecule is to be excluded from the group of second DNA molecules.

In one or more illustrative examples, determining the group of the second DNA molecules that excludes the subset of the second DNA molecules and includes the additional subset of the second DNA molecules can comprise determining an individual second DNA molecule having a plurality of CpGs with genomic locations that align with a plurality of additional CpGs at the genomic locations of an individual first DNA molecule. In addition, determining the group of the second DNA molecules that excludes the subset of the second DNA molecules and includes the additional subset of the second DNA molecules can also comprise determining that a number of the plurality of CpGs of the individual second DNA molecule having methylation states of individual cytosines that are different from the additional methylation states of the individual additional cytosines of the plurality of additional CpGs of the individual third DNA molecule is at least a threshold number. In still other examples, determining the group of the second DNA molecules that excludes the subset of the second DNA molecules and includes the additional subset of the second DNA molecules can further comprise determining that the individual second DNA molecule is to be included in an additional group of DNA molecules that is analyzed with respect to the third data set.

In one or more further examples, a determination can be made that the individual second DNA molecule has a plurality of CpGs with genomic locations that align with a plurality of additional CpGs at the genomic locations of an individual third DNA molecule. An additional determination can be made that a number of the plurality of CpGs of the individual second DNA molecule having methylation states of individual cytosines that are the same as the additional methylation states of the individual additional cytosines of the plurality of additional CpGs of the individual third DNA molecule is at least a threshold number. As a result of these operations, the individual second DNA molecule is to be included in the group of second DNA molecules. Determining that the individual second DNA molecule is to be included in the group of second DNA molecules can also comprise determining that a number of first DNA molecules that are matched with an individual second molecule and if that number is greater than a threshold then that individual second molecule is excluded from the subset of the second molecule.

In at least some examples, determining that the individual second DNA molecule has a plurality of CpGs with genomic locations that align with the plurality of additional CpGs at the genomic locations of the individual first DNA molecule can comprise analyzing individual positions of individual CpGs of the second DNA molecule with respect to individual additional positions of a plurality of additional CpGs of a plurality of first DNA molecules to determine one or more DNA molecule pairings. An individual DNA molecule pairing can include the second DNA molecule and a first DNA molecule having at least a threshold number of aligned CpGs. Additionally, an aligned CpG can corresponds to a position of a reference genome where a CpG of the second molecule is located and a CpG of the first molecule is located. Additionally, analyzing the second methylation states of the second CpGs with respect to the first methylation states of the first CpGs to determine the subset of the second DNA molecules can comprise determining that a methylation state of a CpG of the second molecule corresponds to an additional methylation state of an additional CpG of a respective first molecule for at least a threshold number of aligned CpGs of the second molecule and the respective first molecule.

In addition, determining that the individual second DNA molecule has a plurality of CpGs with genomic locations that align with the plurality of additional CpGs at the genomic locations of the individual third DNA molecule can comprise analyzing individual positions of individual CpGs of the second DNA molecule with respect to individual additional positions of a plurality of additional CpGs of a plurality of third DNA molecules to determine one or more DNA molecule pairings. In these scenarios, an individual DNA molecule pairing can include the second DNA molecule and a third DNA molecule having at least a threshold number of aligned CpGs. Also in these situations, an aligned CpG corresponds to a position of a reference genome where a CpG of the second molecule is located and a CpG of the first molecule is located. Further, analyzing the second methylation states of the second CpGs with respect to the third methylation states of the third CpGs to determine the additional subset of the second DNA molecules can comprise determining that a methylation state of a CpG of the second molecule corresponds to an additional methylation state of an additional CpG of a respective third molecule for at least at threshold number of aligned CpGs of the second molecule and the respective third molecule.

In one or more examples, additional datasets can be analyzed. For example, a third data set can be accessed indicating third methylation states of individual cytosine nucleotides included in third CpGs of third DNA molecules. In at least some examples, the third dataset can include sequence representations that correspond to the third DNA molecules. The third DNA molecules of the third dataset can be derived from one or more third samples obtained from one or more third subjects in which a tumor is detected. In addition, the one or more third samples can include one or more tissue samples obtained from the one or more third subjects. In one or more illustrative examples, the one or more third subjects can include the second subject. In at least some examples, the third DNA molecules can comprise at least one of cell-free DNA molecules derived from one or more plasma samples obtained from the third subjects or DNA molecules obtained from tissue samples extracted from the third subjects.

In at least some examples, at least a portion of the first methylation states can indicate first methylated cytosines of the first CpGs, at least a portion of the second methylation states can indicate second methylated cytosines of the second CpGs, and at least a portion of the third methylation states indicate third methylated cytosines of the third CpGs. In one or more additional examples, at least a portion of the first methylation states can indicate first unmethylated cytosines of the first CpGs, at least a portion of the first methylation states indicate first unmethylated cytosines of the first CpGs, and at least a portion of the third methylation states indicate third unmethylated cytosines of the third CpGs.

In one or more additional examples, at least one of the first CpGs, the second CpGs, or the third CpGs can be located in a plurality of classification regions. In one or more illustrative examples, the plurality of classification regions can include CpGs having a first methylation state in subjects in which cancer is present and a second methylation state different from the first methylation state in additional subjects in which cancer is not present. In at least some examples, at least a threshold number of at least one of the first DNA molecules, the second DNA molecules, or the third DNA molecules can be identified that correspond to a subset of the plurality of classification regions. In these situations, at least one of the first CpGs, the second CpGs, or the third CpGs are located in the subset of the plurality of classification regions.

In various examples, the second methylation states of the second CpGs can be analyzed with respect to the third methylation states of the third CpGs to determine an additional subset of the second DNA molecules. Additional individual second DNA molecules of the additional subset of the second molecules can have at least the threshold amount of the second CpGs that correspond to the third CpGs.

In one or more further examples, a further data set can be accessed. The further data set can indicate fourth methylation states of individual cytosine nucleotides included in fourth CpGs of fourth DNA molecules. The fourth DNA molecules can be derived from one or more fourth samples obtained from one or more fourth subjects in which a tumor corresponding to an additional type of cancer is detected. In these instances, the second methylation states of the second CpGs can be analyzed with respect to the fourth methylation states of the fourth CpGs to determine a further subset of the second DNA molecules. Further individual second DNA molecules of the further subset of the second DNA molecules can have at least the threshold amount of the second CpGs that correspond to the fourth CpGs. In addition, one or more quantitative measures can be determined with respect to the subset of the second DNA molecules and the further subset of the second DNA molecules. Also, an additional indication of the additional type of cancer being present in the second subject can be determined based on the one or more additional quantitative measures. In one or more illustrative examples, the one or more fourth samples can include at least one of cell-free DNA molecules derived from one or more plasma samples obtained from the fourth subjects or DNA molecules obtained from tissue samples extracted from the fourth subjects.

In still other examples, buffy coat samples can be analyzed to determine the subset of the second DNA molecules. In one or more examples, one or more buffy coat samples can be obtained from the second subject. The one or more buffy coat samples can correspond to a data set indicating methylation states of individual cytosine nucleotides included in CpGs of DNA molecule sequence representations derived from the one or more buffy coat samples obtained from second subject. The second methylation states of the second CpGs obtained from a plasma sample of the second subject can be analyzed with respect to the methylation states of the CpGs included in the buffy coat molecule sequence representations to determine the subset of the second DNA molecule sequence representations. In one or more examples, methylation states of at least a portion of the second DNA molecule sequence representations derived from a plasma sample of the second subject can be analyzed with respect to methylation states of DNA molecule sequence representations derived from one or more buffy coat samples of the second subject. In scenarios where methylation states of DNA molecule sequence representations derived from a plasma sample of the second subject corresponds to methylation states of DNA molecule sequence representations derived from a buffy coat sample of the subject, the DNA molecule sequence representations derived from the plasma sample of the second subject can be excluded from the subset of the second DNA molecule sequence representations.

In one or more illustrative examples, an individual DNA molecule sequence representation derived from a plasma sample of the second subject can be identified that has a plurality of CpGs with genomic locations aligned with a plurality of CpGs at the genomic locations with respect to a DNA molecule sequence representations derived from a buffy coat sample of the second subject. For the aligned DNA molecule sequence representation derived from the plasma sample of the second subject, a number of the CpGs of the aligned molecule sequence representation having a same methylation state as the corresponding CpGs of the buffy coat derived molecule sequence representation can be determined. In scenarios where the number of aligned CpGs having the same methylation state is greater than a threshold number, the individual DNA molecule sequence representation derived from the plasma sample of the second subject can be excluded from the subset of the second DNA molecule sequence representations.

In one or more additional examples, one or more buffy coat samples can be obtained from the at least a portion of the first subjects in which a tumor is not detected. The one or more buffy coat samples can correspond to a data set indicating methylation states of individual cytosine nucleotides included in CpGs of DNA molecule sequence representations derived from the one or more buffy coat samples obtained from the at least a portion of the first subjects. The second methylation states of the second CpGs obtained from a plasma sample of the second subject can be analyzed with respect to the methylation states of the CpGs included in the buffy coat molecule sequence representations to determine the subset of the second DNA molecule sequence representations. In one or more examples, methylation states of at least a portion of the second DNA molecule sequence representations derived from a plasma sample of the second subject can be analyzed with respect to methylation states of DNA molecule sequence representations derived from one or more buffy coat samples of at least a portion of the second subjects. In scenarios where methylation states of DNA molecule sequence representations derived from a plasma sample of the second subject corresponds to methylation states of DNA molecule sequence representations derived from a buffy coat sample of one or more first subjects, the DNA molecule sequence representations derived from the plasma sample of the second subject can be excluded from the subset of the second DNA molecule sequence representations.

In one or more additional illustrative examples, an individual DNA molecule sequence representation derived from a plasma sample of the second subject can be identified that has a plurality of CpGs with genomic locations aligned with a plurality of CpGs at the genomic locations with respect to a DNA molecule sequence representations derived from a buffy coat sample of one or more first subjects. For the aligned DNA molecule sequence representation derived from the plasma sample of the second subject, a number of the CpGs of the aligned molecule sequence representation having a same methylation state as the corresponding CpGs of the buffy coat derived molecule sequence representation can be determined. In scenarios where the number of aligned CpGs having the same methylation state is greater than a threshold number, the individual DNA molecule sequence representation derived from the plasma sample of the second subject can be excluded from the subset of the second DNA molecule sequence representations.

The method 400 can also include, at operation 406, determining one or more quantitative measures with respect to the subset of the second DNA molecules. In one or more examples, a number of second molecules included in the group of the second molecules can be determined. In addition, an aggregate number of second molecules included in the second data set can also be determined. In these scenarios, the one or more quantitative measures can correspond to a proportion of the number of second molecules in relation to the aggregate number of second molecules. In one or more additional examples, the one or more quantitative measures include counts of the second molecules included in the group of the second molecules.

Further, at operation 408, the method 400 can include determining an indication of cancer being present in the second subject based on the one or more quantitative measures. In one or more examples, a type of cancer present in the second subject can correspond to a type of cancer present in one or more additional subjects that provided samples. A same type of cancer can be present in the one or more additional subjects. In one or more additional examples, the indication of cancer being present in the second subject can correspond to tumor fraction. In one or more illustrative examples, the tumor fraction can be analyzed to determine a stage of cancer present in the second subject.

Although the illustrative example method 400 is described with respect to determining quantitative measures and an indication of cancer with respect to DNA molecules data, the method 400 can also be performed with respect to sequence representations. The sequence representations can correspond to sequencing reads derived from sequencing the various DNA molecules. The sequence representations can also correspond to nucleotide sequences of the DNA molecules themselves.

### Exemplary Methods

### A. Determining an Indication of Cancer in a Sample

A method includes accessing one or more data sets indicating first methylation states of individual cytosine nucleotides included in first cytosine-guanine dinucleotides (CpGs) of first deoxyribonucleic acid (DNA) molecule sequence representations in a first dataset derived from first samples obtained from first subjects in which a tumor is not detected. The one or more datasets may also include second methylation states of individual cytosine nucleotides included in second CpGs of second DNA molecule sequence representations in a second dataset derived from one or more second samples obtained from a second subject. The method may also include analyzing the second methylation states of the second CpGs with respect to the first methylation states of the first CpGs to determine a subset of the second DNA molecule sequence representations. Individual second DNA molecule sequence representations of the subset of the second DNA molecule sequence representations may have at least a threshold amount of the second CpGs that correspond to a number of the first CpGs. In addition, the method may include determining one or more quantitative measures with respect to the subset of the second DNA molecule sequence representations. Further, the method may include determining an indication of cancer being present in the second subject based on the one or more quantitative measures.

A computing apparatus includes a processor and memory storing instructions that, when executed by the processor, configure the apparatus to access one or more data sets. The one or more datasets may indicate first methylation states of individual cytosine nucleotides included in first cytosine-guanine dinucleotides (CpGs) of first deoxyribonucleic acid (DNA) molecule sequence representations in a first dataset derived from first samples obtained from first subjects in which a tumor is not detected. The one or more datasets may also include second methylation states of individual cytosine nucleotides included in second CpGs of second DNA molecule sequence representations in a second dataset derived from one or more second samples obtained from a second subject. The memory may also store instructions that configure the computing apparatus to analyze the second methylation states of the second CpGs with respect to the first methylation states of the first CpGs to determine a subset of the second DNA molecule sequence representations. Individual second DNA molecule sequence representations of the subset of the second DNA molecule sequence representations having at least a threshold amount of the second CpGs that correspond to a number of the first CpGs. In addition, the memory may store instructions that configure the computing apparatus to determine one or more quantitative measures with respect to the subset of the second DNA molecule sequence representations. Further, the memory may also store instructions that configure the computing apparatus to determine an indication of cancer being present in the second subject based on the one or more quantitative measures.

In one aspect, a non-transitory computer-readable storage medium, includes instructions that when executed by a computer, cause the computer to access one or more data sets. The one or more datasets may indicate first methylation states of individual cytosine nucleotides included in first cytosine-guanine dinucleotides (CpGs) of first deoxyribonucleic acid (DNA) molecule sequence representations in a first dataset derived from first samples obtained from first subjects in which a tumor is not detected. The one or more datasets may also indicate second methylation states of individual cytosine nucleotides included in second CpGs of second DNA molecule sequence representations in a second dataset derived from one or more second samples obtained from a second subject. The non-transitory computer-readable storage medium may also include instructions that when executed by the computer, cause the computer to analyze the second methylation states of the second CpGs with respect to the first methylation states of the first CpGs to determine a subset of the second DNA molecule sequence representations, individual second DNA molecule sequence representations of the subset of the second DNA molecule sequence representations having at least a threshold amount of the second CpGs that correspond to a number of the first CpGs. In addition, the non-transitory computer-readable storage medium may include instructions that when executed by the computer, cause the computer to determine one or more quantitative measures with respect to the subset of the second DNA molecule sequence representations. Further, the non-transitory computer-readable storage medium may include instructions that when executed by the computer, cause the computer to and determine an indication of cancer being present in the second subject based on the one or more quantitative measures.

### B. Adapter Ligation

In some embodiments, adapters are added to the DNA. This may be done concurrently with an amplification procedure, e.g., by providing the adapters in a 5′ portion of a primer (where PCR is used, this can be referred to as library prep-PCR or LP-PCR). In some embodiments, adapters are added by other approaches, such as ligation. In some such methods, prior to partitioning or prior to capturing, first adapters are added to the nucleic acids by ligation to the 3′ ends thereof, which may include ligation to single-stranded DNA. The adapter can be used as a priming site for second-strand synthesis, e.g., using a universal primer and a DNA polymerase. A second adapter can then be ligated to at least the 3′ end of the second strand of the now double-stranded molecule. In some embodiments, the first adapter comprises an affinity tag, such as biotin, and nucleic acid ligated to the first adapter is bound to a solid support (e.g., bead), which may comprise a binding partner for the affinity tag such as streptavidin. For further discussion of a related procedure, see Gansauge et al., Nature Protocols 8:737-748 (2013). Commercial kits for sequencing library preparation compatible with single-stranded nucleic acids are available, e.g., the Accel-NGS® Methyl-Seq DNA Library Kit from Swift Biosciences. In some embodiments, after adapter ligation, nucleic acids are amplified.

Preferably, the adapters include different tags of sufficient numbers that the number of combinations of tags results in a low probability e.g., 95, 99 or 99.9% of two nucleic acids with the same start and stop points receiving the same combination of tags. Adapters, whether bearing the same or different tags, can include the same or different primer binding sites, but preferably adapters include the same primer binding site.

In some embodiments, following attachment of adapters, the nucleic acids are subject to amplification. The amplification can use, e.g., universal primers that recognize primer binding sites in the adapters.

In some embodiments, following attachment of adapters, the DNA is partitioned, comprising contacting the DNA with an agent that preferentially binds to nucleic acids bearing an epigenetic modification. The nucleic acids are partitioned into at least two subsamples differing in the extent to which the nucleic acids bear the modification from binding to the agents. For example, if the agent has affinity for nucleic acids bearing the modification, nucleic acids overrepresented in the modification (compared with median representation in the population) preferentially bind to the agent, whereas nucleic acids underrepresented for the modification do not bind or are more easily eluted from the agent. The nucleic acids can then be amplified from primers binding to the primer binding sites within the adapters. Partitioning may be performed instead before adapter attachment, in which case the adapters may comprise differential tags that include a component that identifies which partition a molecule occurred in. In some embodiments, the nucleic acids are linked at both ends to Y-shaped adapters including primer binding sites and tags. The molecules are amplified.

### C. Tagging

“Tagging” DNA molecules is a procedure in which a tag is attached to or associated with the DNA molecules. Tags can be molecules, such as nucleic acids, containing information that indicates a feature of the molecule with which the tag is associated. For example, molecules can bear a sample tag (which distinguishes molecules in one sample from those in a different sample) or a molecular tag/molecular barcode/barcode (which distinguishes different molecules from one another (in both unique and non-unique tagging scenarios). For methods that involve a partitioning step, a partition tag (which distinguishes molecules in one partition from those in a different partition) may be included. In some embodiments, adapters added to DNA molecules comprise tags. In certain embodiments, a tag can comprise one or a combination of barcodes. As used herein, the term “barcode” refers to a nucleic acid molecule having a particular nucleotide sequence, or to the nucleotide sequence, itself, depending on context. A barcode can have, for example, between 10 and 100 nucleotides. A collection of barcodes can have degenerate sequences or can have sequences having a certain hamming distance, as desired for the specific purpose. So, for example, a molecular barcode can be comprised of one barcode or a combination of two barcodes, each attached to different ends of a molecule. Additionally, or alternatively, for different partitions and/or samples, different sets of molecular barcodes, or molecular tags can be used such that the barcodes serve as a molecular tag through their individual sequences and also serve to identify the partition and/or sample to which they correspond based the set of which they are a member.

In some embodiments, two or more partitions, e.g., each partition, is/are differentially tagged. Tags can be used to label the individual polynucleotide population partitions so as to correlate the tag (or tags) with a specific partition. Alternatively, tags can be used in embodiments that do not employ a partitioning step. In some embodiments, a single tag can be used to label a specific partition. In some embodiments, multiple different tags can be used to label a specific partition. In embodiments employing multiple different tags to label a specific partition, the set of tags used to label one partition can be readily differentiated for the set of tags used to label other partitions. In some embodiments, the tags may have additional functions, for example the tags can be used to index sample sources or used as unique molecular identifiers (which can be used to improve the quality of sequencing data by differentiating sequencing errors from mutations, for example as in Kinde et al., Proc Nat'l Acad Sci USA 108:9530-9535 (2011), Kou et al., PLOS ONE, 11: e0146638 (2016)) or used as non-unique molecule identifiers, for example as described in U.S. Pat. No. 9,598,731. Similarly, in some embodiments, the tags may have additional functions, for example the tags can be used to index sample sources or used as non-unique molecular identifiers (which can be used to improve the quality of sequencing data by differentiating sequencing errors from mutations). In some embodiments, partition tagging comprises tagging molecules in each partition with a partition tag. After re-combining partitions (e.g., to reduce the number of sequencing runs needed and avoid unnecessary cost) and sequencing molecules, the partition tags identify the source partition. In some embodiments, the partition tags can serve as identifiers of the source partition and the molecule, i.e., different partitions are tagged with different sets of molecular tags, e.g., comprised of a pair of barcodes. In this way, the one or more molecular barcodes attached to the molecule indicates the source partition as well as being useful to distinguish molecules within a partition. For example, a first set of 35 barcodes can be used to tag molecules in a first partition, while a second set of 35 barcodes can be used tag molecules in a second partition.

In some embodiments, after partitioning and tagging with partition tags, the molecules may be pooled for sequencing in a single run. In some embodiments, a sample tag is added to the molecules, e.g., in a step subsequent to addition of partition tags and pooling. Sample tags can facilitate pooling material generated from multiple samples for sequencing in a single sequencing run.

Alternatively, in some embodiments, partition tags may be correlated to the sample as well as the partition. As a simple example, a first tag can indicate a first partition of a first sample; a second tag can indicate a second partition of the first sample; a third tag can indicate a first partition of a second sample; and a fourth tag can indicate a second partition of the second sample.

While tags may be attached to molecules already partitioned based on one or more characteristics, the final tagged molecules in the library may no longer possess that characteristic. For example, while single stranded DNA molecules may be partitioned and tagged, the final tagged molecules in the library are likely to be double stranded. Similarly, while DNA may be subject to partition based on different levels of methylation, in the final library, tagged molecules derived from these molecules are likely to be unmethylated. Accordingly, the tag attached to molecule in the library typically indicates the characteristic of the “parent molecule” from which the ultimate tagged molecule is derived, not necessarily to characteristic of the tagged molecule, itself.

As an example, barcodes 1, 2, 3, 4, etc. are used to tag and label molecules in the first partition; barcodes A, B, C, D, etc. are used to tag and label molecules in the second partition; and barcodes a, b, c, d, etc. are used to tag and label molecules in the third partition. Differentially tagged partitions can be pooled prior to sequencing. Differentially tagged partitions can be separately sequenced or sequenced together concurrently, e.g., in the same flow cell of an Illumina sequencer.

After sequencing, analysis of reads can be performed on a partition-by-partition level, as well as a whole DNA population level. Tags are used to sort reads from different partitions. Analysis can include in silico analysis to determine genetic and epigenetic variation (one or more of methylation, chromatin structure, etc.) using sequence information, genomic coordinates length, coverage, and/or copy number. In some embodiments, higher coverage can correlate with higher nucleosome occupancy in genomic region while lower coverage can correlate with lower nucleosome occupancy or a nucleosome depleted region (NDR).

### D. Enriching/Capturing Step; Amplification

Methods disclosed herein can comprise capturing DNA, such as cfDNA target regions. In some embodiments, the capturing comprises contacting the DNA with probes (e.g., oligonucleotides) specific for the target regions. Enrichment or capture may be performed on any sample or subsample described herein using any suitable approach known in the art.

In some embodiments, enrichment or capture is performed after attachment of adapters to sample molecules. In some embodiments, enrichment or capture is performed after a partitioning step. In some embodiments, enrichment or capture is performed after an amplification step. In some embodiments, sample molecules are partitioned, then adapters are attached, then sample molecules are amplified, and then the amplified molecules are subjected to enrichment or capture. The enriched or captured molecules may then be subjected to another amplification and then sequenced.

In some embodiments, the probes specific for the target regions comprise a capture moiety that facilitates the enrichment or capture of the DNA hybridized to the probes. In some embodiments, the capture moiety is biotin. In some such embodiments, streptavidin attached to a solid support, such as magnetic beads, is used to bind to the biotin. Nonspecifically bound DNA that does not comprise a target region is washed away from the captured DNA. In some embodiments, DNA is then dissociated from the probes and eluted from the solid support using salt washes or buffers comprising another DNA denaturing agent. In some embodiments, the probes are also eluted from the solid support by, e.g., disrupting the biotin-streptavidin interaction. In some embodiments, captured DNA is amplified following elution from the solid support. In some such embodiments, DNA comprising adapters is amplified using PCR primers that anneal to the adapters. In some embodiments, captured DNA is amplified while attached to the solid support. In some such embodiments, the amplification comprises use of a PCR primer that anneals to a sequence within an adapter and a PCR primer that anneals to a sequence within a probe annealed to the target region of the DNA.

In some embodiments, the methods herein comprise enriching for or capturing DNA comprising epigenetic and/or sequence-variable target regions. Such regions may be captured from an aliquot of a sample (e.g., a sample that has undergone attachment of adapters and amplification), while the step of partitioning the DNA with an agent that recognizes a modified cytosine, such as methyl cytosine, is performed on a separate aliquot of the sample. Enriching for or capturing DNA comprising epigenetic and/or sequence-variable target regions may comprise contacting the DNA with a first or second set of target-specific probes. Such target-specific probes may have any of the features described herein for sets of target-specific probes, including but not limited to in the embodiments set forth above and the sections relating to probes below. Capturing may be performed on one or more subsamples prepared during methods disclosed herein. In some embodiments, DNA is captured from the first subsample or the second subsample, e.g., the first subsample and the second subsample. In some embodiments, the subsamples are differentially tagged (e.g., as described herein) and then pooled before undergoing capture. Exemplary methods for capturing DNA comprising epigenetic and/or sequence-variable target regions can be found in, e.g., WO 2020/160414, which is hereby incorporated by reference.

The capturing step may be performed using conditions suitable for specific nucleic acid hybridization, which generally depend to some extent on features of the probes such as length, base composition, etc. Those skilled in the art will be familiar with appropriate conditions given general knowledge in the art regarding nucleic acid hybridization. In some embodiments, complexes of target-specific probes and DNA are formed.

In some embodiments, methods described herein comprise capturing a plurality of sets of target regions of cfDNA obtained from a subject. The target regions may comprise differences depending on whether they originated from a tumor or from healthy cells or from a certain cell type. The capturing step produces a captured set of cfDNA molecules. In some embodiments, cfDNA molecules corresponding to a sequence-variable target region set are captured at a greater capture yield in the captured set of cfDNA molecules than cfDNA molecules corresponding to an epigenetic target region set. In some embodiments, a method described herein comprises contacting cfDNA obtained from a subject with a set of target-specific probes, wherein the set of target-specific probes is configured to capture cfDNA corresponding to the sequence-variable target region set at a greater capture yield than cfDNA corresponding to the epigenetic target region set. For additional discussion of capturing steps, capture yields, and related aspects, see WO2020/160414, which is incorporated herein by reference for all purposes.

It can be beneficial to capture cfDNA corresponding to the sequence-variable target region set at a greater capture yield than cfDNA corresponding to the epigenetic target region set because a greater depth of sequencing may be necessary to analyze the sequence-variable target regions with sufficient confidence or accuracy than may be necessary to analyze the epigenetic target regions. The volume of data needed to determine fragmentation patterns (e.g., to test for perturbation of transcription start sites or CTCF binding sites) or fragment abundance (e.g., in hypermethylated and hypomethylated partitions) is generally less than the volume of data needed to determine the presence or absence of cancer-related sequence mutations. Capturing the target region sets at different yields can facilitate sequencing the target regions to different depths of sequencing in the same sequencing run (e.g., using a pooled mixture and/or in the same sequencing cell).

In some embodiments, the DNA is amplified. In some embodiments, amplification is performed before the capturing step. In some embodiments, amplification is performed after the capturing step. In some embodiments, amplification is performed before and after the capturing step. In various embodiments, the methods further comprise sequencing the captured DNA, e.g., to different degrees of sequencing depth for the epigenetic and sequence-variable target region sets, consistent with the discussion herein.

In some embodiments, a capturing step is performed with probes for a sequence-variable target region set and probes for an epigenetic target region set in the same vessel at the same time, e.g., the probes for the sequence-variable and epigenetic target region sets are in the same composition. This approach provides a relatively streamlined workflow. In some embodiments, the concentration of the probes for the sequence-variable target region set is greater that the concentration of the probes for the epigenetic target region set.

Alternatively, a capturing step is performed with a sequence-variable target region probe set in a first vessel and with an epigenetic target region probe set in a second vessel, or a contacting step is performed with a sequence-variable target region probe set at a first time and a first vessel and an epigenetic target region probe set at a second time before or after the first time. This approach allows for preparation of separate first and second compositions comprising captured DNA corresponding to a sequence-variable target region set and captured DNA corresponding to an epigenetic target region set. The compositions can be processed separately as desired (e.g., to partition based on methylation as described herein) and pooled in appropriate proportions to provide material for further processing and analysis such as sequencing.

In some embodiments, adapters are included in the DNA as described herein. In some embodiments, tags, which may be or include barcodes, are included in the DNA. In some embodiments, such tags are included in adapters. Tags can facilitate identification of the origin of a nucleic acid. For example, barcodes can be used to allow the origin (e.g., subject) whence the DNA came to be identified following pooling of a plurality of samples for parallel sequencing. This may be done concurrently with an amplification procedure, e.g., by providing the barcodes in a 5′ portion of a primer, e.g., as described herein. In some embodiments, adapters and tags/barcodes are provided by the same primer or primer set. For example, the barcode may be located 3′ of the adapter and 5′ of the target-hybridizing portion of the primer. Alternatively, barcodes can be added by other approaches, such as ligation, optionally together with adapters in the same ligation substrate.

Additional details regarding amplification, tags, and barcodes are discussed herein, which can be combined to the extent practicable with any of these embodiments.

E. Procedures that Affect a First Nucleobase in the DNA Differently from a Second Nucleobase in the DNA or Methylation-Sensitive Conversion Methods

In some embodiments, methods disclosed herein comprise a step of subjecting DNA, or a subsample thereof, to a procedure that affects a first nucleobase in the DNA differently from a second nucleobase in the DNA, wherein the first nucleobase is a modified or unmodified nucleobase, the second nucleobase is a modified or unmodified nucleobase different from the first nucleobase, and the first nucleobase and the second nucleobase have the same base pairing specificity. In some embodiments, the procedure chemically converts the first or second nucleobase such that the base pairing specificity of the converted nucleobase is altered. In some embodiments, DNA is subjected to a procedure that affects a first nucleobase in the DNA differently from a second nucleobase in the DNA before library preparation using the DNA, before a first amplification of the DNA, before dividing the DNA into a plurality of subsamples, or any combination thereof. In certain embodiments, the DNA is subjected to the procedure before or after contacting the DNA with a methylation-sensitive nuclease.

In some embodiments, the procedure that affects a first nucleobase of the DNA differently from a second nucleobase of the DNA is performed prior to the sequencing and/or (a) prior to or after the selectively depleting the target nucleic acid comprising the wild-type sequence, the target nucleic acid comprising the converted nucleotide, or the target nucleic acid that does not comprise the converted nucleotide; (b) prior to the amplifying the selectively digested population of target nucleic acids; (c) prior to or after the partitioning the population of target nucleic acids into a plurality of subsamples; and/or (d) prior to or after a step of enriching for one or more sets of target regions of DNA.

In some embodiments, if the first nucleobase is a modified or unmodified adenine, then the second nucleobase is a modified or unmodified adenine; if the first nucleobase is a modified or unmodified cytosine, then the second nucleobase is a modified or unmodified cytosine; if the first nucleobase is a modified or unmodified guanine, then the second nucleobase is a modified or unmodified guanine; and if the first nucleobase is a modified or unmodified thymine, then the second nucleobase is a modified or unmodified thymine (where modified and unmodified uracil are encompassed within modified thymine for the purpose of this step).

In some embodiments, the first nucleobase is a modified or unmodified cytosine, then the second nucleobase is a modified or unmodified cytosine. For example, first nucleobase may comprise unmodified cytosine (C) and the second nucleobase may comprise one or more of 5-methylcytosine (mC) and 5-hydroxymethylcytosine (hmC). Alternatively, the second nucleobase may comprise C and the first nucleobase may comprise one or more of mC and hmC. Other combinations are also possible, such as where one of the first and second nucleobases comprises mC and the other comprises hmC.

In some embodiments, the procedure that affects a first nucleobase in the DNA differently from a second nucleobase in the DNA comprises bisulfite conversion. Treatment with bisulfite converts unmodified cytosine and certain modified cytosine nucleotides (e.g. 5-formyl cytosine (fC) or 5-carboxylcytosine (caC)) to uracil whereas other modified cytosines (e.g., 5-methylcytosine, 5-hydroxylmethylcystosine) are not converted. Thus, where bisulfite conversion is used, the first nucleobase comprises one or more of unmodified cytosine, 5-formyl cytosine, 5-carboxylcytosine, or other cytosine forms affected by bisulfite, and the second nucleobase may comprise one or more of mC and hmC, such as mC and optionally hmC. Sequencing of bisulfite-treated DNA identifies positions that are read as cytosine as being mC or hmC positions. Meanwhile, positions that are read as T are identified as being T or a bisulfite-susceptible form of C, such as unmodified cytosine, 5-formyl cytosine, or 5-carboxylcytosine. Performing bisulfite conversion, such as on a DNA sample as described herein, facilitates identifying positions containing mC or hmC using the sequence reads obtained from the exemplary sample. For an exemplary description of bisulfite conversion, see, e.g., Moss et al., Nat Commun. 2018; 9:5068.

In some embodiments, the procedure that affects a first nucleobase in the DNA differently from a second nucleobase in the DNA comprises oxidative bisulfite (Ox-BS) conversion. This procedure first converts hmC to fC, which is bisulfite susceptible, followed by bisulfite conversion. Thus, when oxidative bisulfite conversion is used, the first nucleobase comprises one or more of unmodified cytosine, fC, caC, hmC, or other cytosine forms affected by bisulfite, and the second nucleobase comprises mC. Sequencing of Ox-BS converted DNA identifies positions that are read as cytosine as being mC positions. Meanwhile, positions that are read as T are identified as being T, hmC, or a bisulfite-susceptible form of C, such as unmodified cytosine, fC, or hmC. Performing Ox-BS conversion, such as on a DNA sample as described herein, thus facilitates identifying positions containing mC using the sequence reads obtained from the sample. For an exemplary description of oxidative bisulfite conversion, see, e.g., Booth et al., Science 2012; 336:934-937.

In some embodiments, the procedure that affects a first nucleobase in the DNA differently from a second nucleobase in the DNA comprises Tet-assisted bisulfite (TAB) conversion. In TAB conversion, hmC is protected from conversion and mC is oxidized in advance of bisulfite treatment, so that positions originally occupied by mC are converted to U while positions originally occupied by hmC remain as a protected form of cytosine. For example, as described in Yu et al., Cell 2012; 149:1368-80, β-glucosyl transferase can be used to protect hmC (forming 5-glucosylhydroxymethylcytosine (ghmC)), then a TET protein such as mTet1 can be used to convert mC to caC, and then bisulfite treatment can be used to convert C and caC to U while ghmC remains unaffected.

Alternatively, a carbamoyltransferase enzyme, such as 5-hydroxymethylcytosine carbamoyltransferase as described in Yang et al., Bio-protocol, 2023; 12(17): e4496, can be used to protect hmC (by converting hmC to 5-carbamoyloxymethylcytosine (5cmC)), then a TET protein such as mTet1 or a TET2 comprising a T1372S mutation, can be used to convert mC to caC, and then bisulfite treatment can be used to convert C and caC to U while 5cmC remains unaffected. Thus, when TAB conversion is used, the first nucleobase comprises one or more of unmodified cytosine, fC, caC, mC, or other cytosine forms affected by bisulfite, and the second nucleobase comprises hmC. Sequencing of TAB-converted DNA identifies positions that are read as cytosine as being hmC positions. Meanwhile, positions that are read as T are identified as being T, mC, or a bisulfite-susceptible form of C, such as unmodified cytosine, fC, or caC. Performing TAB conversion, such as on a DNA sample as described herein, thus facilitates identifying positions containing hmC using the sequence reads obtained from the sample.

In some embodiments, the procedure that affects a first nucleobase in the DNA differently from a second nucleobase in the DNA comprises Tet-assisted conversion with a substituted borane reducing agent, optionally wherein the substituted borane reducing agent is 2-picoline borane, borane pyridine, tert-butylamine borane, or ammonia borane. In Tet-assisted pic-borane conversion with a substituted borane reducing agent conversion, a TET protein is used to convert mC and hmC to caC, without affecting unmodified C. caC, and fC if present, are then converted to dihydrouracil (DHU) by treatment with 2-picoline borane (pic-borane) or another substituted borane reducing agent such as borane pyridine, tert-butylamine borane, or ammonia borane, also without affecting unmodified C. See, e.g., Liu et al., Nature Biotechnology 2019; 37:424-429 (e.g., at Supplementary FIG. 1 and Supplementary Note 7). Thus, when this type of conversion is used, the first nucleobase comprises one or more of 5mC, 5fC, 5caC, or 5hmC, and the second nucleobase comprises unmodified cytosine. DHU is read as a T in sequencing. Thus, when this type of conversion is used, the first nucleobase comprises one or more of mC, fC, caC, or hmC, and the second nucleobase comprises unmodified cytosine. Sequencing of the converted DNA identifies positions that are read as cytosine as being unmodified C positions. Meanwhile, positions that are read as T are identified as being T, mC, fC, caC, or hmC. Performing TAP conversion, such as on a DNA sample as described herein, thus facilitates identifying positions containing unmodified C using the sequence reads obtained from the sample. This procedure encompasses Tet-assisted pyridine borane sequencing (TAPS), described in further detail in Liu et al. 2019, supra.

Alternatively, protection of hmC (e.g., using βGT or 5-hydroxymethylcytosine carbamoyltransferase) can be combined with Tet-assisted conversion with a substituted borane reducing agent, e.g. as described above. In this method (TAPS-β), 5hmC can be protected from conversion, for example through glucosylation using β-glucosyl transferase (βGT), forming 5-glucosylhydroxymethylcytosine (5ghmC), or through carbamoylation using 5-hydroxymethylcytosine carbamoyltransferase, forming 5cmC. This is described in Yu et al., Cell 2012; 149: 1368-80. Treatment with a TET protein, such as mTet1 or a TET2 comprising a T1372S mutation, then converts mC to caC but does not convert C, 5ghmC, or 5cmC. 5caC is then converted to DHU by treatment with pic-borane or another substituted borane reducing agent such as borane pyridine, tert-butylamine borane, or ammonia borane, also without affecting ghmC, 5cmC, or unmodified C. Thus, when Tet-assisted conversion with a substituted borane reducing agent is used, the first nucleobase comprises mC, and the second nucleobase comprises one or more of unmodified cytosine or hmC, such as unmodified cytosine and optionally hmC, fC, and/or caC. Sequencing of the converted DNA identifies positions that are read as cytosine as being either hmC or unmodified C positions. Meanwhile, positions that are read as T are identified as being T, fC, caC, or mC. Performing TAPSβ conversion, such as on a DNA sample as described herein, thus facilitates distinguishing positions containing unmodified C or hmC on the one hand from positions containing mC using the sequence reads obtained from the sample. For an exemplary description of this type of conversion, see, e.g., Liu et al., Nature Biotechnology 2019; 37:424-429. 5-hydroxymethylcytosine carbamoyltransferase is described in Yang et al., Bio-protocol, 2023; 12(17): e4496.

In some embodiments, the procedure that affects a first nucleobase in the DNA differently from a second nucleobase in the DNA comprises chemical-assisted conversion with a substituted borane reducing agent, optionally wherein the substituted borane reducing agent is 2-picoline borane, borane pyridine, tert-butylamine borane, or ammonia borane. In chemical-assisted conversion with a substituted borane reducing agent, an oxidizing agent such as potassium perruthenate (KRuO4) (also suitable for use in ox-BS conversion) is used to specifically oxidize hmC to fC. Treatment with pic-borane or another substituted borane reducing agent such as borane pyridine, tert-butylamine borane, or ammonia borane converts fC and caC to DHU but does not affect mC or unmodified C. Thus, when this type of conversion is used, the first nucleobase comprises one or more of hmC, fC, and caC, and the second nucleobase comprises one or more of unmodified cytosine or mC, such as unmodified cytosine and optionally mC. Sequencing of the converted DNA identifies positions that are read as cytosine as being either mC or unmodified C positions. Meanwhile, positions that are read as T are identified as being T, fC, caC, or hmC. Performing this type of conversion, such as on a DNA sample as described herein, thus facilitates distinguishing positions containing unmodified C or mC on the one hand from positions containing hmC using the sequence reads obtained from the sample. For an exemplary description of this type of conversion, see, e.g., Liu et al., Nature Biotechnology 2019; 37:424-429.

In some embodiments, the procedure that affects a first nucleobase in the DNA differently from a second nucleobase in the DNA comprises APOBEC-coupled epigenetic (ACE) conversion. In ACE conversion, an AID/APOBEC family DNA deaminase enzyme such as APOBEC3A (A3A) is used to deaminate unmodified cytosine and mC without deaminating hmC, fC, or caC. Thus, when ACE conversion is used, the first nucleobase comprises unmodified C and/or mC (e.g., unmodified C and optionally mC), and the second nucleobase comprises hmC. Sequencing of ACE-converted DNA identifies positions that are read as cytosine as being hmC, fC, or caC positions. Meanwhile, positions that are read as T are identified as being T, unmodified C, or mC. Performing ACE conversion on a DNA sample as described herein thus facilitates distinguishing positions containing hmC from positions containing mC or unmodified C using the sequence reads obtained from the sample. For an exemplary description of ACE conversion, see, e.g., Schutsky et al., Nature Biotechnology 2018; 36: 1083-1090.

In some embodiments, the procedure that affects a first nucleobase in the DNA differently from a second nucleobase in the DNA comprises enzymatic conversion of the first nucleobase, e.g., as in EM-Seq. See, e.g., Vaisvila R, et al. (2019) EM-seq: Detection of DNA methylation at single base resolution from picograms of DNA. bioRxiv; DOI: 10.1101/2019.12.20.884692, available at www.biorxiv.org/content/10.1101/2019.12.20.884692v1. For example, TET2 and T4-βGT or 5-hydroxymethylcytosine carbamoyltransferase (described in Yang et al., Bio-protocol, 2023; 12(17): e4496) can be used to convert 5mC and 5hmC into substrates that cannot be deaminated by a deaminase (e.g., APOBEC3A), and then a deaminase (e.g., APOBEC3A) can be used to deaminate unmodified cytosines converting them to uracils.

In some embodiments, the procedure that affects a first nucleobase in the DNA differently from a second nucleobase in the DNA comprises enzymatic conversion of the first nucleobase using a non-specific, modification-sensitive double-stranded DNA deaminase, e.g., as in SEM-seq. See, e.g., Vaisvila et al. (2023) Discovery of novel DNA cytosine deaminase activities enables a nondestructive single-enzyme methylation sequencing method for base resolution high-coverage methylome mapping of cell-free and ultra-low input DNA. bioRxiv; DOI: 10.1101/2023.06.29.547047, available at https://www.biorxiv.org/content/10.1101/2023.06.29.547047v1. SEM-Seq employs a non-specific, modification-sensitive double-stranded DNA deaminase (MsddA) in a nondestructive single-enzyme 5-methylctyosine sequencing (SEM-seq) method that deaminates unmodified cytosines. Accordingly, SEM-seq does not require the TET2 and T4-βGT or 5-hydroxymethylcytosine carbamoyltransferase protection and denaturing steps that are of use, e.g., in APOEC3A-based protocols. Additionally, MsddA does not deaminate 5-formylated cytosines (5fC) or 5-carboxylated cytosines (5caC). In SEM-seq, unmodified cytosines in the DNA are deaminated to uracil and is read as “T” during sequencing. Modified cytosines (e.g., 5mC) are not converted and are read as “C” during sequencing. Cytosines that are read as thymines are identified as unmodified (e.g., unmethylated) cytosines or as thymines in the DNA. Performing SEM-seq conversion thus facilitates identifying positions containing 5mC using the sequence reads obtained. In some embodiments, the procedure that affects a first nucleobase in the DNA differently from a second nucleobase in the DNA comprises enzymatic conversion of the first nucleobase using MsddA.

In some embodiments, the procedure that affects a first nucleobase in the DNA differently from a second nucleobase in the DNA of the first subsample converts a modified nucleoside. In some embodiments, the conversion procedure which converts a modified nucleosides comprises enzymatic conversion, such as DM-seq, for example, as described in WO2023/288222A1. In DM-seq, unmodified cytosines in the DNA are enzymatically protected from a subsequent deamination step wherein 5mC in 5mCpG is converted to T. The enzymatically protected unmodified (e.g., unmethylated) cytosines are not converted and are read as “C” during sequencing. Cytosines that are read as thymines (in a CpG context) are identified as methylated cytosines in the DNA. Thus, when this type of conversion is used, the first nucleobase comprises unmodified (such as unmethylated) cytosine, and the second nucleobase comprises modified (such as methylated) cytosine. Sequencing of the converted DNA identifies positions that are read as cytosine as being unmodified C positions. Meanwhile, positions that are read as T are identified as being T or 5mC. Performing DM-seq conversion thus facilitates identifying positions containing 5mC using the sequence reads obtained.

Exemplary cytosine deaminases for use herein include APOBEC enzymes, for example, APOBEC3A. Generally, AID/APOBEC family DNA deaminase enzymes such as APOBEC3A (A3A) are used to deaminate (unprotected) unmodified cytosine and 5mC. For an exemplary description of APOBEC conversion, see, e.g., Schutsky et al., Nature Biotechnology 2018; 36: 1083-1090.

The enzymatic protection of unmodified cytosines in the DNA comprises addition of a protective group to the unmodified cytosines. Such protective groups can comprise an alkyl group, an alkyne group, a carboxyl group, a carboxyalkyl group, an amino group, a hydroxymethyl group, a glucosyl group, a glucosylhydroxymethyl group, an isopropyl group, or a dye. For example, DNA can be treated with a methyltransferase, such as a CpG-specific methyltransferase, which adds the protective group to unmodified cytosines. The term methyltransferase is used broadly herein to refer to enzymes capable of transferring a methyl or substituted methyl (e.g., carboxymethyl) to a substrate (e.g., a cytosine in a nucleic acid). In some embodiments, the DNA is contacted with a CpG-specific DNA methyltransferase (MTase), such as a CpG-specific carboxymethyltransferase (CxMTase), and a substituted methyl donor, such as a carboxymethyl donor (e.g., carboxymethyl-S-adenosyl-L-methionine). See, e.g., WO2021/236778A2. In particular embodiments, the CxMTase can facilitate the addition of a protective carboxymethyl group to an unmethylated cytosine. In some embodiments, the unmethylated cytosine is unmodified cytosine. The carboxymethyl group can prevent deamination of the cytosine during a deamination step (such as a deamination step using an APOBEC enzyme, such as A3A). Substituted methyl or carboxymethyl donors useful in the disclosed methods include but are not limited to, S-adenosyl-L-methionine (SAM) analogs, optionally wherein the SAM analog is carboxy-S-adenosyl-L-methionine (CxSAM). SAM analogs are described, for example, in WO2022/197593A1. The MTase may be, for example, a CpG methyltransferase from Spiroplasma sp. strain MQ1 (M.Sssl), DNA-methyltransferase 1 (DNMT1), DNA-methyltransferase 3 alpha (DNMT3A), DNA-methyltransferase 3 beta (DNMT3B), or DNA adenine methyltransferase (Dam). The CxMTase may be a CpG methyltransferase from Mycoplasma penetrans (M.Mpel). In a particular embodiment, the methyltransferase enzyme is a variant of M.Mpel, wherein the amino acid corresponding to position 374 is R or K.

In one embodiment, the methyltransferase enzyme is a variant of M.Mpel having an N374R substitution or an N374K substitution. The methyltransferase variant can further comprise one or more amino acid substitutions selected from a) substitution of one or both residues T300 and E305 with S, A, G, Q, D, or N; b) substitution of one or more residues A323, N306, and Y299 with a positively charged amino acid selected from K, R or H; and/or c) substitution of S323 with A, G, K, R or H, which may enhance the activity of the enzyme.

Optionally, the conversion procedure further includes enzymatic protection of 5hmCs, such as by glucosylation of the 5hmCs (e.g., using βGT) or by carbamoylation of the 5hmCs (e.g., using 5-hydroxymethylcytosine carbamoyltransferase), in the DNA prior to the deamination of unprotected modified cytosines. In this method, 5hmC can be protected from conversion, for example through glucosylation using β-glucosyl transferase (βGT), forming (5-glucosylhydroxymethylcytosine) 5ghmC, or through carbamoylation using 5-hydroxymethylcytosine carbamoyltransferase, forming 5cmC. This is described, for example, in Yu et al., Cell 2012; 149: 1368-80, and in Yang et al., Bio-protocol, 2023; 12(17): e4496. Glucosylation or carbamoylation of 5hmC can reduce or eliminate deamination of 5hmC by a deaminase such as APOBEC3A. Treatment with an MTase or CxMTase then adds a protecting group to unmodified (unmethylated) cytosines in the DNA. 5mC (but not protected, unmodified cytosine and not 5ghmC or 5cmC) is then deaminated (converted to T in the case of 5mC) by treatment with a deaminase, for example, an APOBEC enzyme (such as APOBEC3A). Sequencing of the converted DNA identifies positions that are read as cytosine as being either 5hmC or unmodified C positions. Meanwhile, positions that are read as T are identified as being T or 5mC. Performing DM-seq conversion with glucosylation of 5hmC on a sample as described herein thus facilitates distinguishing positions containing unmodified C or 5hmC on the one hand from positions containing 5mC using the sequence reads obtained.

Also provided herein are methods in which alternative base conversion schemes are used. For example, unmethylated cytosines can be left intact while methylated cytosines and hydroxymethylcytosines are converted to a base read as a thymine (e.g., uracil, thymine, or dihydrouracil).

In some embodiments, methylating a cytosine in at least one first complementary strand or second complementary strand comprises contacting the cytosine with a methyltransferase such as DNMT1 or DNMT5. In such embodiments, the step of oxidizing a 5-hydroxymethylated cytosine to 5-formylcytosine (such as by contacting the 5-hydroxymethyl cytosine in a first strand and a second strand with KRuO4) can be optional.

In some embodiments, converting the modified cytosine in at least one first or second strand to a thymine or a base read as thymine comprises oxidizing a hydroxymethyl cytosine, e.g., the hydroxymethyl cytosine is oxidized to formylcytosine. In some embodiments, oxidizing the hydroxymethyl cytosine to formylcytosine comprises contacting the hydroxymethyl cytosine with a ruthenate, such as potassium ruthenate (KRuO4).

In some embodiments, the modified cytosine is converted to thymine, uracil, or dihydrouracil. In any such embodiments, amplification methods may comprise uracil- and/or dihydrouracil-tolerant amplification methods, such as PCR using a uracil- and/or dihydrouracil-tolerant DNA polymerase.

In some embodiments, the method comprises converting a formylcytosine and/or a methylcytosine to carboxylcytosine as part of converting the modified cytosine in at least one first or second strand to a thymine or a base read as thymine. For example, converting the formylcytosine and/or the methylcytosine to carboxylcytosine can comprise contacting the formylcytosine and/or the methylcytosine with a TET enzyme, such as TET1, TET2, TET3, or a TET2 comprising a T1372S mutation. In some embodiments, the method comprises reducing the carboxylcytosine as part of converting the modified cytosine in at least one first or second strand to a thymine or a base read as thymine, and/or the carboxylcytosine is reduced to dihydrouracil. In some embodiments, reducing the carboxylcytosine comprises contacting the carboxylcytosine with a borane or borohydride reducing agent.

In some embodiments, the borane or borohydride reducing agent comprises pyridine borane, 2-picoline borane, borane, tert-butylamine borane, ammonia borane, sodium borohydride, sodium cyanoborohydride (NaBH3CN), lithium borohydride (LiBH4), ethylenediamine borane, dimethylamine borane, sodium triacetoxyborohydride, morpholine borane, 4-methylmorpholine borane, trimethylamine borane, dicyclohexylamine borane, or a salt thereof. In other embodiments, the reducing agent comprises lithium aluminum hydride, sodium amalgam, amalgam, sulfur dioxide, dithionate, thiosulfate, iodide, hydrogen peroxide, hydrazine, diisobutylaluminum hydride, oxalic acid, carbon monoxide, cyanide, ascorbic acid, formic acid, dithiothreitol, beta-mercaptoethanol, or any combination thereof.

Various TET enzymes may be used in the disclosed methods as appropriate. In some embodiments, the one or more TET enzymes comprise TETv. TETv is described in U.S. Pat. No. 10,260,088. In some embodiments, the one or more TET enzymes comprise TETcd. TETcd is described in U.S. Pat. No. 10,260,088. In some embodiments, the one or more TET enzymes comprise TET1. In some embodiments, the one or more TET enzymes comprise TET2. TET2 may be expressed and used as a fragment comprising TET2 residues 1129-1480 joined to TET2 residues 1844-1936 by a linker as described, e.g., in U.S. Pat. No. 10,961,525. In some embodiments, the one or more TET enzymes comprise TET1 and TET2. In some embodiments, the one or more TET enzymes comprise a V1900 TET mutant, such as a V1900A, V1900C, V1900G, V1900I, or V1900P TET mutant. In some embodiments, the one or more TET enzymes comprise a V1900 TET2 mutant, such as a V1900A, V1900C, V1900G, V1900I, or V1900P TET2 mutant. It can be beneficial to use a TET enzyme that maximizes formation of 5-carboxylcytosine (5-caC) relative to less oxidized modified cytosines, particularly 5-formylcytosine, because 5-caC is not a substrate for enzymatic deamination, e.g., by APOBEC enzymes such as APOBEC3A. Maximizing formation of 5-caC thus reduces the risk of false calls in which a base is identified as unmethylated because it underwent deamination even though it was methylated (or hydroxymethylated) in the original sample. Accordingly, in some embodiments, the TET enzyme comprises a mutation that increases formation of 5-caC. In some embodiments, the one or more TET enzymes comprise a TET2 enzyme comprising a T1372S mutation, such as TET2-CS-T1372S and TET2-CD-T1372S. A TET2 comprising a T1372S mutation is described in U.S. Pat. No. 10,961,525 and may be expressed and used as a fragment comprising TET2 residues 1129-1480 joined to TET2 residues 1844-1936 by a linker. Position 1372 of TET2 corresponds to position 258 of SEQ ID NO: 21 (wild type TET2 catalytic domain) of U.S. Pat. No. 10,961,525. Thus, the sequence of a T1372S TET2 catalytic domain may be obtained by changing the threonine at position 258 of SEQ ID NO: 21 of U.S. Pat. No. 10,961,525 to serine. TET2 comprising a T1372S mutation is also described in Liu et al., Nat Chem Biol. 2017 February; 13(2): 181-187. As demonstrated in Liu et al., TET2 comprising a T1372S mutation can more efficiently oxidize 5mC to produce 5-carboxylcytosine (5caC) than other versions of TET2 such as TET2 lacking a T1372S mutation. In some embodiments, the TET2 enzyme is a human TET2 enzyme comprising a T1372S mutation. Exemplary mutations are set forth above. “A mutation that increases formation of 5-caC” means that the TET enzyme having the mutation produces more 5-caC than a TET enzyme that lacks the mutation but is otherwise identical. 5-caC production can be measured as described, e.g., in Liu et al., Nat Chem Biol 13:181-187 (2017) (see Online Methods section, TET reactions in vitro subsection, “driving” conditions). Any variants and/or mutants described in Liu et al. (2017) can be used in the disclosed methods as appropriate.

Provided herein is a method comprising contacting DNA contacting DNA with a mutant TET2 enzyme (e.g. comprising a V1900A, V1900C, V1900G, V1900I, V1900P, or T1372S mutation) to oxidize 5-methylcytosine (5mC) and/or 5-hydroxymethylcytosine (5hmC) present in the DNA to 5-carboxycytosine (5caC), subsequently contacting at least a portion of the DNA with a substituted borane reducing agent, thereby converting 5-caC in the DNA to dihydrouracil (DHU), thereby producing treated DNA, and sequencing at least a portion of the treated DNA.

In some embodiments, the procedure that affects a first nucleobase in the DNA differently from a second nucleobase in the DNA comprises separating DNA originally comprising the first nucleobase from DNA not originally comprising the first nucleobase. In some such embodiments, the first nucleobase is hmC. DNA originally comprising the first nucleobase may be separated from other DNA using a labeling procedure comprising biotinylating positions that originally comprised the first nucleobase. In some embodiments, the first nucleobase is first derivatized with an azide-containing moiety, such as a glucosyl-azide containing moiety. The azide-containing moiety then may serve as a reagent for attaching biotin, e.g., through Huisgen cycloaddition chemistry. Then, the DNA originally comprising the first nucleobase, now biotinylated, can be separated from DNA not originally comprising the first nucleobase using a biotin-binding agent, such as avidin, neutravidin (deglycosylated avidin with an isoelectric point of about 6.3), or streptavidin. An example of a procedure for separating DNA originally comprising the first nucleobase from DNA not originally comprising the first nucleobase is hmC-seal, which labels hmC to form β-6-azide-glucosyl-5-hydroxymethylcytosine and then attaches a biotin moiety through Huisgen cycloaddition, followed by separation of the biotinylated DNA from other DNA using a biotin-binding agent. For an exemplary description of hmC-seal, see, e.g., Han et al., Mol. Cell 2016, 63: 711-719. This approach is useful for identifying fragments that include one or more hmC nucleobases.

In some embodiments, following such a separation, the method further comprises differentially tagging each of the DNA originally comprising the first nucleobase, the DNA not originally comprising the first nucleobase. The method may further comprise pooling the DNA originally comprising the first nucleobase and the DNA not originally comprising the first nucleobase following differential tagging. The DNA originally comprising the first nucleobase and the DNA not originally comprising the first nucleobase may then be used in downstream analyses. For example, the pooled DNA originally comprising the first nucleobase and the DNA not originally comprising the first nucleobase may be sequenced in the same sequencing cell (such as after being subjected to further treatments, such as those described herein) while retaining the ability to resolve whether a given read came from a molecule of DNA originally comprising the first nucleobase or DNA not originally comprising the first nucleobase using the differential tags.

In some embodiments, the first nucleobase is a modified or unmodified adenine, and the second nucleobase is a modified or unmodified adenine. In some embodiments, the modified adenine is N6-methyladenine (mA). In some embodiments, the modified adenine is one or more of N⁶-methyladenine (mA), N⁶-hydroxymethyladenine (hmA), or N⁶-formyladenine (fA).

Techniques comprising partitioning based on methylation status or methylated DNA immunoprecipitation (MeDIP) can be used to separate DNA containing modified bases such as mC, mA, caC (which may be generated by oxidation of mC or hmC with Tet2, e.g., before enzymatic conversion of unmodified C to U, e.g., using a deaminase such as APOBEC3A), or dihydrouracil from other DNA. See, e.g., Kumar et al., Frontiers Genet. 2018; 9: 640; Greer et al., Cell 2015; 161: 868-878. An antibody specific for mA is described in Sun et al., Bioessays 2015; 37:1155-62. Antibodies for various modified nucleobases, such as mC, caC, and forms of thymine/uracil including dihydrouracil or halogenated forms such as 5-bromouracil, are commercially available. Various modified bases can also be detected based on alterations in their base pairing specificity. For example, hypoxanthine is a modified form of adenine that can result from deamination and is read in sequencing as a G. See, e.g., U.S. Pat. No. 8,486,630; Brown, Genomes, 2nd Ed., John Wiley & Sons, Inc., New York, N.Y., 2002, chapter 14, “Mutation, Repair, and Recombination.”

### F. Captured Set; Target Regions

In some embodiments, nucleic acids captured or enriched using a method described herein comprise captured DNA, such as one or more captured sets of DNA. In some embodiments, the captured DNA comprise target regions that are differentially methylated in different immune cell types. In some embodiments, the immune cell types comprise rare or closely related immune cell types, such as activated and naive lymphocytes or myeloid cells at different stages of differentiation.

In some embodiments, a captured epigenetic target region set captured from a sample or first subsample comprises hypermethylation target regions. In some embodiments, the hypermethylation target regions are differentially or exclusively hypermethylated in one cell type or in one immune cell type, or in one immune cell type within a cluster. In some embodiments, the hypermethylation target regions are hypermethylated to an extent that is distinguishably higher or exclusively present in one cell type or one immune cell type or one immune cell type within a cluster. Such hypermethylation target regions may be hypermethylated in other cell types but not to the extent observed in the one cell type. In some embodiments, the hypermethylation target regions show lower methylation in healthy cfDNA than in at least one other tissue type.

In some embodiments, a captured epigenetic target region set captured from a sample or second subsample comprises hypomethylation target regions. In some embodiments, the hypomethylation target regions are exclusively hypomethylated in one cell type or in one immune cell type or in one immune cell type within a cluster. In some embodiments, the hypomethylation target regions are hypomethylated to an extent that is exclusively present in one cell type or one immune cell type or in one immune cell type within a cluster.

Such hypomethylation target regions may be hypomethylated in other cell types but not to the extent observed in the one cell type. In some embodiments, the hypomethylation target regions show higher methylation in healthy cfDNA than in at least one other tissue type. [0248] Without wishing to be bound by any particular theory, in an individual with cancer, proliferating or activated immune cells (and potentially also cancer cells) may shed more DNA into the bloodstream than immune cells in a healthy individual (and healthy cells of the same tissue type, respectively). As such, the distribution of cell type and/or tissue of origin of cfDNA may change upon carcinogenesis. For example, the distribution of immune cell type of origin may change in a subject having cancer, precancer, infection, transplant rejection, or other disease or disorder directly or indirectly affecting the immune system. The status of epigenetic target regions of certain immune cell types likewise may change in a subject having such a disease relative to a healthy subject or relative to the same subject prior to having the disease or disorder. Thus, variations in hypermethylation and/or hypomethylation can be an indicator of disease. For example, an increase in the level of hypermethylation target regions and/or hypomethylation target regions in a subsample following a partitioning step can be an indicator of the presence (or recurrence, depending on the history of the subject) of cancer.

Exemplary hypermethylation target regions and hypomethylation target regions useful for distinguishing between various cell types, including but not limited to immune cell types, have been identified by analyzing DNA obtained from various cell types via whole genome bisulfite sequencing, as described, e.g., in Stunnenberg, H. G. et. al., “The International Human Epigenome Consortium: A Blueprint for Scientific Collaboration and Discovery,” Cell 167, 1145 (2016) (doi.org/10.1186/sl3059-020-02065-5). Whole-genome bisulfite sequencing data is available from the Blueprint consortium, available on the internet at dcc.blueprint-epigenome.eu.

In some embodiments, first and second captured target region sets comprise, respectively, DNA corresponding to a sequence-variable target region set and DNA corresponding to an epigenetic target region set, for example, as described in WO 2020/160414. The first and second captured sets may be combined to provide a combined captured set.

Where DNA (e.g., a sample or subsample) has been subjected to a procedure such as bisulfite conversion, treatment with a deaminase, or any of the other such procedures mentioned herein that alter the base-pairing specificity of certain bases, enrichment or capture may use oligonucleotides (e.g., primers or probes) specific for the altered or unaltered sequence, as desired.

In some embodiments in which a captured set comprising DNA corresponding to the sequence-variable target region set and the epigenetic target region set includes a combined captured set as discussed above, the DNA corresponding to the sequence-variable target region set may be present at a greater concentration than the DNA corresponding to the epigenetic target region set, e.g., a 1.1 to 1.2-fold greater concentration, a 1.2- to 1.4-fold greater concentration, a 1.4- to 1.6-fold greater concentration, a 1.6- to 1.8-fold greater concentration, a 1.8- to 2.0-fold greater concentration, a 2.0- to 2.2-fold greater concentration, a 2.2- to 2.4-fold greater concentration a 2.4- to 2.6-fold greater concentration, a 2.6- to 2.8-fold greater concentration, a 2.8- to 3.0-fold greater concentration, a 3.0- to 3.5-fold greater concentration, a 3.5- to 4.0, a 4.0- to 4.5-fold greater concentration, a 4.5- to 5.0-fold greater concentration, a 5.0- to 5.5-fold greater concentration, a 5.5- to 6.0-fold greater concentration, a 6.0- to 6.5-fold greater concentration, a 6.5- to 7.0-fold greater, a 7.0- to 7.5-fold greater concentration, a 7.5- to 8.0-fold greater concentration, an 8.0- to 8.5-fold greater concentration, an 8.5- to 9.0-fold greater concentration, a 9.0- to 9.5-fold greater concentration, 9.5- to 10.0-fold greater concentration, a 10- to 11-fold greater concentration, an 11- to 12-fold greater concentration a 12- to 13-fold greater concentration, a 13- to 14-fold greater concentration, a 14- to 15-fold greater concentration, a 15- to 16-fold greater concentration, a 16- to 17-fold greater concentration, a 17- to 18-fold greater concentration, an 18- to 19-fold greater concentration, a 19- to 20-fold greater concentration, a 20- to 30-fold greater concentration, a 30- to 40-fold greater concentration, a 40- to 50-fold greater concentration, a 50- to 60-fold greater concentration, a 60- to 70-fold greater concentration, a 70- to 80-fold greater concentration, a 80- to 90-fold greater concentration, or a 90- to 100-fold greater concentration. The degree of difference in concentrations accounts for normalization for the footprint sizes of the target regions, as discussed in the definition section.

### 1. Epigenetic Target Region Set

In some embodiments, an epigenetic target region set may comprise one or more types of target regions likely to differentiate DNA from different immune cell types and other non-immune cell types and/or to differentiate neoplastic (e.g., tumor or cancer) cells and from healthy cells, e.g., non-neoplastic circulating cells. Exemplary types of such regions are discussed in detail herein. The epigenetic target region set may also comprise one or more control regions, e.g., as described herein.

In some embodiments, the epigenetic target region set has a footprint of at least 100 kb, e.g., at least 200 kb, at least 300 kb, or at least 400 kb. In some embodiments, the epigenetic target region set has a footprint in the range of 100-1000 kb, e.g., 100-200 kb, 200-300 kb, 300-400 kb, 400-500 kb, 500-600 kb, 600-700 kb, 700-800 kb, 800-900 kb, and 900-1,000 kb.

a. Hypermethylation Target Regions

In some embodiments, the epigenetic target region set comprises one or more hypermethylation target regions. In some embodiments, hypermethylation target regions are exclusively hypermethylated in one immune cell type or hypermethylated to a greater extent in one immune cell type than in any other immune cell type or than in any other immune cell type within the same immune cell cluster. In some such embodiments, hypermethylation target regions indicate the levels of particular immune cell types from which the DNA originated, including rare immune cell types such as activated B cells (including memory B cells and plasma cells), activated T cells (including regulatory T cells (Tregs), CD4 effector memory T cells, CD4 central memory T cells, CD8 effector memory T cells, and CD8 central memory T cells), and natural killer (NK) cells. Methylation patterns of hypermethylation target regions that are useful for deconvoluting immune cell types may further change in certain disease states, such as cancer. Thus, in some embodiments, hypermethylation target regions that are useful for deconvoluting immune cell types are also useful for determining the likelihood that the subject from which the sample was obtained has cancer or precancer. In some such embodiments, hypermethylation target regions are useful for determining whether levels of particular immune cell types are abnormal and whether such abnormal levels are likely related to the presence of cancer or precancer, or if they are related to a different disease or condition other than cancer or precancer.

In some embodiments, certain hypermethylation target regions exhibit an increase in the level of observed methylation, e.g., are hypermethylated, in DNA produced by neoplastic cells, such as tumor or cancer cells. Detection of such hypermethylation target regions, e.g., in conjunction with detection of hypermethylation target regions indicative of immune cell types, may further increase the specificity and/or sensitivity of methods described herein. In some embodiments, such increases in observed methylation in hypermethylated target regions indicate an increased likelihood that a sample (e.g., of cfDNA) was obtained from a subject having cancer. For example, hypermethylation of promoters of tumor suppressor genes has been observed repeatedly. See, e.g., Kang et ah, Genome Biol. 18:53 (2017) and references cited therein. In another example, as discussed above, hypermethylation target regions can include regions that do not necessarily differ in methylation in cancerous tissue relative to DNA from healthy tissue of the same type, but do differ in methylation (e.g., have more methylation) relative to cfDNA that is typical in healthy subjects. Where, for example, the presence of a cancer results in increased cell death such as apoptosis of cells of the tissue type corresponding to the cancer, such a cancer can be detected at least in part using such hypermethylation target regions. In some embodiments, hypermethylation target regions useful for determining the likelihood that a subject has cancer are different than the hypermethylation target regions useful for determining the levels of particular immune cell types. In some embodiments, at least some of the hypermethylation target regions useful for determining the likelihood that a subject has cancer are the same as the hypermethylation target regions useful for determining the levels of particular immune cell types.

An extensive discussion of methylation variable target regions in colorectal cancer is provided in Lam et al., Biochim Biophys Acta. 1866:106-20 (2016). These include VIM, SEPT9, ITGA4, OSM4, GATA4 and NDRG4. An exemplary set of hypermethylation target regions based on colorectal cancer (CRC) studies is provided in Table 1. Many of these genes likely have relevance to cancers beyond colorectal cancer, for example, TP53 is widely recognized as a critically important tumor suppressor and hypermethylation-based inactivation of this gene may be a common oncogenic mechanism.

TABLE 1
Exemplary Hypermethylation Target Regions based on CRC studies.

| | Additional | |
|---|---|---|
| Gene Name | Gene Name | Chromosome |
| VIM | | chr10 |
| SEPT9 | | chr17 |
| CYCD2 | CCND2 | chr12 |
| TFPI2 | | chr7 |
| GATA4 | | chr8 |
| RARB2 | RARB | chr3 |
| p16INK4a | CDKN2A | chr9 |
| MGMT | | chr10 |
| APC | | chr5 |
| NDRG4 | | chr16 |
| HLTF | | chr3 |
| HPP1 | TMEFF2 | chr2 |
| hMLH1 | MLH1 | chr3 |
| RASSF1A | RASSF1 | chr3 |
| CDH13 | | chr16 |
| IGFBP3 | | chr7 |
| ITGA4 | | chr2 |

In some embodiments, the hypermethylation target regions comprise a plurality of loci listed in Table 1, e.g., at least 10%, 20%, 30%, 40%, 50%, 60%, 70%, 80%, 90%, or 100% of the loci listed in Table 1. For example, for each locus included as a target region, there may be one or more probes with a hybridization site that binds between the transcription start site and the stop codon (the last stop codon for genes that are alternatively spliced) of the gene, or in the promoter region of the gene. In some embodiments, the one or more probes bind within 300 bp of the transcription start site of a gene in Table 1, e.g., within 200 or 100 bp.

Methylation variable target regions in various types of lung cancer are discussed in detail, e.g., in Ooki et al., Clin. Cancer Res. 23:7141-52 (2017); Belinksy, Annu. Rev. Physiol. 77:453-74 (2015); Hulbert et al., Clin. Cancer Res. 23:1998-2005 (2017); Shi et al., BMC Genomics 18:901 (2017); Schneider et al., BMC Cancer. 11:102 (2011); Lissa et al., Transl Lung Cancer Res 5(5):492-504 (2016); Skvortsova et al., Br. J. Cancer. 94(10): 1492-1495 (2006); Kim et al., Cancer Res. 61:3419-3424 (2001); Furonaka et al., Pathology International 55:303-309 (2005); Gomes et al., Rev. Port. Pneumol. 20:20-30 (2014); Kim et al., Oncogene. 20:1765-70 (2001); Hopkins-Donaldson et al., Cell Death Differ. 10:356-64 (2003); Kikuchi et al., Clin. Cancer Res. 11:2954-61 (2005); Heller et al., Oncogene 25:959-968 (2006); Licchesi et al., Carcinogenesis. 29:895-904 (2008); Guo et al., Clin. Cancer Res. 10:7917-24 (2004); Palmisano et al., Cancer Res. 63:4620-4625 (2003); and Toyooka et al., Cancer Res. 61:4556-4560, (2001).

An exemplary set of hypermethylation target regions based on lung cancer studies is provided in Table 2. Many of these genes likely have relevance to cancers beyond lung cancer; for example, Casp8 (Caspase 8) is a key enzyme in programmed cell death and hypermethylation-based inactivation of this gene may be a common oncogenic mechanism not limited to lung cancer. Additionally, a number of genes appear in both Tables 1 and 2, indicating generality.

TABLE 2
Exemplary Hypermethylation Target
Regions based on Lung Cancer studies

| Gene Name | Chromosome |
|---|---|
| MARCH11 | chr5 |
| TAC1 | chr7 |
| TCF21 | chr6 |
| SHOX2 | chr3 |
| p16 | chr3 |
| Casp8 | chr2 |
| CDH13 | chr16 |
| MGMT | chr10 |
| MLH1 | chr3 |
| MSH2 | chr2 |
| TSLC1 | chr11 |
| APC | chr5 |
| DKK1 | chr10 |
| DKK3 | chr11 |
| LKB1 | chr11 |
| WIF1 | chr12 |
| RUNX3 | chr1 |
| GATA4 | chr8 |
| GATA5 | chr20 |
| PAX5 | chr9 |
| E-Cadherin | chr16 |
| H-Cadherin | chr16 |

Any of the foregoing embodiments concerning target regions identified in Table 2 may be combined with any of the embodiments described above concerning target regions identified in Table 1. In some embodiments, the hypermethylation target regions comprise a plurality of loci listed in Table 1 or Table 2, e.g., at least 10%, 20%, 30%, 40%, 50%, 60%, 70%, 80%, 90%, or 100% of the loci listed in Table 1 or Table 2.

In some embodiments, the hypermethylation target regions comprise regions of one or more genes listed in Table 2b, e.g., at least 5, 10, 20, 30, 40, 50, 60, 70, 80, 90, 100, 150, 200, 250, 300, 350, 400, 450, 500, 550, 600, 650, 700, 750, 800, 850, 900, 950, 1000, 1050,

1000, 1100, 1150 or 1200 genes listed in Table 3. Hypermethylation of these genes can be useful for detecting contributions from immune cells to a DNA sample. In some embodiments, the hypermethylation target regions comprise regions of a plurality of genes listed in Table 2b, e.g., at least 10%, 20%, 30%, 40%, 50%, 60%, 70%, 80%, 90%, or 100% of the genes listed in Table 3. In some embodiments, the hypermethylation target regions comprise regions of all of the genes listed in Table 3.

TABLE 3
Exemplary genes comprising exemplary hypermethylation target regions
GENES

| | | | | | | |
|---|---|---|---|---|---|---|
| CCL5 | RSAD2 | DBNDD1 | CD80 | CD2 | ITPK1 | PRR5 |
| CD19 | ISG15 | SNRPB | CEACAM4 | GOS2 | JAG1 | PTGS2 |
| CD37 | MMP3 | MNAT1 | SH2D1A | MT1G | MMRN2 | PTPN11 |
| CD3D | MARCKSL1 | NUP35 | SAA2-SAA4 | CARD16 | NFIL3 | PVRIG |
| CD3E | IGF2R | TCEB1 | TMEM150B | MT1H | PDGFB | RELA |
| CD3G | LAMB1 | HSPB11 | PYHIN1 | SERPINA1 | PGPEP1 | RICTOR |
| CD3Z | SPARC | C19orf48 | CCL7 | ACP5 | RPL7A | RIPK2 |
| CD79A | FN1 | ID3 | PDCD1 | CXCL5 | SERPINB5 | SPP1 |
| CD79B | ITGA4 | IPO4 | BNC1 | CD74 | SERPINH1 | STAT4 |
| CD8A | SMO | FARSB | ICOS | C1QB | STC1 | TNFRSF14 |
| CD8B1 | MMP19 | EIF4G1 | ITK | HLA-DRB5 | TNFAIP6 | TNFRSF25 |
| IGHG3 | ITGB8 | SKA1 | CLEC4E | FADS3 | VCAN | TNFSF9 |
| IGJ | ITGA5 | MFSD11 | GZMK | IL15RA | VEGFA | TRAT1 |
| IGLC1 | NID1 | PLAUR | UBASH3A | MF12 | VEGFB | TSLP |
| CD14 | TIMP1 | MARVELD2 | CRTAM | IL4I1 | VEGFC | VTCN1 |
| LCK | SEMA3F | MCM3 | EOMES | HLA-A | ATF3 | CCL13 |
| LTB | RHOQ | DHFR | P2RY10 | TM4SF19 | BATF3 | CCL14 |
| MS4A1 | CTNNB1 | RNF41 | NCF1 | MMP9 | CD1C | CCL 18 |
| CORO1A | MMP2 | ID2 | CARD17 | ACY3 | CD36 | CCL19 |
| MNDA | SERPINE1 | H2AFZ | ZBP1 | KRT14 | CD4 | CCL20 |
| CCRL2 | EPHB2 | CDK2 | TNFRSF9 | MT1X | CDC20 | CCL21 |
| SLC7A7 | COL16A1 | NCLN | PDCD1LG2 | MT1M | CTSW | CCL22 |
| HLA-DMA | EPHA2 | ZWILCH | IL12RB1 | IFITM1 | CXCL1 | CCL3/L1 |
| FYB | TNC | DYNLT1 | LILRA6 | IFI35 | DTX3L | CSF1 |
| RNASE6 | JUP | C16orf61 | CXCR3 | BST2 | HLA-DMB | CSF2 |
| TLR2 | ITGA3 | SLC25A40 | SLAMF1 | CD8B | HLA-DOA | CSF2RB |
| CTSC | TCF7L2 | RHOC | SLAMF6 | BATF2 | HLA-DOB | CSF3R |
| LILRB4 | COL3A1 | CCT5 | RHOH | CTSZ | HLA-DPA1 | CX3CL1 |
| PSCDBP | CDH6 | PDIA4 | GPR114 | HAPLN3 | HLA-DPB1 | CX3CR1 |
| CTSS | WNT2B | SNRPA | SLA2 | TAP2 | HLA-DQA1 | CXCL12 |
| RASSF4 | ADAM9 | RBM14 | IL 18RAP | ODF3B | HLA-DQA2 | CXCL14 |
| MSN | DSP | PDLIM7 | GPR171 | SQRDL | IRF8 | CXCL16 |
| CYBB | HSPG2 | PITPNC1 | SCIMP | DOK2 | KIF2C | CXCL2 |
| LAPTM5 | ARHGAP1 | TPM3 | P2RY13 | UBE2L6 | KIR2DL3 | CXCL3 |
| DOCK2 | ITGB5 | CORO1C | LAX1 | SLC15A3 | KIR3DL1 | CXCL6 |
| FCGR1A | IGFBP5 | ERLIN1 | AOAH | SECTM1 | KIR3DL2 | CXCL8 |
| EVI2B | ARHGDIA | PAICS | CXCL11 | GAL | KLRD1 | CXCR2 |
| ADCY7 | LRP1 | TPRKB | CD38 | CXCL13 | MRC1 | GNG4 |
| CD48 | IGFBP2 | SKA2 | GRAP2 | RAC2 | PSMB10 | IL10 |
| ARHGAP1 | CTNNA1 | MYBL1 | WDFY4 | IL1R2 | PSMB5 | IL11 |
| 5 | | | | | | |
| ARRB2 | LRRC17 | SH3BP5L | LTA | LRFN4 | PSMB8 | IL11RA |
| SYK | MMP14 | BRCA2 | GZMH | CLEC4A | PSMC4 | IL12RB2 |
| BTK | NEO1 | SAR1A | PTPN22 | SLAMF8 | THBD | IL16 |
| TNFAIP3 | EFNA5 | POLR3K | IRF4 | SH2D2A | TNF | IL1A |
| FCGR2A | ITGB3 | MRPS28 | GNLY | CDC25B | TRIM21 | IL1B |
| VSIG4 | EPHB3 | NUP107 | CCR5 | PSME2 | UBA7 | IL1RN |
| FPRL2 | CD44 | TUBG1 | SNX20 | GNGT2 | UBB | IL22RA1 |
| IL10RA | IGFBP4 | PNN | LILRB5 | TYROBP | UBE2C | IL24 |
| IFI16 | TNFRSF1A | FAM167A | RGS18 | AIF1 | ULBP2 | IL33 |
| ITGB2 | RAC1 | RFC3 | EMR1 | WARS | VHL | IL34 |
| IL7R | PXN | MYL6 | TIGIT | CD300A | AKT1 | IL6 |
| TBXAS1 | PLAT | MCM7 | SIGLEC7 | C1QC | APC | IL6R |
| FMNL1 | COL8A1 | MAGOHB | GPR65 | ARPC1B | BAD | JAK2 |
| FLI1 | WNT8B | FAM89B | TNFRSF17 | CIITA | BAX | JAK3 |
| RASSF2 | IGFBP3 | TOMM40 | ADAMDEC1 | HAVCR2 | BBC3 | PF4 |
| LYZ | RHOA | CDCA4 | HK3 | SPI1 | BCL2 | PIK3R5 |
| CD163 | EPHB4 | MT3 | LILRB2 | MIR3123 | BCL2L1 | SHC2 |
| CD97 | MMP1 | MTHFD1 | CTLA4 | ACAA2 | BCL6B | STAT2 |
| CCL2 | PAK1 | PSMD 12 | SIGLEC12 | KRTAP19-8 | BID | STAT3 |
| FCGR2B | MTA1 | MYBL2 | CXorf21 | LPCAT1 | BIRC3 | CBLC |
| MERTK | THBS2 | CKLF | SPIB | CARTPT | BIRC5 | CD47 |
| CD84 | CSPG2 | NRIP3 | CLEC4G | ARHGAP26 | BLM | CNTFR |
| CD53 | MMP17 | EZR | CD300C | RPA3 | CASP3 | GHR |
| CD86 | CD59 | C12orf24 | CD300E | MIR129-1 | CASP8 | GZMM |
| HMHA1 | DVL3 | GPLD1 | LILRB1 | FAM150A | CASP9 | IFITM2 |
| CTSL | RHOB | SRM | LAG3 | KCNB2 | CDH1 | IRF9 |
| EVI2A | COL6A3 | RAB3B | HMGA2 | TERF1 | FADD | JAK1 |
| TNFRSF1B | NOTCH2 | NLN | VNN1 | KCNK9 | HMGB1 | KLRB1 |
| CXCR4 | BSG | MT1F | IL2RA | PTK2 | LY96 | KLRK1 |
| LCP1 | MMP11 | TNFRSF12A | NCR3 | GPR20 | RIPK1 | LIF |
| SAMHD1 | COL1A2 | TPI1 | CCR8 | TSNARE1 | RIPK3 | PRLR |
| CPVL | ZYX | HAS2 | TFEC | MIR4475 | ROCK1 | SIRPA |
| HLA-DRB1 | RND3 | APOO | CCR2 | IGFBPL1 | TICAM1 | SPRY4 |
| C13orf18 | THBS1 | FBXO41 | STAP1 | LOC100129316 | TLR3 | DDB2 |
| GIMAP4 | RHOG | MRPL37 | IDO1 | QRFP | TLR4 | ERCC3 |
| SAMSN1 | ICAM1 | GSTCD | MYO1G | CAMSAP1 | TNFRSF10B | FANCA |
| PLCG2 | LAMA4 | SDC1 | SIT1 | GPR37L1 | TNFSF10 | MGMT |
| OSBPL3 | DVL1 | WDR54 | CD209 | CCDC85C | TP53 | NEIL1 |
| RUNX3 | PAK2 | RNF138 | LILRA5 | GACAT2 | BNIP3 | PARP4 |
| FCGR3A | COL6A1 | APITD1 | SIGLEC1 | LINC01443 | DEPTOR | POLR2A |
| AMPD3 | FGD1 | RMND5B | UBD | CDH2 | HIF1A | TNKS |
| MYO1F | CEP78 | ENO1 | SPN | MIR4436A | HRAS | UBE2T |
| CECR1 | LSM3 | MAP3K8 | CXCL9 | INSM1 | KRAS | XCL1 |
| LYN | LRRC40 | TMEM130 | CD70 | LOC101929625 | MAP3K7 | XCL2 |
| MPP1 | STK17A | SNX17 | GZMB | COL18A1 | MAPK10 | ARID1A |
| LRMP | RPN1 | KRR1 | SIGLEC10 | OPA1-AS1 | MTOR | BRD3 |
| FGL2 | JUNB | TAGLN | CD300LF | C9 | NRAS | BRD4 |
| NCKAP1L | NUP85 | PA2G4 | CCL8 | MIR585 | PIK3CA | DNMT1 |
| HCLS1 | FLNC | RUVBL1 | PTPN7 | MAD1L1 | PIK3CD | HDAC11 |
| SELL | HMGN2 | SNRPD1 | CCL4 | CPA2 | PIK3R1 | HDAC3 |
| CASP1 | RPP40 | LOXL2 | CMKLR1 | LOC100130298 | PIK3R2 | HDAC4 |
| SELPLG | UQCR10 | POLE2 | ICAM3 | C8orf34 | PRKAA2 | HDAC5 |
| CD33 | AIMP2 | MAPRE1 | SIRPG | OC90 | PRKACB | HELLS |
| GPNMB | CHEK1 | IMP4 | NLRP3 | TRAPPC9 | PTEN | HMGA1 |
| NCF2 | VTA1 | EMP2 | MARCO | MED27 | RPS6KB1 | KAT2B |
| FNBP1 | EXOSC8 | PSMD2 | CCL4L1 | MLH1 | RPTOR | MAP3K12 |
| IL18 | CENPO | MET | CD180 | MSH2 | ANLN | BMP2 |
| B2N | PNO1 | IFRD2 | SIGLEC9 | MSH6 | ATM | GAS1 |
| SP140 | SLC16A1 | LMNB2 | KRT6B | PMS2 | CCNA1 | GLI1 |
| FCER1G | WDR77 | PLOD2 | NFAM1 | BRCA1 | CCNB1 | IHH |
| LCP2 | UBE2J1 | NCEH1 | GPR84 | TERT | CCND1 | PRKX |
| LY86 | NOP16 | NME1 | KLK5 | EphA2 | CCND3 | WNT10A |
| LAIR1 | NUDT1 | STRA13 | CCL23 | cGAS | CCNO | WNT11 |
| IFI30 | SMC2 | ACTL6A | ZBED2 | STING | CDC25C | WNT2 |
| TNFSF13B | SLC25A5 | DLEU1 | CD28 | CD8 | CDK6 | WNT3A |
| LST1 | NUPL1 | SNRPA1 | CEACAM21 | PDL2 | CDKN1A | WNT4 |
| FGR | DLEU2 | CBX1 | PRF1 | ASXL3 | CDKN1C | WNT5A |
| NPL | PDAP1 | LYAR | CSF3 | SPEDF | CDKN2A | WNT5B |
| PLEK | CCBL2 | PTPLB | KIAA0226L | PSMA | CDKN2B | WNT7B |
| PTPRC | COX17 | PFN1 | CXCR6 | GPC3 | CENPF | ALDOA |
| GNPTAB | BCCIP | CENPJ | CCL24 | RAD51 | CEP55 | EGF |
| SLC1A3 | PLG | COTL1 | ARHGAP9 | ACVR1 | EXO1 | EGFR |
| HCK | RGS8 | SPRYD7 | LILRB3 | APLNR | H2AFX | ERBB2 |
| NPC2 | SNRPC | USPL1 | VNN2 | CDC27 | MELK | HK1 |
| C3AR1 | PLK4 | MRPL12 | ZAP70 | CDK4 | MKI67 | HK2 |
| PIK3CG | NUTF2 | ADAMTS1 | TNFSF8 | HLA-C | MYC | IFNGR1 |
| DAPK1 | LSM4 | GLRX3 | HEPHL1 | HLA-E | NBN | IFNGR2 |
| ALOX5AP | SMS | WSB2 | CCL3 | HLA-G | POLD1 | LDHA |
| CSF1R | EBNA1BP2 | MRPS16 | CLEC10A | IL1R | PRKCA | NOS2 |
| CUGBP2 | C13orf27 | DCLRE1B | CCL3L3 | IL2RB | RAD50 | PDK1 |
| APOE | VDAC1 | MKKS | CCR4 | INFGR1 | RAD51C | PFKFB3 |
| APOC1 | PSMD14 | C3orf26 | ITGAL | INFGR2 | RB1 | SLC2A1 |
| CD52 | MYCBP | CPEB4 | SAA2 | IRF1 | RBL2 | TFRC |
| LHFPL2 | SMURF2 | SPAG17 | GZMA | IRF2 | RRM2 | CD276 |
| C1orf54 | GNG11 | MLF1IP | BCL2A1 | KIR2DS2 | TYMS | CD6 |
| IKZF1 | F3 | UAP1 | RNASE2 | KIR2DS3 | ADORA2A | CDH5 |
| SH2B3 | BRIP1 | COQ2 | SAA1 | PIAS4 | BTLA | CLEC14A |
| WIPF1 | HNRNPA2B1 | WDHD1 | NKG7 | PTPN2 | CD247 | CLEC5A |
| IFI44 | DCK | DCBLD2 | MT2A | SERPINB3 | CD27 | CLEC7A |
| IFI44L | ALKBH7 | KIAA0090 | FAM26F | SERPINB4 | CD274 | CLECL1 |
| DDX58 | HN1L | SAR1B | PSMB9 | SPOP | CD40 | ICAM2 |
| IFI6 | TPM1 | PSMA7 | TYMP | TAPBP | CD40LG | ICAM5 |
| IFI27 | HYLS1 | PSMC3 | CD7 | TGFBR1 | CD69 | ITGA1 |
| IFIT2 | HAUS1 | COPS6 | SYT12 | CD58 | CHUK | ITGA2 |
| IFIT1 | NUP93 | DUT | S100A7 | ANGPT1 | DPP4 | ITGAE |
| IFIT3 | SNRPE | PPIH | HLA-F | ANGPT2 | EGR1 | ITGAM |
| CXCL10 | ITGA6 | PHF19 | MT1E | ANGPTL4 | FYN | ITGAX |
| MX1 | CENPN | TPM2 | ISG20 | CCND2 | ICOSLG | NCAM1 |
| OAS1 | C11orf24 | MCTS1 | C1QA | CCNE1 | IKBKB | NECTIN1 |
| OAS2 | GGH | EIF4EBP1 | TNFRSF18 | CES3 | IKBKG | PECAM1 |
| OAS3 | PFKP | HNRNPR | IL32 | DLL4 | IL15 | PVR |
| HERC5 | FARSA | IFNG | SOCS1 | E2F3 | IL17A | SELE |
| SAMD9 | EIF2AK1 | TLR8 | HLA-DQB1 | EDN1 | IL18R1 | SELP |
| HERC6 | CENPW | FASLG | IFITM3 | EZH2 | IL2 | THY1 |
| DDX60 | TUBA4A | KIR2DL4 | PI3 | FGF18 | IL4 | VCAM1 |
| RTP4 | TRA2B | IL21R | HCST | FGFR1 | LY9 | EIF2AK2 |
| IFIH1 | UMPS | GBP5 | SUSD3 | FLT1 | NECTIN2 | FLNB |
| STAT1 | MRTO4 | ZNF683 | HLA-B | FSTL3 | NFATC2 | GBP1 |
| TAP1 | NUDT15 | PLA2G2D | IL2RG | HEY1 | NFKB1 | GBP2 |
| OASL | PGM2 | TBX21 | HLA-DRA | ITGAV | NFKBIA | GBP4 |
| TPSD1 | GATA2 | SERPINF1 | FCER1A | DAPP1 | CCR7 | EREG |
| SLC18A2 | HPGDS | PLAC8 | CD1A | C10ORF54 | XCR1 | CD81 |
| IL1RL1 | TCF4 | JCHAIN | AXL | ANPEP | MTSS1 | PRDM1 |
| VWA5A | MZB1 | SLC15A4 | CD1E | CST7 | MSA4A7 | ABL2 |
| TPSAB2 | IL3RA | ITM2C | PKIB | FLT3 | LILRA1 | SDC4 |
| IFNA1 | PDGFA | MMP7 | CRABP2 | TNFRSF4 | FZD9 | PNOC |
| IFNAR1 | PDGFRB | NID2 | DAB2 | TNFSF12 | GPC4 | HLA-DR |
| IRF3 | PLA1A | RELN | FCAR | TNFSF13 | SFRP1 | LIN |
| IRF5 | PLA2G2A | AQP9 | FCN1 | TNFSF18 | SFRP4 | CD11b |
| IRF7 | RASAL1 | CEBPB | FOSL1 | TNFSF4 | SOX11 | CD15 |
| BLK | RASGRF1 | ERO1A | FPR1 | APH1B | SOX2 | CD11c |
| CD5 | RELB | FBP1 | FPR3 | DLL1 | FOXP3 | CD34 |
| CD96 | TGFB1 | G6PD | IER3 | DTX4 | NCR1 | CD83 |
| F2RL1 | TGFB2 | GLS | OLR1 | HES1 | CEACAM3 | PD-L1 |
| SLAMF7 | TGFB3 | GOT1 | PDZK1IP1 | JAG2 | FCGR3A/B | CD45 |
| DUSP1 | TGFBR2 | GOT2 | S100A12 | MAML2 | SIGLEC5 | CD124 |
| DUSP2 | A2M | LDHB | S100A8 | MFNG | CPA3 | CD66 |
| DUSP5 | COL11A1 | PC | S100A9 | NOTCH1 | HDC | CD3 |
| FAS | COL11A2 | PCK2 | SIGLEC8 | TCL1A | MS4A2 | CD13 |
| FGF13 | COL17A1 | PFKM | SIRPB2 | ACVR1C | TPSAB1/B2 | TPSAB1 |
| FGF9 | COL4A5 | PKM | SLC11A1 | BAMBI | CD68 | LILRA4 |
| KDR | COL5A1 | SGK1 | TLR1 | ID4 | MS4A4A | CD16 |
| KIT | COMP | SLC1A5 | TREM1 | INHBA | CD244 | PLTP |
| MAP3K5 | LAMA1 | SLC7A5 | TREM2 | SMAD5 | PTGER4 | ADM |
| NF1 | LAMB3 | AREG | NFKBIE | AXIN1 | HSD11B1 | CSTB |
| NFKB2 | LAMC2 | ARG1 | TNFRSF11A | DKK1 | FAM30A | PPARG |
| NGFR | LTBP1 | C5AR1 | TNFRSF11B | FZD8 | FCRL2 | RGCC |

Additional hypermethylation target regions may be obtained, e.g., from the Cancer Genome Atlas. Kang et al., Genome Biology 18:53 (2017), describe construction of a probabilistic method called CancerLocator using hypermethylation target regions from breast, colon, kidney, liver, and lung. In some embodiments, the hypermethylation target regions can be specific to one or more types of cancer. Accordingly, in some embodiments, the hypermethylation target regions include one, two, three, four, or five subsets of hypermethylation target regions that collectively show hypermethylation in one, two, three, four, or five of breast, colon, kidney, liver, and lung cancers.

In some embodiments, where different epigenetic target regions are captured from first and second subsamples, the epigenetic target regions captured from the first subsample comprise hypermethylation target regions.

b. Hypomethylation Target Regions

In some embodiments, the epigenetic target region set comprises one or more hypomethylation target regions. In some embodiments, hypomethylation target regions are exclusively hypomethylated in one immune cell type or hypomethylated to a greater extent in one immune cell type than in any other immune cell type or in any other immune cell type within the same immune cell cluster. In some such embodiments, hypomethylation target regions indicate the levels of particular immune cell types from which the DNA originated, including rare immune cell types such as activated B cells (including memory B cells and plasma cells), activated T cells (including regulatory T cells (Tregs), CD4 effector memory T cells, CD4 central memory T cells, CD8 effector memory T cells, and CD8 central memory T cells), and natural killer (NK) cells. Methylation patterns of hypomethylation target regions that are useful for deconvoluting immune cell types may further change in certain disease states, such as cancer. Thus, in some embodiments, hypomethylation target regions that are useful for deconvoluting immune cell types are also useful for determining the likelihood that the subject from which the sample was obtained has cancer or precancer. In some such embodiments, hypomethylation target regions are useful for determining whether levels of particular immune cell types are abnormal and whether such abnormal levels are likely related to the presence of cancer or precancer, or if they are related to a different disease or condition other than cancer or precancer.

Additionally, global hypomethylation is a commonly observed phenomenon in various cancers. See, e.g., Hon et al., Genome Res. 22:246-258 (2012) (breast cancer); Ehrlich, Epigenomics 1:239-259 (2009) (review article noting observations of hypomethylation in colon, ovarian, prostate, leukemia, hepatocellular, and cervical cancers). For example, regions such as repeated elements, e.g., LINE1 elements, Alu elements, centromeric tandem repeats, pericentromeric tandem repeats, and satellite DNA, and intergenic regions that are ordinarily methylated in healthy cells may show reduced methylation in tumor cells. Accordingly, in some embodiments, the epigenetic target region set includes hypomethylation target regions in which a decrease in the level of observed methylation indicates an increased likelihood of the presence of cancer. Detection of such hypomethylation target regions, e.g., in conjunction with detection of hypomethylation target regions indicative of immune cell types, may further increase the specificity and/or sensitivity of methods described herein. In another example, as discussed above, hypomethylation target regions can include regions that do not necessarily differ in methylation in cancerous tissue relative to DNA from healthy tissue of the same type, but do differ in methylation (e.g., are less methylated) relative to cfDNA that is typical in healthy subjects. Where, for example, the presence of a cancer results in increased cell death such as apoptosis of cells of the tissue type corresponding to the cancer, such a cancer can be detected at least in part using such hypomethylation target regions. In some embodiments, hypomethylation target regions useful for determining the likelihood that a subject has cancer are different than the hypomethylation target regions useful for determining the levels of particular immune cell types. In some embodiments, at least some of the hypomethylation target regions useful for determining the likelihood that a subject has cancer are the same as the hypomethylation variable target regions useful for determining the levels of particular immune cell types.

In some embodiments, hypomethylation target regions include repeated elements and/or intergenic regions. In some embodiments, repeated elements include one, two, three, four, or five of LINE1 elements, Alu elements, centromeric tandem repeats, pericentromeric tandem repeats, and/or satellite DNA.

Exemplary specific genomic regions that show cancer-associated hypomethylation include nucleotides 8403565-8953708 and 151104701-151106035 of human chromosome 1. In some embodiments, the hypomethylation target regions overlap or comprise one or both of these regions.

Additionally, hypomethylation target regions may be obtained, e.g., from Fox-Fisher et al., ElifeNov 29; 10 (2021), EpiDISH R package, Moss et al., Nat Commun 9:1 (2018), and Loyfer et al. bioRxiv https://doi.org/10.1101/2022.01.24.477547 (2022). In some embodiments, the hypomethylation target regions can be specific to one or more types of immune cells.

In some embodiments, where different epigenetic target regions are captured from first and second subsamples, the epigenetic target regions captured from the second subsample comprise hypomethylation target regions. In some embodiments, the epigenetic target regions captured from the second subsample comprise hypomethylation target regions and the epigenetic target regions captured from the first subsample comprise hypermethylation target regions.

c. CTCF Binding Regions

CTCF is a DNA-binding protein that contributes to chromatin organization and often colocalizes with cohesin. Perturbation of CTCF binding sites has been reported in a variety of different cancers. See, e.g., Katainen et al., Nature Genetics, doi:10.1038/ng.3335, published online 8 Jun. 2015; Guo et al., Nat. Commun. 9:1520 (2018). CTCF binding results in recognizable patterns in cfDNA that can be detected by sequencing, e.g., through fragment length analysis. Details regarding sequencing-based fragment length analysis are provided in Snyder et al., Cell 164:57-68 (2016); WO 2018/009723; and US20170211143A1, each of which are incorporated herein by reference.

Thus, perturbations of CTCF binding result in variation in the fragmentation patterns of cfDNA. As such, CTCF binding sites are a type of fragmentation variable target regions.

There are many known CTCF binding sites. See, e.g., the CTCFBSDB (CTCF Binding Site Database), available on the Internet at insulatordb.uthsc.edu/; Cuddapah et al., Genome Res. 19:24-32 (2009); Martin et al., Nat. Struct. Mol. Biol. 18:708-14 (2011); Rhee et al., Cell. 147:1408-19 (2011), each of which are incorporated by reference. Exemplary CTCF binding sites are at nucleotides 56014955-56016161 on chromosome 8 and nucleotides 95359169-95360473 on chromosome 13.

Accordingly, in some embodiments, the epigenetic target region set includes CTCF binding regions. In some embodiments, the CTCF binding regions comprise at least 10, 20, 50, 100, 200, or 500 CTCF binding regions, or 10-20, 20-50, 50-100, 100-200, 200-500, or 500-1000 CTCF binding regions, e.g., such as CTCF binding regions described above or in one or more of CTCFBSDB or the Cuddapah et al., Martin et al., or Rhee et al. articles cited above.

In some embodiments, at least some of the CTCF sites can be methylated or unmethylated, wherein the methylation state is correlated with the whether or not the cell is a cancer cell. In some embodiments, the epigenetic target region set comprises at least 100 bp, at least 200 bp, at least 300 bp, at least 400 bp, at least 500 bp, at least 750 bp, at least 1000 bp upstream and downstream regions of the CTCF binding sites.

d. Transcription Start Sites

Transcription start sites may also show perturbations in neoplastic cells. For example, nucleosome organization at various transcription start sites in healthy cells of the hematopoietic lineage—which contributes substantially to cfDNA in healthy individuals—may differ from nucleosome organization at those transcription start sites in neoplastic cells. This results in different cfDNA patterns that can be detected by sequencing, as discussed generally in Snyder et al., Cell 164:57-68 (2016); WO 2018/009723; and US20170211143A1. In another example, transcription start sites may not necessarily differ epigenetically in cancerous tissue relative to DNA from healthy tissue of the same type, but do differ epigenetically (e.g., with respect to nucleosome organization) relative to cfDNA that is typical in healthy subjects. Where, for example, the presence of a cancer results in increased cell death, such as apoptosis, of cells of the tissue type corresponding to the cancer, such a cancer can be detected at least in part using such differences in transcription start sites.

Thus, perturbations of transcription start sites also result in variation in the fragmentation patterns of cfDNA. As such, transcription start sites are also a type of fragmentation variable target regions.

Human transcriptional start sites are available from DBTSS (DataBase of Human Transcription Start Sites), available on the Internet at dbtss.hgc.jp and described in Yamashita et al., Nucleic Acids Res. 34(Database issue): D86-D89 (2006), which is incorporated herein by reference.

Accordingly, in some embodiments, the epigenetic target region set includes transcriptional start sites. In some embodiments, the transcriptional start sites comprise at least 10, 20, 50, 100, 200, or 500 transcriptional start sites, or 10-20, 20-50, 50-100, 100-200, 200-500, or 500-1000 transcriptional start sites, e.g., such as transcriptional start sites listed in DBTSS. In some embodiments, at least some of the transcription start sites can be methylated or unmethylated, wherein the methylation state is correlated with whether or not the cell is a cancer cell. In some embodiments, the epigenetic target region set comprises at least 100 bp, at least 200 bp, at least 300 bp, at least 400 bp, at least 500 bp, at least 750 bp, at least 1000 bp upstream and downstream regions of the transcription start sites.

e. Focal Amplifications

Although focal amplifications are somatic mutations, they can be detected by sequencing based on read frequency in a manner analogous to approaches for detecting certain epigenetic changes such as changes in methylation. As such, regions that may show focal amplifications in cancer can be included in the epigenetic target region set and may comprise one or more of AR, BRAF, CCND1, CCND2, CCNE1, CDK4, CDK6, EGFR, ERBB2, FGFR1, FGFR2, KIT, KRAS, MET, MYC, PDGFRA, PIK3CA, and RAF1. For example, in some embodiments, the epigenetic target region set comprises at least 2, 3, 4, 5, 6, 7, 8, 9, 10, 11, 12, 13, 14, 15, 16, 17, or 18 of the foregoing targets.

f. Methylation Control Regions or Control Regions

It can be useful to include control regions to facilitate data validation. In some embodiments, the epigenetic target region set includes control regions that are expected to be methylated or unmethylated in essentially all samples, regardless of whether the DNA is derived from a cancer cell or a normal cell. In some embodiments, the epigenetic target region set includes negative control regions that are expected to be hypomethylated or unmethylated in essentially all samples. In some embodiments, the epigenetic target region set includes positive control regions that are expected to be hypermethylated in essentially all samples.

### 2. Sequence-Variable Target Region Set

In some embodiments, the sequence-variable target region set comprises a plurality of regions known to undergo somatic mutations (e.g., single nucleotide variations and/or indels) in cancer. The single nucleotide variations and/or indels may be relative to a reference sequence, e.g., a published human genome sequence, such as the GRCh38 human genome assembly.

In some aspects, the sequence-variable target region set targets a plurality of different genes or genomic regions (“panel”) selected such that a determined proportion of subjects having a cancer exhibits a genetic variant or tumor marker in one or more different genes or genomic regions in the panel. The panel may be selected to limit a region for sequencing to a fixed number of base pairs. The panel may be selected to sequence a desired amount of DNA, e.g., by adjusting the affinity and/or amount of the probes as described elsewhere herein. The panel may be further selected to achieve a desired sequence read depth. The panel may be selected to achieve a desired sequence read depth or sequence read coverage for an amount of sequenced base pairs. The panel may be selected to achieve a theoretical sensitivity, a theoretical specificity, and/or a theoretical accuracy for detecting one or more genetic variants in a sample. [0284] Probes for detecting the panel of regions can include those for detecting genomic regions of interest (hotspot regions). Information about chromatin structure can be taken into account in designing probes, and/or probes can be designed to maximize the likelihood that particular sites (e.g., KRAS codons 12 and 13) can be captured, and may be designed to optimize capture based on analysis of cfDNA coverage and fragment size variation impacted by nucleosome binding patterns and GC sequence composition. Regions used herein can also include non-hotspot regions optimized based on nucleosome positions and GC models.

Examples of listings of genomic locations of interest may be found in Table 4 and Table 5. In some embodiments, a sequence-variable target region set used in the methods of the present disclosure comprises at least a portion of at least 5, at least 10, at least 15, at least 20, at least 25, at least 30, at least 35, at least 40, at least 45, at least 50, at least 55, at least 60, at least 65, or 70 of the genes of Table 3. In some embodiments, a sequence-variable target region set used in the methods of the present disclosure comprises at least 5, at least 10, at least 15, at least 20, at least 25, at least 30, at least 35, at least 40, at least 45, at least 50, at least 55, at least 60, at least 65, or 70 of the SNVs of Table 4. In some embodiments, a sequence-variable target region set used in the methods of the present disclosure comprises at least 1, at least 2, at least 3, at least 4, at least 5, or 6 of the fusions of Table 4. In some embodiments, a sequence-variable target region set used in the methods of the present disclosure comprise at least a portion of at least 1, at least 2, or 3 of the indels of Table 4. In some embodiments, a sequence-variable target region set used in the methods of the present disclosure comprises at least a portion of at least 5, at least 10, at least 15, at least 20, at least 25, at least 30, at least 35, at least 40, at least 45, at least 50, at least 55, at least 60, at least 65, at least 70, or 73 of the genes of Table 5. In some embodiments, a sequence-variable target region set used in the methods of the present disclosure comprises at least 5, at least 10, at least 15, at least 20, at least 25, at least 30, at least 35, at least 40, at least 45, at least 50, at least 55, at least 60, at least 65, at least 70, or 73 of the SNVs of Table 5. In some embodiments, a sequence-variable target region set used in the methods of the present disclosure comprises at least 1, at least 2, at least 3, at least 4, at least 5, or 6 of the fusions of Table 5. In some embodiments, a sequence-variable target region set used in the methods of the present disclosure comprises at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the indels of Table 5. Each of these genomic locations of interest may be identified as a backbone region or hot-spot region for a given panel. An example of a listing of hot-spot genomic locations of interest may be found in Table 6. In some embodiments, a sequence-variable target region set used in the methods of the present disclosure comprises at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 of the genes of Table 6. Each hot-spot genomic region is listed with several characteristics, including the associated gene, chromosome on which it resides, the start and stop position of the genome representing the gene's locus, the length of the gene's locus in base pairs, the exons covered by the gene, and the critical feature (e.g., type of mutation) that a given genomic region of interest may seek to capture.

TABLE 4

| Point Mutations (SNVs) | | | | Fusions |
|---|---|---|---|---|
| AKT1 | ROS1 | PDGFRA | NOTCH1 | ALK |
| ATM | TP53 | RET | PTEN | FGFR2 |
| CCNE1 | APC | SMO | RHOA | FGFR3 |
| CTNNB1 | BRCA1 | VHL | STK11 | NTRK1 |
| FGFR1 | CDK4 | ARAF | | RET |
| GNAS | ERBB2 | CCND1 | | ROS1 |
| JAK3 | FGFR3 | CDKN2A | | |
| MLH1 | HRAS | EZH2 | | |
| NPM1 | KRAS | GNA11 | | |
| PTPN11 | MYC | IDH2 | | |
| RIT1 | NTRK1 | MAP2K2 | | |
| TERT | RB1 | NFE2L2 | | |
| ALK | SMAD4 | PIK3CA | | |
| BRAF | TSC1 | RHEB | | |
| CDH1 | AR | SRC | | |
| EGFR | BRCA2 | ARIDIA | | |
| FGFR2 | CDK6 | CCND2 | | |
| HNF1A | ESR1 | CDKN2B | | |
| KIT | GATA3 | FBXW7 | | |
| MPL | IDH1 | GNAQ | | |
| NRAS | MAP2K1 | JAK2 | | |
| RAF1 | NF1 | MET | | |

TABLE 5

| Point Mutations (SNVs) | | | Fusions |
|---|---|---|---|
| AKT1 | ARAF | CCND1 | FGFR2 |
| ATM | BRCA2 | CDKN2A | FGFR3 |
| CCNE1 | CDK6 | EZH2 | NTRK1 |
| CTNNB1 | ESR1 | GNA11 | RET |
| FGFR1 | GATA3 | IDH2 | ROS1 |
| GNAS | IDH1 | MAP2K2 | |
| JAK3 | MAP2K1 | NFE2L2 | |
| MLH1 | NF1 | PIK3CA | |
| NPM1 | PDGFRA | RHEB | |
| PTPN11 | RET | MAPK1 | |
| RIT1 | SMO | MAPK3 | |
| TERT | VHL | DDR2 | |
| NTRK3 | ARIDIA | FBXW7 | |
| ALK | CCND1 | GNAQ | |
| BRAF | CDKN2A | JAK2 | |
| CDH1 | EZH2 | MET | |
| EGFR | GNA11 | NOTCH1 | |
| FGFR2 | IDH2 | PTEN | |
| HNF1A | MAP2K2 | RHOA | |
| KIT | NFE2L2 | STK11 | |
| MPL | PIK3CA | MTOR | |
| NRAS | RHEB | | |
| RAF1 | MAPK1 | | |
| ROS1 | MAPK3 | | |
| TP53 | CCND2 | | |

TABLE 6

| | | Start | Stop | Length | Exons | |
|---|---|---|---|---|---|---|
| Gene | Chromosome | Position | Position | (bp) | Covered | Critical Feature |
| ALK | chr2 | 29446405 | 29446655 | 250 | intron 19 | Fusion |
| ALK | chr2 | 29446062 | 29446197 | 135 | intron 20 | Fusion |
| ALK | chr2 | 29446198 | 29446404 | 206 | 20 | Fusion |
| ALK | chr2 | 29447353 | 29447473 | 120 | intron 19 | Fusion |
| ALK | chr2 | 29447614 | 29448316 | 702 | intron 19 | Fusion |
| ALK | chr2 | 29448317 | 29448441 | 124 | 19 | Fusion |
| ALK | chr2 | 29449366 | 29449777 | 411 | intron 18 | Fusion |
| ALK | chr2 | 29449778 | 29449950 | 172 | 18 | Fusion |
| BRAF | chr7 | 140453064 | 140453203 | 139 | 15 | BRAF V600 |
| CTNNB1 | chr3 | 41266007 | 41266254 | 247 | 3 | S37 |
| EGFR | chr7 | 55240528 | 55240827 | 299 | 18 and 19 | G719 and deletions |
| EGFR | chr7 | 55241603 | 55241746 | 143 | 20 | Insertions/T790M |
| EGFR | chr7 | 55242404 | 55242523 | 119 | 21 | L858R |
| ERBB2 | chr17 | 37880952 | 37881174 | 222 | 20 | Insertions |
| ESR1 | chr6 | 152419857 | 152420111 | 254 | 10 | V534, P535, L536, |
| | | | | | | Y537, D538 |
| FGFR2 | chr10 | 123279482 | 123279693 | 211 | 6 | S252 |
| GATA3 | chr10 | 8111426 | 8111571 | 145 | 5 | SS/Indels |
| GATA3 | chr10 | 8115692 | 8116002 | 310 | 6 | SS/Indels |
| GNAS | chr20 | 57484395 | 57484488 | 93 | 8 | R844 |
| IDH1 | chr2 | 209113083 | 209113394 | 311 | 4 | R132 |
| IDH2 | chr15 | 90631809 | 90631989 | 180 | 4 | R140, R172 |
| KIT | chr4 | 55524171 | 55524258 | 87 | 1 | |
| KIT | chr4 | 55561667 | 55561957 | 290 | 2 | |
| KIT | chr4 | 55564439 | 55564741 | 302 | 3 | |
| KIT | chr4 | 55565785 | 55565942 | 157 | 4 | |
| KIT | chr4 | 55569879 | 55570068 | 189 | 5 | |
| KIT | chr4 | 55573253 | 55573463 | 210 | 6 | |
| KIT | chr4 | 55575579 | 55575719 | 140 | 7 | |
| KIT | chr4 | 55589739 | 55589874 | 135 | 8 | |
| KIT | chr4 | 55592012 | 55592226 | 214 | 9 | |
| KIT | chr4 | 55593373 | 55593718 | 345 | 10 and 11 | 557, 559, 560, 576 |
| KIT | chr4 | 55593978 | 55594297 | 319 | 12 and 13 | V654 |
| KIT | chr4 | 55595490 | 55595661 | 171 | 14 | T670, S709 |
| KIT | chr4 | 55597483 | 55597595 | 112 | 15 | D716 |
| KIT | chr4 | 55598026 | 55598174 | 148 | 16 | L783 |
| KIT | chr4 | 55599225 | 55599368 | 143 | 17 | C809, R815, D816, |
| | | | | | | L818, D820, S821F, |
| | | | | | | N822, Y823 |
| KIT | chr4 | 55602653 | 55602785 | 132 | 18 | A829P |
| KIT | chr4 | 55602876 | 55602996 | 120 | 19 | |
| KIT | chr4 | 55603330 | 55603456 | 126 | 20 | |
| KIT | chr4 | 55604584 | 55604733 | 149 | 21 | |
| KRAS | chr12 | 25378537 | 25378717 | 180 | 4 | A146 |
| KRAS | chr12 | 25380157 | 25380356 | 199 | 3 | Q61 |
| KRAS | chr12 | 25398197 | 25398328 | 131 | 2 | G12/G13 |
| MET | chr7 | 116411535 | 116412255 | 720 | 13, 14, | MET exon 14 SS |
| | | | | | intron 13, | |
| | | | | | intron 14 | |
| NRAS | chr1 | 115256410 | 115256609 | 199 | 3 | Q61 |
| NRAS | chr1 | 115258660 | 115258791 | 131 | 2 | G12/G13 |
| PIK3CA | chr3 | 178935987 | 178936132 | 145 | 10 | E545K |
| PIK3CA | chr3 | 178951871 | 178952162 | 291 | 21 | H1047R |
| PTEN | chr10 | 89692759 | 89693018 | 259 | 5 | R130 |
| SMAD4 | chr18 | 48604616 | 48604849 | 233 | 12 | D537 |
| TERT | chr5 | 1294841 | 1295512 | 671 | promoter | chr5:1295228 |
| TP53 | chr17 | 7573916 | 7574043 | 127 | 11 | Q331, R337, R342 |
| TP53 | chr17 | 7577008 | 7577165 | 157 | 8 | R273 |
| TP53 | chr17 | 7577488 | 7577618 | 130 | 7 | R248 |
| TP53 | chr17 | 7578127 | 7578299 | 172 | 6 | R213/Y220 |
| TP53 | chr17 | 7578360 | 7578564 | 204 | 5 | R175/Deletions |
| TP53 | chr17 | 7579301 | 7579600 | 299 | 4 | |
| | | | | 12574 | | |
| | | | | (total | | |
| | | | | target | | |
| | | | | region) | | |
| | | | | 16330 | | |
| | | | | (total | | |
| | | | | probe | | |
| | | | | coverage) | | |

Additionally, or alternatively, suitable target region sets are available from the literature. For example, Gale et al., PLoS One 13: e0194630 (2018), which is incorporated herein by reference, describes a panel of 35 cancer-related gene targets that can be used as part or all of a sequence-variable target region set. These 35 targets are AKTI, ALK, BRAF, CCND1, CDK2A, CTNNB1, EGFR, ERBB2, ESR1, FGFR1, FGFR2, FGFR3, FOXL2, GAT A3, GNA11, GNAQ, GNAS, HRAS, IDH1, IDH2, KIT, KRAS, MED 12, MET, MYC, NFE2L2, NRAS, PDGFRA, PIK3CA, PPP2R1A, PTEN, RET, STK11, TP53, and U2AF1.

In some embodiments, the sequence-variable target region set comprises target regions from at least 10, 20, 30, or 35 cancer-related genes, such as the cancer-related genes listed above.

### G. Subjects

In some embodiments, the DNA (e.g., cfDNA) is obtained from a subject having a cancer or a precancer, an infection, transplant rejection, or other disease directly or indirectly affecting the immune system. In some embodiments, the DNA (e.g., cfDNA) is obtained from a subject suspected of having a cancer or a precancer, an infection, transplant rejection, or other disease directly or indirectly affecting the immune system. In some embodiments, the DNA (e.g., cfDNA) is obtained from a subject having a tumor. In some embodiments, the DNA (e.g., cfDNA) is obtained from a subject suspected of having a tumor. In some embodiments, the DNA (e.g., cfDNA) is obtained from a subject having neoplasia. In some embodiments, the DNA (e.g., cfDNA) is obtained from a subject suspected of having neoplasia. In some embodiments, the DNA (e.g., cfDNA) is obtained from a subject in remission from a tumor, cancer, or neoplasia (e.g., following chemotherapy, surgical resection, radiation, or a combination thereof). In any of the foregoing embodiments, the cancer, tumor, or neoplasia or suspected cancer, tumor, or neoplasia may be of the lung, colon, rectum, kidney, breast, prostate, or liver. In some embodiments, the cancer, tumor, or neoplasia or suspected cancer, tumor, or neoplasia is of the lung. In some embodiments, the cancer, tumor, or neoplasia or suspected cancer, tumor, or neoplasia is of the colon or rectum. In some embodiments, the cancer, tumor, or neoplasia or suspected cancer, tumor, or neoplasia is of the breast. In some embodiments, the cancer, tumor, or neoplasia or suspected cancer, tumor, or neoplasia is of the prostate. In any of the foregoing embodiments, the subject may be a human subject.

H. Pooling of DNA from Samples or Subsamples or Portions Thereof

In some embodiments, the methods herein comprise preparing one or more pools comprising tagged DNA from a plurality of partitioned subsamples. In some embodiments, a pool comprises at least a portion of the DNA of a hypomethylated partition and at least a portion of the DNA of a hypermethylated partition. Target regions, e.g., including epigenetic target regions and/or sequence-variable target regions, may be captured from a pool. The steps of capturing a target region set from at least an aliquot or portion of a sample or subsample described elsewhere herein encompass capture steps performed on a pool comprising DNA from first and second subsamples. A step of amplifying DNA in a pool may be performed before capturing target regions from the pool. The capturing step may have any of the features described for capturing steps elsewhere herein.

In some embodiments, the methods comprise preparing a first pool comprising at least a portion of the DNA of a hypomethylated partition. In some embodiments, the methods comprise preparing a second pool comprising at least a portion of the DNA of a hypermethylated partition. In some embodiments, the methods comprise capturing at least a first set of target regions from the first pool, wherein the first set comprises sequence-variable target regions. A step of amplifying DNA in the first pool may be performed before this capture step. In some embodiments, capturing the first set of target regions from the first pool comprises contacting the DNA of the first pool with a first set of target-specific probes, wherein the first set of target-specific probes comprises target-binding probes specific for the sequence-variable target regions. In some embodiments, the methods comprise capturing a second plurality of sets of target regions from the second pool, wherein the second plurality comprises sequence-variable target regions and epigenetic target regions. A step of amplifying DNA in the second pool may be performed before this capture step. In some embodiments, capturing the second plurality of sets of target regions from the second pool comprises contacting the DNA of the first pool with a second set of target-specific probes, wherein the second set of target-specific probes comprises target-binding probes specific for the sequence-variable target regions and target-binding probes specific for the epigenetic target regions.

In some embodiments, sequence-variable target regions are captured from a second portion of a partitioned subsample. The second portion may include some, a majority, substantially all, or all of the DNA of the subsample that was not included in the pool. The regions captured from the pool and from the subsample may be combined and analyzed in parallel.

The epigenetic target regions may show differences in methylation levels and/or fragmentation patterns depending on whether they originated from a particular cell or tissue type or from a tumor or from healthy cells, as discussed elsewhere herein. The sequence-variable target regions may show differences in sequence depending on whether they originated from a tumor or from healthy cells. [0293] Analysis of epigenetic target regions from a hypomethylated partition may be less informative in some applications than analysis of sequence-variable target regions from hypermethylated and hypomethylated partitions and epigenetic target regions from a hypermethylated partition. As such, in methods where sequence-variable target regions and epigenetic target regions are being captured, the latter may be captured to a lesser extent than one or more of the sequence-variable target regions are captured from the hypermethylated and hypomethylated partitions and/or to a lesser extent that epigenetic target regions are captured from a hypermethylated partition. For example, sequence-variable target regions can be captured from a portion of a hypomethylated partition that is not pooled with a hypermethylated partition, and the pool can be prepared with some (e.g., a majority, substantially all, or all) of the DNA from a hypermethylated partition and none or some (e.g., a minority) of the DNA from a hypomethylated partition. Such approaches can reduce or eliminate sequencing of epigenetic target regions from hypomethylated partitions, thereby reducing the amount of sequencing data that suffices for further analysis.

In some embodiments, including a minority of the DNA of a hypomethylated partition in the pool facilitates quantification of one or more epigenetic features (e.g., methylation or other epigenetic feature(s) discussed in detail elsewhere herein), e.g., on a relative basis.

In some embodiments, the pool comprises a minority of the DNA of a hypomethylated partition, e.g., less than about 50% of the DNA of a hypomethylated partition, such as less than or equal to about 45%, 40%, 35%, 30%, 25%, 20%, 15%, 10%, or 5% of the DNA of a hypomethylated partition. In some embodiments, the pool comprises about 5%-25% of the DNA of a hypomethylated partition. In some embodiments, the pool comprises about 10%-20% of the DNA of a hypomethylated partition. In some embodiments, the pool comprises about 10% of the DNA of a hypomethylated partition. In some embodiments, the pool comprises about 15% of the DNA of a hypomethylated partition. In some embodiments, the pool comprises about 20% of the DNA of a hypomethylated partition.

In some embodiments, the pool comprises a portion of a hypermethylated partition, which may be at least about 50% of the DNA of a hypermethylated partition. For example, the pool may comprise at least about 55%, 60%, 65%, 70%, 75%, 80%, 85%, 90%, or 95% of the DNA of a hypermethylated partition. In some embodiments, the pool comprises 50-55%, 55-60%, 60-65%, 65-70%, 70-75%, 75-80%, 80-85%, 85-90%, 90-95%, or 95-100% of the DNA of a hypermethylated partition. In some embodiments, the second pool comprises all or substantially all of the DNA of a hypermethylated partition.

In some embodiments, a first pool comprises substantially all or all of the DNA of a hypomethylated partition (e.g., wherein a second pool does not comprise DNA of a hypomethylated partition. In some embodiments, the second pool does not comprise DNA of a hypomethylated partition (e.g., wherein the first pool comprises substantially all or all of the DNA of a hypomethylated partition).

In some embodiments, a second pool comprises a portion of a hypermethylated partition, which may be any of the values and ranges set forth above with respect to a hypomethylated partition. In some embodiments, the second pool comprises all or substantially all of the DNA of a hypermethylated partition.

In an exemplary embodiment, after partitioning, the partitions separately undergo end repair and ligation to adapters comprising molecular barcodes and are then amplified separately. After the amplification, amplified molecules are enriched (still keeping the partitions separate). Post-enrichment, the enriched DNA are pooled according to any of the embodiments described herein, and then amplified again. After amplification, the molecules are sequenced.

In various embodiments, the methods further comprise sequencing the captured DNA, e.g., to different degrees of sequencing depth for the epigenetic and sequence-variable target region sets, consistent with the discussion above.

### I. Sequencing

In general, sample nucleic acids, including nucleic acids flanked by adapters, with or without prior amplification can be subject to sequencing. Sequencing methods include, for example, Sanger sequencing, high-throughput sequencing, pyrosequencing, sequencing-by synthesis, single-molecule sequencing, nanopore sequencing, semiconductor sequencing, sequencing-by-ligation, sequencing-by-hybridization, Digital Gene Expression (Helicos), Next generation sequencing (NGS), Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, Maxim-Gilbert sequencing, primer walking, and sequencing using PacBio, SOLiD, Ion Torrent, or Nanopore platforms.

In some embodiments, sequencing comprises detecting and/or distinguishing unmodified and modified nucleobases. For example, PacBio sequencing (e.g., single-molecule real-time (SMRT) sequencing) offers the ability to directly detect of, e.g., 5-methylcytosine and 5-hydroxymethylcytosine as well as unmodified cytosine. See, e.g., Schatz., Nature Methods. 14(4): 347-348 (2017); and U.S. Pat. No. 9,150,918. Also, Oxford nanopore sequencing systems (e.g., MinION sequencer) that can directly detect methylation of DNA (for example: 5-methylcytosine and 5-hydroxymethylcytosine) can be used here. Sequencing reactions can be performed in a variety of sample processing units, which may multiple lanes, multiple channels, multiple wells, or other mean of processing multiple sample sets substantially simultaneously. Sample processing unit can also include multiple sample chambers to enable processing of multiple runs simultaneously. Similarly, Ion Torrent sequencing may also be used to directly detect methylation. Thus, in some embodiments, methylation status can be determined during sequencing, e.g., without or independently of a partitioning step or a conversion procedure such as bisulfite treatment.

The sequencing reactions can be performed on one or more forms of nucleic acids, such as those known to contain markers of cancer or of other disease. The sequencing reactions can also be performed on any nucleic acid fragments present in the sample. In some embodiments, sequence coverage of the genome may be less than 5%, 10%, 15%, 20%, 25%, 30%, 40%, 50%, 60%, 70%, 80%, 90%, 95%, 99%, 99.9% or 100%. In some embodiments, the sequence reactions may provide for sequence coverage of at least 5%, 10%, 15%, 20%, 25%, 30%, 40%, 50%, 60%, 70%, or 80% of the genome. Sequence coverage can be performed on at least 5, 10, 20, 70, 100, 200 or 500 different genes, or at most 5000, 2500, 1000, 500 or 100 different genes. [0304] Simultaneous sequencing reactions may be performed using multiplex sequencing. In some cases, cell-free nucleic acids may be sequenced with at least 1000, 2000, 3000, 4000, 5000, 6000, 7000, 8000, 9000, 10000, 50000, 100,000 sequencing reactions. In other cases, cell-free nucleic acids may be sequenced with less than 1000, 2000, 3000, 4000, 5000, 6000, 7000, 8000, 9000, 10000, 50000, 100,000 sequencing reactions. Sequencing reactions may be performed sequentially or simultaneously. Subsequent data analysis may be performed on all or part of the sequencing reactions. In some cases, data analysis may be performed on at least 1000, 2000, 3000, 4000, 5000, 6000, 7000, 8000, 9000, 10000, 50000, 100,000 sequencing reactions. In other cases, data analysis may be performed on less than 1000, 2000, 3000, 4000, 5000, 6000, 7000, 8000, 9000, 10000, 50000, 100,000 sequencing reactions. An exemplary read depth is 1000-50000 reads per locus (base). 1.

### 1. Differential Depth of Sequencing

In some embodiments, nucleic acids corresponding to a sequence-variable target region set are sequenced to a greater depth of sequencing than nucleic acids corresponding to an epigenetic target region set. For example, the depth of sequencing for nucleic acids corresponding to sequence variant target region sets may be at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold greater, or 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, 14- to 15-fold, or 15- to 100-fold greater, than the depth of sequencing for nucleic acids corresponding to an epigenetic target region set. In some embodiments, said depth of sequencing is at least 2-fold greater. In some embodiments, said depth of sequencing is at least 5-fold greater. In some embodiments, said depth of sequencing is at least 10-fold greater. In some embodiments, said depth of sequencing is 4- to 10-fold greater. In some embodiments, said depth of sequencing is 4- to 100-fold greater.

In some embodiments, DNA corresponding to a sequence-variable target region set, and/or to an epigenetic target region set are sequenced concurrently, e.g., in the same sequencing cell (such as the flow cell of an Illumina sequencer) and/or in the same composition, which may be a combined or pooled composition resulting from recombining separately captured sets or a composition obtained by, e.g., capturing the cfDNA corresponding to the sequence-variable target region set, and/or the captured cfDNA corresponding to an epigenetic target region set in the same vessel.

### J. Analysis

In some embodiments, any of the methods disclosed herein comprises determining a likelihood that the subject from which the DNA was obtained has a disease or disorder related to the immune system, such as an infection, transplant rejection, or cancer or precancer.

In some embodiments, any of the methods disclosed herein comprises identifying the presence of DNA produced by a tumor (or neoplastic cells, or cancer cells) or by precancer cells. In some embodiments, a method described herein comprises determining an indication of cancer in the subject. In some such embodiments, determination of the indication of cancer facilitates detection or diagnosis or cancer or precancer, or determination of cancer prognosis or cancer treatment options. For example, determining the metrics from the one or more classification regions and the one or more control regions can help in determining the indication of cancer. In some embodiments, the metrics can be used to determine the tumor fraction of a sample.

The present methods can be used to diagnose presence of conditions, particularly cancer or precancer, in a subject, to characterize conditions (e.g., staging cancer or determining heterogeneity of a cancer), monitor response to treatment of a condition, effect prognosis risk of developing a condition or subsequent course of a condition. The present disclosure can also be useful in determining the efficacy of a particular treatment option. For example, the change in the tumor fraction or determining the methylation status of one or regions can be useful in determining whether the patient is responding to the treatment or not. In another example, perhaps certain treatment options may be correlated with methylation profiles of cancers over time. This correlation may be useful in selecting a therapy.

Additionally, if a cancer is observed to be in remission after treatment, the present methods can be used to monitor residual disease or recurrence of disease.

The types and number of cancers that may be detected may include blood cancers, brain cancers, lung cancers, skin cancers, nose cancers, throat cancers, liver cancers, bone cancers, lymphomas, pancreatic cancers, skin cancers, bowel cancers, rectal cancers, thyroid cancers, bladder cancers, kidney cancers, mouth cancers, stomach cancers, solid state tumors, heterogeneous tumors, homogenous tumors and the like. Type and/or stage of cancer can be detected from genetic variations including mutations, rare mutations, indels, copy number variations, transversions, translocations, recombination, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine.

Genetic data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

Further, the methods of the disclosure may be used to characterize the heterogeneity of an abnormal condition in a subject. Such methods can include, e.g., generating a genetic profile of extracellular polynucleotides derived from the subject, wherein the genetic profile comprises a plurality of data resulting from copy number variation and rare mutation analyses. In some embodiments, an abnormal condition is cancer or precancer. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease. Again, in the example of cancer, there may be multiple tumor foci, perhaps where one or more foci are the result of metastases that have spread from a primary site.

The present methods can be used to generate or profile, fingerprint or set of data that is a summation of genetic information derived from different cells in a heterogeneous disease. This set of data may comprise copy number variation, epigenetic variation, or other mutation analyses alone or in combination.

The present methods can be used to diagnose, prognose, monitor or observe cancers, or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

An exemplary method for determining tumor information through NGS comprises the following steps:
1. Extracting cfDNA from a blood sample
2. Performing one or more nucleobase methylation state detection processes
3. Amplifying the cfDNA via PCR amplification
4. Capturing DNA that correspond to a number of classification regions using target-specific probes.
5. Amplifying the captured DNA and assaying in multiplex on an NGS instrument.
6. Analyzing NGS data using one or more methods disclosed herein to determine the tumor information.

Another exemplary method for determining tumor information through NGS comprises the following steps:
1. Extracting cfDNA from a blood sample.
2. Ligating the cfDNA with adapters comprising molecular barcodes
3. Subjecting the ligated cfDNA to a nucleobase methylation state detection method
4. Capturing DNA comprising classification regions using target-specific probes, wherein the probes are designed such that they can be targeted to capture converted or unconverted molecules.
5. Amplifying the captured DNA and assaying in multiplex on an NGS instrument.
6. Analyzing NGS data using one or more methods disclosed herein to determine the tumor information.

Another exemplary method for determining an indication of cancer or for determining methylation status of a target region (e.g., promoter region) through NGS comprises the following steps:
1. Extracting cfDNA from a blood sample
2. Partitioning cfDNA into a plurality of partitions by contacting the DNA with an agent that recognizes a modified cytosine, such as methyl cytosine, in the DNA
3. Ligating the partitions with adapters comprising molecular barcodes
4. Treating the hyper and/or intermediate partitions with a procedure that affect a first nucleobase in the cfDNA differently from a second nucleobase (e.g., bisulfite method, EM-seq)
5. Amplifying the partitions post-treating the procedure via PCR amplification
6. Capturing DNA comprising hypermethylated target regions, hypomethylated target regions and control regions using target-specific probes.
7. Amplifying the captured DNA and assaying in multiplex on an NGS instrument.
8. Analyzing NGS data using one or more methods disclosed herein to determine whether the target region (e.g., promoter region) is methylated or not.

In some embodiments, instead of using cfDNA from a blood sample, the exemplary methods discussed above can also be used with DNA samples obtained from tissue sample, stool sample or bodily fluids like urine sample. In these embodiments, the DNA can be a whole genomic DNA. In instances, where whole genomic DNA is used, an additional step of fragmenting the DNA (after DNA extraction, but prior to step 2) is performed to the methods discussed above. In some embodiments of methods described herein, molecular barcodes consist of nucleotides that are not altered by a procedure that affects a first nucleobase in the DNA differently from a second nucleobase in the DNA, such as any of those described herein (e.g., mC along with A, T, and G where the procedure is bisulfite conversion or any other conversion that does not affect mC; hmC along with A, T, and G where the procedure is a conversion that does not affect hmC; etc.). In some embodiments of methods described herein, the molecular tags do not comprise nucleotides that are altered by a procedure that affects a first nucleobase in the DNA differently from a second nucleobase in the DNA, such as any of those described herein (e.g., the tags do not comprise unmodified C where the procedure is bisulfite conversion or any other conversion that affects C; the tags do not comprise mC where the procedure is a conversion that affects mC; the tags do not comprise hmC where the procedure is a conversion that affects hmC; etc.).

### Additional Features of Certain Disclosed Methods

### A. Samples

A sample can be any biological sample isolated from a subject. A sample can be a bodily sample. Samples can include body tissues, such as known or suspected solid tumors, whole blood, platelets, serum, plasma, stool, red blood cells, white blood cells or leucocytes, endothelial cells, tissue biopsies, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, urine. Samples are preferably body fluids, particularly blood and fractions thereof, and urine. A sample can be in the form originally isolated from a subject or can have been subjected to further processing to remove or add components, such as cells, or enrich for one component relative to another. Thus, a preferred body fluid for analysis is plasma or serum containing cell-free nucleic acids.

In some embodiments, a population of nucleic acids is obtained from a serum, plasma or blood sample from a subject suspected of having neoplasia, a tumor, precancer, or cancer or previously diagnosed with neoplasia, a tumor, precancer, or cancer. The population includes nucleic acids having varying levels of sequence variation, epigenetic variation, and/or post replication or transcriptional modifications. Post-replication modifications include modifications of cytosine, particularly at the 5-position of the nucleobase, e.g., 5-methylcytosine, 5-hydroxymethylcytosine, 5-formylcytosine and 5-carboxylcytosine.

A sample can be isolated or obtained from a subject and transported to a site of sample analysis. The sample may be preserved and shipped at a desirable temperature, e.g., room temperature, 4° C., −20° C., and/or −80° C. A sample can be isolated or obtained from a subject at the site of the sample analysis. The subject can be a human, a mammal, an animal, a companion animal, a service animal, or a pet. The subject may have a cancer, precancer, infection, transplant rejection, or other disease or disorder related to changes in the immune system. The subject may not have cancer or a detectable cancer symptom. The subject may have been treated with one or more cancer therapy, e.g., any one or more of chemotherapies, antibodies, vaccines or biologies. The subject may be in remission. The subject may or may not be diagnosed of being susceptible to cancer or any cancer-associated genetic mutations/disorders.

In some embodiments, the sample comprises plasma. The volume of plasma obtained can depend on the desired read depth for sequenced regions. Exemplary volumes are 0.4-40 ml, 5-20 ml, 10-20 ml. For examples, the volume can be 0.5 mL, 1 mL, 5 mL 10 mL, 20 mL, 30 mL, or 40 mL. A volume of sampled plasma may be 5 to 20 mL.

A sample can comprise various amount of nucleic acid that contains genome equivalents. For example, a sample of about 30 ng DNA can contain about 10,000 (10⁴) haploid human genome equivalents and, in the case of cfDNA, about 200 billion (2×lOⁿ) individual polynucleotide molecules. Similarly, a sample of about 100 ng of DNA can contain about 30,000 haploid human genome equivalents and, in the case of cfDNA, about 600 billion individual molecules.

A sample can comprise nucleic acids from different sources, e.g., from cells and cell-free of the same subject, from cells and cell-free of different subjects. A sample can comprise nucleic acids carrying mutations. For example, a sample can comprise DNA carrying germline mutations and/or somatic mutations. Germline mutations refer to mutations existing in germline DNA of a subject. Somatic mutations refer to mutations originating in somatic cells of a subject, e.g., precancer cells or cancer cells. A sample can comprise DNA carrying cancer-associated mutations (e.g., cancer-associated somatic mutations). A sample can comprise an epigenetic variant (i.e., a chemical or protein modification), wherein the epigenetic variant associated with the presence of a genetic variant such as a cancer-associated mutation. In some embodiments, the sample comprises an epigenetic variant associated with the presence of a genetic variant, wherein the sample does not comprise the genetic variant.

Exemplary amounts of cell-free nucleic acids in a sample before amplification range from about 1 fg to about 1 pg, e.g., 1 pg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of cell-free nucleic acid molecules. The amount can be at least 1 fg, at least 10 fg, at least 100 fg, at least 1 pg, at least 10 pg, at least 100 pg, at least 1 ng, at least 10 ng, at least 100 ng, at least 150 ng, or at least 200 ng of cell-free nucleic acid molecules. The amount can be up to 1 femtogram (fg), 10 fg, 100 fg, 1 picogram (pg), 10 pg, 100 pg, 1 ng, 10 ng, 100 ng, 150 ng, or 200 ng of cell-free nucleic acid molecules. The method can comprise obtaining 1 femtogram (fg) to 200 ng-[0326] Cell-free nucleic acids are nucleic acids not contained within or otherwise bound to a cell or in other words nucleic acids remaining in a sample after removing intact cells. Cell-free nucleic acids include DNA, RNA, and hybrids thereof, including genomic DNA, mitochondrial DNA, siRNA, miRNA, circulating RNA (cRNA), tRNA, rRNA, small nucleolar RNA (snoRNA), Piwi-interacting RNA (piRNA), long non-coding RNA (long ncRNA), or fragments of any of these. Cell-free nucleic acids can be double-stranded, single-stranded, or a hybrid thereof. A cell-free nucleic acid can be released into bodily fluid through secretion or cell death processes, e.g., cellular necrosis and apoptosis. Some cell-free nucleic acids are released into bodily fluid from cancer cells e.g., circulating tumor DNA, (ctDNA). Others are released from healthy cells. In some embodiments, cfDNA is cell-free fetal DNA (cffDNA) In some embodiments, cell free nucleic acids are produced by tumor cells. In some embodiments, cell free nucleic acids are produced by a mixture of tumor cells and non-tumor cells.

Cell-free nucleic acids have an exemplary size distribution of about 100-500 nucleotides, with molecules of 110 to about 230 nucleotides representing about 90% of molecules, with a mode of about 168 nucleotides and a second minor peak in a range between 240 to 440 nucleotides.

Cell-free nucleic acids can be isolated from bodily fluids through a fractionation step in which cell-free nucleic acids, as found in solution, are separated from intact cells and other non-soluble components of the bodily fluid. Partitioning may include techniques such as centrifugation or filtration. Alternatively, cells in bodily fluids can be lysed and cell-free and cellular nucleic acids processed together. Generally, after addition of buffers and wash steps, nucleic acids can be precipitated with an alcohol. Further clean up steps may be used such as silica-based columns to remove contaminants or salts. Non-specific bulk carrier nucleic acids, such as C 1 DNA, DNA or protein for bisulfite sequencing, hybridization, and/or ligation, may be added throughout the reaction to optimize certain aspects of the procedure such as yield. [0329] After such processing, samples can include various forms of nucleic acid including double stranded DNA, single stranded DNA, and single stranded RNA. In some embodiments, single stranded DNA and RNA can be converted to double stranded forms so they are included in subsequent processing and analysis steps. [0330] DNA molecules can be linked to adapters at either one end or both ends. Typically, double-stranded molecules are blunt ended by treatment with a polymerase with a 5′-3′ polymerase and a 3′-5′ exonuclease (or proof-reading function), in the presence of all four standard nucleotides. Klenow large fragment and T4 polymerase are examples of suitable polymerase. The blunt ended DNA molecules can be ligated with at least partially double stranded adapter (e.g., a Y shaped or bell-shaped adapter). Alternatively, complementary nucleotides can be added to blunt ends of sample nucleic acids and adapters to facilitate ligation. Contemplated herein are both blunt end ligation and sticky end ligation. In blunt end ligation, both the nucleic acid molecules and the adapter tags have blunt ends. In sticky-end ligation, typically, the nucleic acid molecules bear an “A” overhang and the adapters bear a “T” overhang.

### B. Tags

Tags comprising barcodes can be incorporated into or otherwise joined to adapters. Tags can be incorporated by ligation, overlap extension PCR among other methods.

### i) Molecular Tagging Strategies

Molecular tagging refers to a tagging practice that allows one to differentiate among DNA molecules from which sequence reads originated. Tagging strategies can be divided into unique tagging and non-unique tagging strategies. In unique tagging, all or substantially all of the molecules in a sample bear a different tag, so that reads can be assigned to original molecules based on tag information alone. Tags used in such methods are sometimes referred to as “unique tags”. In non-unique tagging, different molecules in the same sample can bear the same tag, so that other information in addition to tag information is used to assign a sequence read to an original molecule. Such information may include start and stop coordinate, coordinate to which the molecule maps, start or stop coordinate alone, etc. Tags used in such methods are sometimes referred to as “non-unique tags”. Accordingly, it is not necessary to uniquely tag every molecule in a sample. It suffices to uniquely tag molecules falling within an identifiable class within a sample. Thus, molecules in different identifiable families can bear the same tag without loss of information about the identity of the tagged molecule.

In certain embodiments of non-unique tagging, the number of different tags used can be sufficient that there is a very high likelihood (e.g., at least 99%, at least 99.9%, at least 99.99% or at least 99.999% that all DNA molecules of a particular group bear a different tag. It is to be noted that when barcodes are used as tags, and when barcodes are attached, e.g., randomly, to both ends of a molecule, the combination of barcodes, together, can constitute a tag. This number, in term, is a function of the number of molecules falling into the calls. For example, the class may be all molecules mapping to the same start-stop position on a reference genome. The class may be all molecules mapping across a particular genetic locus, e.g., a particular base or a particular region (e.g., up to 100 bases or a gene or an exon of a gene). In certain embodiments, the number of different tags used to uniquely identify a number of molecules, z, in a class can be between any of 2*z, 3*z, 4*z, 5*z, 6*z, 7*z, 8*z, 9*z, 10*z, 11*z, 12*z, 13*z, 14*z, 15*z, 16*z, 17*z, 18*z, 19*z, 20*z or 100*z (e.g., lower limit) and any of 100,000*z, 10,000*z, 1000*z or 100*z (e.g., upper limit).

For example, in a sample of about 5 ng to 30 ng of cell free DNA, one expects around 3000 molecules to map to a particular nucleotide coordinate, and between about 3 and 10 molecules having any start coordinate to share the same stop coordinate. Accordingly, about 50 to about 50,000 different tags (e.g., between about 6 and 220 barcode combinations) can suffice to uniquely tag all such molecules. To uniquely tag all 3000 molecules mapping across a nucleotide coordinate, about 1 million to about 20 million different tags would be required. [0336] Generally, assignment of unique or non-unique tags barcodes in reactions follows methods and systems described by US patent applications 20010053519, 20030152490, 20110160078, and U.S. Pat. Nos. 6,582,908 and 7,537,898 and 9,598,731. Tags can be linked to sample nucleic acids randomly or non-randomly. [0337] The unique tags may be loaded so that more than about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the unique tags may be loaded so that less than about 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the average number of unique tags loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags per genome sample.

A preferred format uses 20-50 different tags (e.g., barcodes) ligated to both ends of target nucleic acids. For example, 35 different tags (e.g., barcodes) ligated to both ends of target molecules creating 35×35 permutations, which equals 1225 for 35 tags. Such numbers of tags are sufficient so that different molecules having the same start and stop points have a high probability (e.g., at least 94%, 99.5%, 99.99%, 99.999%) of receiving different combinations of tags. Other barcode combinations include any number between 10 and 500, e.g., about 15×15, about 35×35, about 75×75, about 100×100, about 250×250, about 500×500.

In some cases, unique tags may be predetermined or random or semi-random sequence oligonucleotides. In other cases, a plurality of barcodes may be used such that barcodes are not necessarily unique to one another in the plurality. In this example, barcodes may be ligated to individual molecules such that the combination of the barcode and the sequence it may be ligated to creates a unique sequence that may be individually tracked. As described herein, detection of non-unique barcodes in combination with sequence data of beginning (start) and end (stop) portions of sequence reads may allow assignment of a unique identity to a particular molecule. The length or number of base pairs, of an individual sequence read may also be used to assign a unique identity to such a molecule. As described herein, fragments from a single strand of nucleic acid having been assigned a unique identity, may thereby permit subsequent identification of fragments from the parent strand.

### C. Amplification

Sample nucleic acids flanked by adapters can be amplified by PCR and other amplification methods. Amplification is typically primed by primers that anneal or bind to primer binding sites in adapters flanking a DNA molecule to be amplified. Amplification methods can involve cycles of denaturation, annealing and extension, resulting from thermocycling or can be isothermal as in transcription-mediated amplification. Other amplification methods include the ligase chain reaction, strand displacement amplification, nucleic acid sequence-based amplification, and self-sustained sequence-based replication.

In some embodiments, the present methods perform dsDNA ligations with T-tailed and C-tailed adapters, which result in amplification of at least 50, 60, 70 or 80% of double stranded nucleic acids before linking to adapters. Preferably the present methods increase the amount or number of amplified molecules relative to control methods performed with T-tailed adapters alone by at least 10, 15 or 20%.

### D. Capture Moieties

As discussed above, nucleic acids in a sample can be subject to a capture step, in which molecules having target regions are captured for subsequent analysis. Target capture can involve use of probes (e.g., oligonucleotides) labeled with a capture moiety, such as biotin, and a second moiety or binding partner that binds to the capture moiety, such as streptavidin. In some embodiments, a capture moiety and binding partner can have higher and lower capture yields for different sets of target regions, such as those of the sequence-variable target region set and the epigenetic target region set, respectively, as discussed elsewhere herein. Methods comprising capture moieties are further described in, for example, U.S. Pat. No. 9,850,523, issuing Dec. 26, 2017, which is incorporated herein by reference.

Capture moieties include, without limitation, biotin, avidin, streptavidin, a nucleic acid comprising a particular nucleotide sequence, a hapten recognized by an antibody, and magnetically attractable particles. The extraction moiety can be a member of a binding pair, such as biotin/streptavidin or hapten/antibody. In some embodiments, a capture moiety that is attached to an analyte is captured by its binding pair which is attached to an isolatable moiety, such as a magnetically attractable particle or a large particle that can be sedimented through centrifugation. The capture moiety can be any type of molecule that allows affinity separation of nucleic acids bearing the capture moiety from nucleic acids lacking the capture moiety. Exemplary capture moieties are biotin which allows affinity separation by binding to streptavidin linked or linkable to a solid phase or an oligonucleotide, which allows affinity separation through binding to a complementary oligonucleotide linked or linkable to a solid phase.

### E. Collections of Target-Specific Probes

In some embodiments, a collection of target-specific probes is used in a method comprising an epigenetic target region set and/or a sequence-variable target region set, as described herein. In some embodiments, the collection of target-specific probes comprises target binding probes specific for a sequence-variable target region set and target-binding probes specific for an epigenetic target region set. In some embodiments, the capture yield of the target binding probes specific for the sequence-variable target region set is higher (e.g., at least 2-fold higher) than the capture yield of the target-binding probes specific for the epigenetic target region set. In some embodiments, the collection of target-specific probes is configured to have a capture yield specific for the sequence-variable target region set higher (e.g., at least 2-fold higher) than its capture yield specific for the epigenetic target region set.

In some embodiments, the capture yield of the target-binding probes specific for the sequence-variable target region set is at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than the capture yield of the target-binding probes specific for the epigenetic target region set. In some embodiments, the capture yield of the target-binding probes specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than the capture yield of the target-binding probes specific for the epigenetic target region set.

In some embodiments, the collection of target-specific probes is configured to have a capture yield specific for the sequence-variable target region set at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than its capture yield for the epigenetic target region set. In some embodiments, the collection of target-specific probes is configured to have a capture yield specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than its capture yield specific for the epigenetic target region set.

The collection of probes can be configured to provide higher capture yields for the sequence-variable target region set in various ways, including concentration, different lengths and/or chemistries (e.g., that affect affinity), and combinations thereof. Affinity can be modulated by adjusting probe length and/or including nucleotide modifications as discussed below.

In some embodiments, the target-specific probes specific for the sequence-variable target region set are present at a higher concentration than the target-specific probes specific for the epigenetic target region set. In some embodiments, concentration of the target-binding probes specific for the sequence-variable target region set is at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than the concentration of the target-binding probes specific for the epigenetic target region set. In some embodiments, the concentration of the target-binding probes specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than the concentration of the target-binding probes specific for the epigenetic target region set. In such embodiments, concentration may refer to the average mass per volume concentration of individual probes in each set.

In some embodiments, the target-specific probes specific for the sequence-variable target region set have a higher affinity for their targets than the target-specific probes specific for the epigenetic target region set. Affinity can be modulated in any way known to those skilled in the art, including by using different probe chemistries. For example, certain nucleotide modifications, such as cytosine 5-methylation (in certain sequence contexts), modifications that provide a heteroatom at the T sugar position, and LNA nucleotides, can increase stability of double-stranded nucleic acids, indicating that oligonucleotides with such modifications have relatively higher affinity for their complementary sequences. See, e.g., Severin et ah, Nucleic Acids Res. 39: 8740-8751 (2011); Freier et ah, Nucleic Acids Res. 25: 4429-4443 (1997); U.S. Pat. No. 9,738,894. Also, longer sequence lengths will generally provide increased affinity. Other nucleotide modifications, such as the substitution of the nucleobase hypoxanthine for guanine, reduce affinity by reducing the amount of hydrogen bonding between the oligonucleotide and its complementary sequence. In some embodiments, the target-specific probes specific for the sequence-variable target region set have modifications that increase their affinity for their targets. In some embodiments, alternatively or additionally, the target-specific probes specific for the epigenetic target region set have modifications that decrease their affinity for their targets. In some embodiments, the target-specific probes specific for the sequence-variable target region set have longer average lengths and/or higher average melting temperatures than the target-specific probes specific for the epigenetic target region set. These embodiments may be combined with each other and/or with differences in concentration as discussed above to achieve a desired fold difference in capture yield, such as any fold difference or range thereof described above.

In some embodiments, the target-specific probes comprise a capture moiety. The capture moiety may be any of the capture moieties described herein, e.g., biotin. In some embodiments, the target-specific probes are linked to a solid support, e.g., covalently or non-covalently such as through the interaction of a binding pair of capture moieties. In some embodiments, the solid support is a bead, such as a magnetic bead.

In some embodiments, the target-specific probes specific for the sequence-variable target region set and/or the target-specific probes specific for the epigenetic target region set comprise a capture moiety as discussed above, e.g., probes comprising capture moieties and sequences selected to tile across a panel of regions, such as genes.

In some embodiments, the target-specific probes are provided in a single composition.

The single composition may be a solution (liquid or frozen). Alternatively, it may be a lyophilizate.

Alternatively, the target-specific probes may be provided as a plurality of compositions, e.g., comprising a first composition comprising probes specific for the epigenetic target region set and a second composition comprising probes specific for the sequence-variable target region set. These probes may be mixed in appropriate proportions to provide a combined probe composition with any of the foregoing fold differences in concentration and/or capture yield. Alternatively, they may be used in separate capture procedures (e.g., with aliquots of a sample or sequentially with the same sample) to provide first and second compositions comprising captured epigenetic target regions and sequence-variable target regions, respectively.

### i) Probes Specific for Epigenetic Target Regions

The probes for the epigenetic target region set may comprise probes specific for one or more types of target regions likely to differentiate DNA originating from different types of immune cells, including rare immune cell types, and/or to differentiate DNA from precancerous or neoplastic (e.g., tumor or cancer) cells from healthy cells, e.g., non-neoplastic circulating cells. Exemplary types of such regions are discussed in detail herein. The probes for the epigenetic target region set may also comprise probes for one or more control regions, e.g., as described herein.

In some embodiments, the probes for the epigenetic target region probe set have a footprint of at least 100 kb, e.g., at least 200 kb, at least 300 kb, or at least 400 kb. In some embodiments, the probes for the epigenetic target region set have a footprint in the range of 100-1000 kb, e.g., 100-200 kb, 200-300 kb, 300-400 kb, 400-500 kb, 500-600 kb, 600-700 kb, 700-800 kb, 800-900 kb, and 900-1,000 kb. In some embodiments, the probes for the epigenetic target region probe set have a footprint of at least 5 kb, e.g., at least 10, 20, or 50 kb. a. Hypermethylation target regions.

In some embodiments, for the methods using methylation-sensitive conversion (e.g., bisulfite or EM-seq), the probes can be designed to target either the converted molecules or unconverted molecules depending on the type of methylation-sensitive conversion and the target region being enriched. For example, if bisulfite treatment is used, the unmethylated cytosines in the DNA molecules will be converted to dihydrouracil and methylated cytosines will remain unconverted as cytosine. For capturing DNA molecules in the hypermethylated target regions (where the molecules of interest to cancer or any other disease under investigation will be hypermethylated), the probes can be designed to capture the unconverted molecules, whereas for capturing molecules in the hypomethylated target regions (where the molecules of interest to cancer or any other disease under investigation will be hypomethylated or unmethylated), the probes can be designed to capture the converted molecules.

In some embodiments, the probes for the epigenetic target region set comprise probes specific for one or more hypermethylation target regions. The hypermethylation target regions may be any of those set forth above. For example, in some embodiments, the probes specific for hypermethylation target regions comprise probes specific for a plurality of loci that are differentially methylated in different immune cell types. In some embodiments, each immune cell type specific hypermethylation target region comprises at least one CpG site that is methylated with a frequency greater than or equal to 0.3, 0.4, 0.5, or 0.6 in one immune cell type and with a frequency less than or equal to 0.1, 0.2, or 0.3 in all other immune cell types. In some embodiments, each immune cell type specific hypermethylation target region comprises at least two CpG sites within 100 base pairs of each other that are each methylated with a frequency greater than or equal to 0.3, 0.4, 0.5, or 0.6 in one immune cell type and with a frequency less than or equal to 0.1, 0.2, or 0.3 in all other immune cell types. In some such embodiments, each immune cell type specific hypermethylation target region comprises a total of at least 2, 3, 4, 5, 6, 7, 8, 9, or 10 CpG sites within 150 base pairs or within 200 base pairs, wherein fewer than three of the at least 2, 3, 4, 5, 6, 7, 8, 9, or 10 CpG sites are methylated with a frequency greater than 0.1, 0.2, or 0.3 in any normal tissue type. In some embodiments, each immune cell type specific epigenetic target region set comprises at least 3, at least 5, at least 10, at least 20, or at least 30 hypermethylation target regions that are uniquely hypermethylated in each one of the immune cell types that are identified in the method.

In some embodiments, the probes specific for hypermethylation target regions comprise probes specific for a plurality of loci listed in Table 1, e.g., at least 10%, 20%, 30%, 40%, 50%, 60%, 70%, 80%, 90%, or 100% of the loci listed in Table 1. In some embodiments, the probes specific for hypermethylation target regions comprise probes specific for a plurality of loci listed in Table 2, e.g., at least 10%, 20%, 30%, 40%, 50%, 60%, 70%, 80%, 90%, or 100% of the loci listed in Table 2. In some embodiments, the probes specific for hypermethylation target regions comprise probes specific for a plurality of loci listed in Table 1 or Table 2, e.g., at least 10%, 20%, 30%, 40%, 50%, 60%, 70%, 80%, 90%, or 100% of the loci listed in Table 1 or Table 2.

In some embodiments, for each locus included as a target region, there may be one or more probes with a hybridization site that binds between the transcription start site and the stop codon (the last stop codon for genes that are alternatively spliced) of the gene. In some embodiments, the one or more probes bind within 300 bp of the listed position, e.g., within 200 or 100 bp. In some embodiments, a probe has a hybridization site overlapping the position listed above. In some embodiments, the probes specific for the hypermethylation target regions include probes specific for one, two, three, four, or five subsets of hypermethylation target regions that collectively show hypermethylation in one, two, three, four, or five of breast, colon, kidney, liver, and lung cancers. b. Hypomethylation target regions.

In some embodiments, the probes for the epigenetic target region set comprise probes specific for one or more hypomethylation target regions. The hypomethylation target regions may be any of those set forth above. For example, in some embodiments, the probes specific for hypomethylation target regions comprise probes specific for a plurality of loci that are differentially methylated in different immune cell types. In some embodiments, each immune cell type specific hypomethylation target region comprises at least one CpG site that is methylated with a frequency less than or equal to 0.1, 0.2, or 0.3 in one immune cell type and with a frequency greater than or equal to 0.3, 0.4, 0.5, or 0.6 in all other immune cell types. In some embodiments, each immune cell type specific hypomethylation target region comprises at least two CpG sites within 100 base pairs of each other that are each methylated with a frequency less than or equal to 0.1, 0.2, or 0.3 in one immune cell type and with a frequency greater than or equal to 0.3, 0.4, 0.5, or 0.6 in all other immune cell types. In some such embodiments, each immune cell type specific hypomethylation target region comprises a total of at least 2, 3, 4, 5, 6, 7, 8, 9, or 10 CpG sites within 150 base pairs or within 200 base pairs, wherein fewer than three of the at least 2, 3, 4, 5, 6, 7, 8, 9, or 10 CpG sites are methylated with a frequency less than 0.1, 0.2, or 0.3 in any normal tissue type. In some embodiments, each immune cell type specific epigenetic target region set comprises at least 3, at least 5, at least 10, at least 20, or at least 30 hypomethylation target regions that are uniquely hypomethylated in each one of the immune cell types that are identified in the method.

In some embodiments, the probes specific for one or more hypomethylation target regions may include probes for regions such as repeated elements, e.g., LINE1 elements, Alu elements, centromeric tandem repeats, pericentromeric tandem repeats, and satellite DNA, and intergenic regions that are ordinarily methylated in healthy cells may show reduced methylation in tumor cells.

In some embodiments, probes specific for hypomethylation target regions include probes specific for repeated elements and/or intergenic regions. In some embodiments, probes specific for repeated elements include probes specific for one, two, three, four, or five of LINE1 elements, Alu elements, centromeric tandem repeats, pericentromeric tandem repeats, and/or satellite DNA.

Exemplary probes specific for genomic regions that show cancer-associated hypomethylation include probes specific for nucleotides 8403565-8953708 and/or 151104701-151106035 of human chromosome 1. In some embodiments, the probes specific for hypomethylation target regions include probes specific for regions overlapping or comprising nucleotides 8403565-8953708 and/or 151104701-151106035 of human chromosome 1.

In some embodiments, the probes for the epigenetic target region set include probes specific for CTCF binding regions. In some embodiments, the probes specific for CTCF binding regions comprise probes specific for at least 10, 20, 50, 100, 200, or 500 CTCF binding regions, or 10-20, 20-50, 50-100, 100-200, 200-500, or 500-1000 CTCF binding regions, e.g., such as CTCF binding regions described above or in one or more of CTCFBSDB or the Cuddapah et al., Martin et al., or Rhee et al. articles cited above. In some embodiments, the probes for the epigenetic target region set comprise at least 100 bp, at least 200 bp at least 300 bp, at least 400 bp, at least 500 bp, at least 750 bp, or at least 1000 bp upstream and downstream regions of the CTCF binding sites. d. Transcription start sites.

In some embodiments, the probes for the epigenetic target region set include probes specific for transcriptional start sites. In some embodiments, the probes specific for transcriptional start sites comprise probes specific for at least 10, 20, 50, 100, 200, or 500 transcriptional start sites, or 10-20, 20-50, 50-100, 100-200, 200-500, or 500-1000 transcriptional start sites, e.g., such as transcriptional start sites listed in DBTSS. In some embodiments, the probes for the epigenetic target region set comprise probes for sequences at least 100 bp, at least 200 bp, at least 300 bp, at least 400 bp, at least 500 bp, at least 750 bp, or at least 1000 bp upstream and downstream of the transcriptional start sites.

As noted above, although focal amplifications are somatic mutations, they can be detected by sequencing based on read frequency in a manner analogous to approaches for detecting certain epigenetic changes such as changes in methylation. As such, regions that may show focal amplifications in cancer can be included in the epigenetic target region set, as discussed above. In some embodiments, the probes specific for the epigenetic target region set include probes specific for focal amplifications. In some embodiments, the probes specific for focal amplifications include probes specific for one or more of AR, BRAF, CCND1, CCND2, CCNE1, CDK4, CDK6, EGFR, ERBB2, FGFR1, FGFR2, KIT, KRAS, MET, MYC, PDGFRA, PIK3CA, and RAFI. For example, in some embodiments, the probes specific for focal amplifications include probes specific for one or more of at least 2, 3, 4, 5, 6, 7, 8, 9, 10, 11, 12, 13, 14, 15, 16, 17, or 18 of the foregoing targets.

### ii) Control Regions

In some embodiments, the probes specific for the epigenetic target region set include probes specific for positive control regions that are expected to be methylated in essentially all samples. In some embodiments, the probes specific for the epigenetic target region set include probes specific for negative control regions that are expected to be hypomethylated or unmethylated in essentially all samples.

iii) Probes Specific for Sequence-Variable Target Regions

The probes for the sequence-variable target region set may comprise probes specific for a plurality of regions known to undergo somatic mutations in cancer. The probes may be specific for any sequence-variable target region set described herein. Exemplary sequence-variable target region sets are discussed in detail herein, e.g., in the sections above concerning captured sets. [0366] In some embodiments, the sequence-variable target region probe set has a footprint of at least 0.5 kb, e.g., at least 1 kb, at least 2 kb, at least 5 kb, at least 10 kb, at least 20 kb, at least 30 kb, or at least 40 kb. In some embodiments, the epigenetic target region probe set has a footprint in the range of 0.5-100 kb, e.g., 0.5-2 kb, 2-10 kb, 10-20 kb, 20-30 kb, 30-40 kb, 40-50 kb, 50-60 kb, 60-70 kb, 70-80 kb, 80-90 kb, and 90-100 kb.

In some embodiments, probes specific for the sequence-variable target region set comprise probes specific for at least a portion of at least 5, at least 10, at least 15, at least 20, at least 25, at least 30, at least 35, at least 40, at least 45, at least 50, at least 55, at least 60, at least 65, or at 70 of the genes of Table 4. In some embodiments, probes specific for the sequence-variable target region set comprise probes specific for the at least 5, at least 10, at least 15, at least 20, at least 25, at least 30, at least 35, at least 40, at least 45, at least 50, at least 55, at least 60, at least 65, or 70 of the SNVs of Table 3. In some embodiments, probes specific for the sequence-variable target region set comprise probes specific for at least 1, at least 2, at least 3, at least 4, at least 5, or 6 of the fusions of Table 3. In some embodiments, probes specific for the sequence-variable target region set comprise probes specific for at least a portion of at least 1, at least 2, or 3 of the indels of Table 4. In some embodiments, probes specific for the sequence-variable target region set comprise probes specific for at least a portion of at least 5, at least 10, at least 15, at least 20, at least 25, at least 30, at least 35, at least 40, at least 45, at least 50, at least 55, at least 60, at least 65, at least 70, or 73 of the genes of Table 5. In some embodiments, probes specific for the sequence-variable target region set comprise probes specific for at least 5, at least 10, at least 15, at least 20, at least 25, at least 30, at least 35, at least 40, at least 45, at least 50, at least 55, at least 60, at least 65, at least 70, or 73 of the SNVs of Table 5. In some embodiments, probes specific for the sequence-variable target region set comprise probes specific for at least 1, at least 2, at least 3, at least 4, at least 5, or 6 of the fusions of Table 5. In some embodiments, probes specific for the sequence-variable target region set comprise probes specific for at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the indels of Table 5. In some embodiments, probes specific for the sequence-variable target region set comprise probes specific for at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 of the genes of Table 6.

In some embodiments, the probes specific for the sequence-variable target region set comprise probes specific for target regions from at least 10, 20, 30, or 35 cancer-related genes, such as AKTI, ALK, BRAF, CCND1, CDK2A, CTNNB1, EGFR, ERBB2, ESR1, FGFR1, FGFR2, FGFR3, FOXL2, GAT A3, GNA11, GNAQ, GNAS, HRAS, IDH1, IDH2, KIT, KRAS, MED 12, MET, MYC, NFE2L2, NRAS, PDGFRA, PIK3CA, PPP2R1A, PTEN, RET, STK11, TP53, and U2AF 1.

### Precision Treatments

The precision diagnostics provided by the improved computer system 324 may result in precision treatment plans, which may be identified by the computer system 324 (and/or curated by health professionals). For example, one type of precision diagnostic and treatment may relate to genes in the homologous recombination repair (HRR) pathway.

Homologous recombination is a type of genetic recombination in which nucleotide sequences are exchanged between two similar or identical molecules of DNA. It is most widely used by cells to accurately repair harmful breaks that occur on both strands of DNA, known as double-strand breaks (DSB). HRR provides a mechanism for the error-free removal of damage present in DNA that has replicated (S and G2 phases), to eliminate chromosomal breaks before the cell division occurs. The primary model for how homologous recombination repairs double-strand breaks in DNA is homologous recombination repair pathway which mediates the double-strand break repair (DSBR) pathway and the synthesis-dependent strand annealing (SDSA) pathway. Germline and somatic deficiencies in homologous recombination genes have been strongly linked to breast, ovarian and prostate cancers.

The number and types of variant nucleotides in a sample can provide an indication of the amenability of the subject providing the sample to treatment, i.e., therapeutic intervention. For example, various poly ADP ribose polymerase (PARP) inhibitors have been shown to stop the growth of tumors from breast, ovarian and prostate cancers caused by hereditary mutations in the BRCA1 or BRCA2 genes. Some of these therapeutic agents may inhibit base excision repair (BER), which may compensate for the deficiency of HRR.

On the other hand, certain BRCA and HRR wildtype patients may not achieve clinical benefit from treatment with a PARP inhibitor. Furthermore, not all ovarian cancer patients with a BRCA mutation will respond to a PARP inhibitor. Moreover, different types of mutations may indicate different therapies. For example, somatic heterozygous deletions in HRR genes may indicate a different therapy than somatic homozygous deletions. Thus, the state of genetic material may influence therapy. In one example, a PARP inhibitor may be administered to an individual harboring a somatic homozygous deletion in a HRR gene, but not to an individual harboring a wildtype allele or somatic heterozygous deletions in the HRR gene.

In some implementations, a subject having HRD as determined by any of the methods disclosed may be administered a targeted therapy. The targeted therapy may comprise a PARP inhibitor. Examples of PARP inhibitors that may be administered include one or more of: VELIPARIB, OLAPARIB, TALAZOPARIB, RUCAPARIB, NIRAPARIB, PAMIPARIB, CEP 9722 (Cephalon), E7016 (Eisai), E7449 (Eisai, a PARP 1/2 and tankyrase 1/2 inhibitor), or 3-Aminobenzamide. In some implementations, the targeted therapy may comprise at least one base excision repair (BER) inhibitor. For example, OLAPARIB may inhibit BER. In certain implementations, the targeted therapy may comprise combination of a PARP inhibitor and radiotherapy. In an implementation, the combination of a PARP inhibitor and radiotherapy would permit the PARP inhibitor to lead to formation of double strand breaks from the single-strand breaks generated by the radiotherapy in tumor tissue (e.g., tissue with BRCA1/BRCA2 mutations). This combination can provide more powerful therapy per radiation dose.

### Related Therapies

In certain embodiments, the methods disclosed herein relate to identifying and administering therapies, such as customized therapies, to patients or subjects based on the determination of the presence or absence or levels of epigenomic and/or genetic variation. In some embodiments, the patient or subject has a given disease, disorder or condition, e.g., any of the cancers or other conditions described elsewhere herein. Essentially any cancer therapy (e.g., surgical therapy, radiation therapy, chemotherapy, immunotherapy, and/or the like) may be included as part of these methods.

Typically, the disease under consideration is a type of cancer. Non-limiting examples of such cancers include biliary tract cancer, bladder cancer, transitional cell carcinoma, urothelial carcinoma, brain cancer, gliomas, astrocytomas, breast cancer, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas, Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma.

Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (scid), sickle cell disease, spinal muscular atrophy, Tay-Sachs, thalassemia, trimethylaminuria, Turner syndrome, velocardiofacial syndrome, WAGR syndrome, Wilson disease, or the like.

In certain embodiments, the therapies can include one or more of treatments for target therapies, including abemaciclib (Verzenio), abiraterone acetate (Zytiga), acalabrutinib (Calquence), adagrasib (Krazati), ado-trastuzumab emtansine (Kadcyla), afatinib dimaleate (Gilotrif), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), atezolizumab (Tecentriq), avapritinib (Ayvakit), avelumab (Bavencio), axicabtagene ciloleucel (Yescarta), axitinib (Inlyta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib-s-malate (Cabometyx), cabozantinib-s-malate (Cometriq), capmatinib hydrochloride (Tabrecta), carfilzomib (Kyprolis), cemiplimab-rwlc (Libtayo), ceritinib (Zykadia), cetuximab (Erbitux), ciltacabtagene autoleucel (Carvykti), cobimetinib fumarate (Cotellic), copanlisib hydrochloride (Aliqopa), crizotinib (Xalkori), dabrafenib (Tafmlar), dabrafenib mesylate (Tafmlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elacestrant dihydrochloride (Orserdu), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib hydrochloride (Tarceva), everolimus (Afinitor), exemestane (Aromasin), fam-trastuzumab deruxtecan-nxki (Enhertu), fam-trastuzumab deruxtecan-nxki (Enhertu), fedratinib hydrochloride (Inrebic), fulvestrant (Faslodex), futibatinib (Lytgobi), gefitinib (Iressa), gemtuzumab ozogamicin (Mylotarg), gilteritinib fumarate (Xospata), glasdegib maleate (Daurismo), ibritumomab tiuxetan (Zevalin), ibrutinib (Imbruvica), idecabtagene vicleucel (Abecma), idelalisib (Zydelig), imatinib mesylate (Gleevec), infigratinib phosphate (Truseltiq), inotuzumab ozogamicin (Besponsa), iobenguane 1 131 (Azedra), ipilimumab (Yervoy), isatuximab-irfc (Sarclisa), ivosidenib (Tibsovo), ixazomib citrate (Ninlaro), Ianreotide acetate (SomatulineDepot), lapatinib ditosylate (Tykerb), larotrectinib sulfate (Vitrakvi), lenvatinib mesylate (Lenvima), letrozole (Femara), lisocabtagene maraleucel (Breyanzi), loncastuximab tesirine-Ipyl (Zynlonta), lorlatinib (Lorbrena), lutetium Lu 177 vipivotide tetraxetan (Pluvicto), lutetium Lu 177-dotatate (Lutathera), margetuximab-cmkb (Margenza), midostaurin (Rydapt), mirvetuximab soravtansine-gynx (Elahere), mobocertinib succinate (Exkivity), mogamulizumab-kpkc (Poteligeo), mosunetuzumab-axgb (Lunsumio), moxetumomab pasudotox-tdfk(Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), nivolumab and relatlimab-rmbw (Opdualag), obinutuzumab (Gazyva), ofatumumab (Arzerra), olaparib (Lynparza), olutasidenib (Rezlidhia), osimertinib mesylate (Tagrisso), pacritinib citrate (Vonjo), palbociclib (Ibrance), panitumumab (Vectibix), pazopanib hydrochloride(Votrient), pembrolizumab (Keytruda), pemigatinib(Pemazyre), pertuzumab (Perjeta), pertuzumab, trastuzumab, and hyaluronidase-zzxf (Phesgo), pexidartinib hydrochloride (Turalio), pirtobrutinib (Jaypirca), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), retifanlimab-dlwr (Zynyz), ribociclib (Kisqali), ripretinib (Qinlock), rituximab (Rituxan), rituximab and hyaluronidase human (Rituxan Hycela), romidepsin (Istodax), rucaparib camsylate(Rubraca), ruxolitinib phosphate (Jakafi), sacituzumab govitecan-hziy (Trodelvy), selinexor (Xpovio), selpercatinib (Retevmo), selumetinib sulfate (Koselugo), siltuximab (Sylvant), sirolimus protein-bound particles (Fyarro), sonidegib (Odomzo), sorafenib tosylate (Nexavar), sotorasib (Lumakras), sunitinib malate (Sutent), tafasitamab-cxix (Monjuvi), tagraxofusp-erzs (Elzonris), talazoparib tosylate (Talzenna), tamoxifen citrate (Soltamox), tazemetostat hydrobromide (Tazverik), tebentafusp-tebn (Kimmtrak), teclistamab-cqyv (Tecvayli), temsirolimus (Torisel), tepotinib hydrochloride (Tepmetko), tisagenlecleucel (Kymriah), tisotumab vedotin-tftv (Tivdak), tivozanib hydrochloride (Fotivda), toremifene (Fareston), trametinib (Mekinist), trametinib dimethyl sulfoxide (Mekinist), trastuzumab (Herceptin), tremelimumab-actl (Imjudo), tretinoin (Vesanoid), tucatinib (Tukysa), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap).

In certain embodiments, the therapy administered to a subject comprises at least one chemotherapy drug. In some embodiments, the chemotherapy drug may comprise alkylating agents (for example, but not limited to, Chlorambucil, Cyclophosphamide, Cisplatin and Carboplatin), nitrosoureas (for example, but not limited to, Carmustine and Lomustine), anti-metabolites (for example, but not limited to, Fluorauracil, Methotrexate and Fludarabine), plant alkaloids and natural products (for example, but not limited to, Vincristine, Paclitaxel and Topotecan), anti-tumor antibiotics (for example, but not limited to, Bleomycin, Doxorubicin and Mitoxantrone), hormonal agents (for example, but not limited to, Prednisone, Dexamethasone, Tamoxifen and Leuprolide) and biological response modifiers (for example, but not limited to, Herceptin and Avastin, Erbitux and Rituxan). In some embodiments, the chemotherapy administered to a subject may comprise FOLFOX or FOLFIRI. In certain embodiments, a therapy may be administered to a subject that comprises at least one PARP inhibitor. In certain embodiments, the PARP inhibitor may include OLAPARIB, TALAZOPARIB, RUCAPARIB, NIRAPARIB (trade name ZEJULA), among others. In some embodiments, the methods comprise administering a therapy comprising a PARP inhibitor, such as olaparib, to a subject determined to have homologous recombination repair (HRR) gene or deficiency (HRD), such as with BRCA1, BRCA2, ATM, BARD1, BRIP1, CDK12, CHEK1, CHEK2, FANCL, PALB2, RAD51B, RAD51C, RAD51D, and RAD54L alterations. In some embodiments, the subject has a metastatic castrate resistant prostate cancer (mCRPC). In some embodiments, the PARP inhibitor, such as olaprib is used to treat a subject having ovarian cancer, breast cancer, pancreatic cancer, or mCRPC, wherein the subject is determined to have alterations in BRCA1, BRCA2, and/or ATM.

In some embodiments, essentially any cancer therapy (e.g., surgical therapy, radiation therapy, chemotherapy, immunotherapy, and/or the like) may be included as part of these methods. Customized therapies can include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, the immunotherapy or immunotherapeutic agent targets an immune checkpoint molecule. Certain tumors are able to evade the immune system by co-opting an immune checkpoint pathway. Thus, targeting immune checkpoints has emerged as an effective approach for countering a tumor's ability to evade the immune system and activating anti-tumor immunity against certain cancers. Pardoll, Nature Reviews Cancer, 2012, 12:252-264.

In certain embodiments, the immune checkpoint molecule is an inhibitory molecule that reduces a signal involved in the T cell response to antigen. For example, CTLA4 is expressed on T cells and plays a role in downregulating T cell activation by binding to CD80 (aka B7.1) or CD86 (aka B7.2) on antigen presenting cells. PD-1 is another inhibitory checkpoint molecule that is expressed on T cells. PD-1 limits the activity of T cells in peripheral tissues during an inflammatory response. In addition, the ligand for PD-1 (PD-L1 or PD-L2) is commonly upregulated on the surface of many different tumors, resulting in the downregulation of anti-tumor immune responses in the tumor microenvironment. In certain embodiments, the inhibitory immune checkpoint molecule is CTLA4 or PD-1. In other embodiments, the inhibitory immune checkpoint molecule is a ligand for PD-1, such as PD-L1 or PD-L2. In other embodiments, the inhibitory immune checkpoint molecule is a ligand for CTLA4, such as CD80 or CD86. In other embodiments, the inhibitory immune checkpoint molecule is lymphocyte activation gene 3 (LAG3), killer cell immunoglobulin like receptor (KIR), T cell membrane protein 3 (TIM3), galectin 9 (GAL9), or adenosine A2a receptor (A2aR).

Antagonists that target these immune checkpoint molecules can be used to enhance antigen-specific T cell responses against certain cancers. Accordingly, in certain embodiments, the immunotherapy or immunotherapeutic agent is an antagonist of an inhibitory immune checkpoint molecule. In certain embodiments, the inhibitory immune checkpoint molecule is PD-1. In certain embodiments, the inhibitory immune checkpoint molecule is PD-L1. In certain embodiments, the antagonist of the inhibitory immune checkpoint molecule is an antibody (e.g., a monoclonal antibody). In certain embodiments, the antibody or monoclonal antibody is an anti-CTLA4, anti-PD-1, anti-PD-L1, or anti-PD-L2 antibody. In certain embodiments, the antibody is a monoclonal anti-PD-1 antibody. In some embodiments, the antibody is a monoclonal anti-PD-L1 antibody. In certain embodiments, the monoclonal antibody is a combination of an anti-CTLA4 antibody and an anti-PD-1 antibody, an anti-CTLA4 antibody and an anti-PD-L1 antibody, or an anti-PD-L1 antibody and an anti-PD-1 antibody. In certain embodiments, the anti-PD-1 antibody is one or more of pembrolizumab (Keytruda®) or nivolumab (Opdivo®). In certain embodiments, the anti-CTLA4 antibody is ipilimumab (Yervoy®). In certain embodiments, the anti-PD-L1 antibody is one or more of atezolizumab (Tecentriq®), avelumab (Bavencio®), or durvalumab (Imfinzi®). In certain embodiments, immunotherapy, such as pembrolizumab, is used to treat a subject determined to have a high microsatellite instability status (MSI-H). In certain embodiments, the immunotherapy, such as pembrolizumab, is used to treat a subject determined to have a high tumor mutational burden (TMB), for example, then the TMB status is greater than or equal to 10 mutations per megabase. In certain embodiment, the immunotherapy, such as pembrolizumab, is used to treat a subject determined to a have a mismatch repair deficiency (dMMR), such as in genes comprising MLH1, PMS2, MSH2 and MSH6.

In certain embodiments, the immunotherapy or immunotherapeutic agent is an antagonist (e.g., antibody) against CD80, CD86, LAG3, KIR, TIM3, GAL9, or A2aR. In other embodiments, the antagonist is a soluble version of the inhibitory immune checkpoint molecule, such as a soluble fusion protein comprising the extracellular domain of the inhibitory immune checkpoint molecule and an Fc domain of an antibody. In certain embodiments, the soluble fusion protein comprises the extracellular domain of CTLA4, PD-1, PD-L1, or PD-L2. In some embodiments, the soluble fusion protein comprises the extracellular domain of CD80, CD86, LAG3, KIR, TIM3, GAL9, or A2aR. In one embodiment, the soluble fusion protein comprises the extracellular domain of PD-L2 or LAG3.

In certain embodiments, the immune checkpoint molecule is a co-stimulatory molecule that amplifies a signal involved in a T cell response to an antigen. For example, CD28 is a co-stimulatory receptor expressed on T cells. When a T cell binds to antigen through its T cell receptor, CD28 binds to CD80 (aka B7.1) or CD86 (aka B7.2) on antigen-presenting cells to amplify T cell receptor signaling and promote T cell activation. Because CD28 binds to the same ligands (CD80 and CD86) as CTLA4, CTLA4 is able to counteract or regulate the co-stimulatory signaling mediated by CD28. In certain embodiments, the immune checkpoint molecule is a co-stimulatory molecule selected from CD28, inducible T cell co-stimulator (ICOS), CD137, OX40, or CD27. In other embodiments, the immune checkpoint molecule is a ligand of a co-stimulatory molecule, including, for example, CD80, CD86, B7RP1, B7-H3, B7-H4, CD137L, OX40L, or CD70.

Agonists that target these co-stimulatory checkpoint molecules can be used to enhance antigen-specific T cell responses against certain cancers. Accordingly, in certain embodiments, the immunotherapy or immunotherapeutic agent is an agonist of a co-stimulatory checkpoint molecule. In certain embodiments, the agonist of the co-stimulatory checkpoint molecule is an agonist antibody and preferably is a monoclonal antibody. In certain embodiments, the agonist antibody or monoclonal antibody is an anti-CD28 antibody. In other embodiments, the agonist antibody or monoclonal antibody is an anti-ICOS, anti-CD137, anti-OX40, or anti-CD27 antibody. In other embodiments, the agonist antibody or monoclonal antibody is an anti-CD80, anti-CD86, anti-B7RP1, anti-B7-H3, anti-B7-H4, anti-CD137L, anti-OX40L, or anti-CD70 antibody.

In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the subject and/or patients who are receiving, or who have received, the same therapy as the subject. A customized or targeted therapy (or therapies) may be identified when the nucleic variant and the comparator results satisfy certain classification criteria (e.g., are a substantial or an approximate match).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by any method known in the art, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

In certain embodiments, the present methods are also useful in determining the efficacy of particular treatment options. For example, the number of variations detected, irrespective of their precise identity, is a predictor of amenability to immunotherapy because the mutations create neoepitopes that can be subject of immune attack (see e.g., US20200370129).

Other variations or copy number variations indicate suitability of a particular drug. Some examples of such variations are as follows:

TABLE 1
List of cancer types with associated biomarker target and drug

| Cancer type | Drug | Target |
|---|---|---|
| Breast | abemaciclib | CDK4, CDK6 |
| Prostate | abiraterone acetate | CYP17A1 |
| Leukemia | acalabrutinib | BTK |
| Lymphoma | acalabrutinib | BTK |
| Lung | adagrasib | KRAS G12C |
| Breast | ado-trastuzumab | Tubulin, Her2 |
| | emtansine | |
| Lung | afatinib dimaleate | EGFR + EGFR exon 21 L858R |
| | | mutation + EGFR-Ex19del + HER2 + |
| | | HER4 |
| Lung | alectinib | ALK, RET |
| Leukemia | alemtuzumab | CD52 |
| Skin | alitretinoin | RARα + RARβ2 + RARγ |
| Soft tissue sarcoma | alitretinoin | RARα + RARβ2 + RARγ |
| Breast | alpelisib | PI3Kα |
| Lung | amivantamab-vmjw | EGFR + c-MET |
| Breast | anastrozole | Aromatase |
| Prostate | apalutamide | AR |
| Leukemia | asciminib | Bcr-Abl |
| | hydrochloride | |
| Bladder | atezolizumab | PDL1 |
| Liver and bile duct | atezolizumab | PDL1 |
| Lung | atezolizumab | PDL1 |
| Skin | atezolizumab | PDL1 |
| Soft tissue sarcoma | atezolizumab | PDL1 |
| Gastrointestinal | avapritinib | PDGFRα + c-Kit |
| Systemic | avapritinib | PDGFRα + c-Kit |
| mastocytosis | | |
| Bladder | avelumab | PDL1 |
| Endocrine and | avelumab | PDL1 |
| neuroendocrine | | |
| tumors | | |
| Kidney | avelumab | PDL1 |
| Skin | Avelumab | PDL1 |
| Lymphoma | axicabtagene | CD19 |
| | ciloleucel | |
| Kidney | axitinib | VEGFR1 + VEGFR2 + VEGFR3 |
| Lymphoma | belinostat | HDAC |
| Brain | belzutifan | HIF-2A |
| Kidney | belzutifan | HIF-2A |
| Pancreatic | belzutifan | HIF-2A |
| Ovarian epithelial, | bevacizumab | VEGF-A |
| fallopian tube, and | | |
| primary peritoneal | | |
| Brain | bevacizumab | VEGF-A |
| Cervical | bevacizumab | VEGF-A |
| Colorectal | bevacizumab | VEGF-A |
| Kidney | bevacizumab | VEGF-A |
| Liver and bile duct | bevacizumab | VEGF-A |
| Lung | bevacizumab | VEGF-A |
| Lymphoma | bexarotene | RXRs |
| Skin | binimetinib | MEK1, MEK2 |
| Leukemia | blinatumomab | CD19, CD3 |
| Lymphoma | Bortezomib | Proteasome |
| Multiple myeloma | bortezomib | Proteasome |
| Leukemia | bosutinib | BCR-ABL, SRC |
| Lymphoma | brentuximab vedotin | CD130, Tubulin |
| Leukemia | brexucabtagene | CD19 |
| | autoleucel | |
| Lymphoma | brexucabtagene | CD19 |
| | autoleucel | |
| Lung | brigatinib | ALK, EGFR, FLT3, IGF-1R, ROS1 |
| Prostate | cabazitaxel | Tubulin |
| Kidney | cabozantinib-s- | AXL + RET + ROS1 + TYRO3 + Tie-2 + |
| | malate | TrkB + VEGFR1 + VEGFR2 + VEGFR3 + |
| | | c-Kit + c-Met |
| Liver and bile duct | cabozantinib-s- | AXL + RET + ROS1 + TYRO3 + Tie-2 + |
| | malate | TrkB + VEGFR1 + VEGFR2 + VEGFR3 + |
| | | c-Kit + c-Met |
| Thyroid | cabozantinib-s- | AXL + RET + ROS1 + TYRO3 + Tie-2 + |
| | malate | TrkB + VEGFR1 + VEGFR2 + VEGFR3 + |
| | | c-Kit + c-Met |
| Lung | capmatinib | c-Met |
| | hydrochloride | |
| Multiple myeloma | carfilzomib | Proteasome |
| Lung | cemiplimab-rwlc | PD-1 |
| Skin | cemiplimab-rwlc | PD-1 |
| Lung | ceritinib | ALK + IGF-1R + INSR + ROS1 |
| Colorectal | cetuximab | EGFR |
| Multiple myeloma | ciltacabtagene | BCMA |
| | autoleucel | |
| Skin | cobimetinib fumarate | MEK1, MEK2 |
| Lymphoma | copanlisib | PI3Kα, PI3Kδ |
| | hydrochloride | |
| Lung | crizotinib | MTH1 |
| Lymphoma | crizotinib | MTH1 |
| Myofibroblastic | crizotinib | MTH1 |
| Brain | dabrafenib | BRAF, CRAF |
| Lung | dabrafenib mesylate | BRAF, CRAF |
| Skin | dabrafenib mesylate | BRAF, CRAF |
| Solid tumors | dabrafenib mesylate | BRAF, CRAF |
| anywhere in the body | | |
| Thyroid | dabrafenib mesylate | BRAF, CRAF |
| Lung | dacomitinib | EGFR + EGFR exon 21 L858R |
| | | mutation + EGFR-Ex19del + HER2 + |
| | | HER4 |
| Multiple myeloma | daratumumab | CD38 + Hyaluronic acid |
| Multiple myeloma | daratumumab and | CD38 + Hyaluronic acid |
| | hyaluronidase-fihj | |
| Prostate | darolutamide | AR |
| Leukemia | dasatinib | Bcr-Abl + EphA2 + FYN + LCK + |
| | | PDGFRβ + Protein-tyrosine kinases + |
| | | SRC + YES1 + c-Kit |
| Neuroblastoma | dinutuximab | GD2 |
| Endometrial | dostarlimab-gxly | PD-1 |
| Solid tumors | dostarlimab-gxly | PD-1 |
| anywhere in the body | | |
| Liver and bile duct | durvalumab | PDL1 |
| Lung | durvalumab | PDL1 |
| Leukemia | duvelisib | PI3Kγ + PI3Kδ |
| Breast | elacestrant | Erα/ESR1 |
| | dihydrochloride | |
| Multiple myeloma | elotuzumab | SLAMF7 |
| Leukemia | enasidenib mesylate | IDH2 |
| Colorectal | encorafenib | BRAF, BRAF V600E |
| Skin | encorafenib | BRAF V600E, V600K mutation |
| Bladder | enfortumab vedotin- | Nectin-4, Tubulins |
| | ejfv | |
| Lung | entrectinib | ALK + ROS1 + TRKA + TrkB + TrkC |
| Solid tumors | entrectinib | ALK + ROS1 + TRKA + TrkB + TrkC |
| anywhere in the body | | |
| Prostate | enzalutamide | AR |
| Bladder | erdafitinib | FGFR1, FGFR2, FGFR3, FGFR4 |
| Lung | erlotinib | EGFR antagonists, EGFR exon 21 |
| | hydrochloride | L858R mutation inhibitors, EGFR- |
| | | Ex19del inhibitors |
| Pancreatic | erlotinib | EGFR + EGFR exon 21 L858R |
| | hydrochloride | mutation + EGFR-Ex19del |
| Breast | everolimus | mTORC1, mTORC2 |
| Kidney | everolimus | mTORC1, mTORC2 |
| Pancreatic | everolimus | mTORC1, mTORC2 |
| Breast | exemestane | Aromatase |
| Breast | fam-trastuzumab | HER2, TOP1 |
| | deruxtecan-nxki | |
| Gastric | fam-trastuzumab | HER2, TOP1 |
| | deruxtecan-nxki | |
| Lung | fam-trastuzumab | HER2, TOP1 |
| | deruxtecan-nxki | |
| Myelodysplastic and | fedratinib | JAK2, FLT3 |
| myeloproliferative | hydrochloride | |
| disorders | | |
| Breast | fulvestrant | ER |
| Liver and bile duct | futibatinib | FGFRs |
| Lung | gefitinib | EGFR + EGFR exon 21 L858R |
| | | mutation + EGFR-Exon 19 deletion |
| Leukemia | gemtuzumab | CD33, DNA |
| | ozogamicin | |
| Leukemia | gilteritinib fumarate | AXL + FLT3 |
| Leukemia | glasdegib maleate | SMO |
| Lymphoma | ibritumomab tiuxetan | CD20 |
| Leukemia | ibrutinib | BTK |
| Lymphoma | ibrutinib | BTK |
| Multiple myeloma | idecabtagene | BCMA |
| | vicleucel | |
| Leukemia | idelalisib | PI3Kδ |
| Dermatofibrosarcoma | imatinib mesylate | BCR-ABL, PDGFR, C-Kit |
| protuberans | | |
| Gastrointestinal | imatinib mesylate | BCR-ABL, PDGFR, C-Kit |
| Leukemia | imatinib mesylate | BCR-ABL, PDGFR, C-Kit |
| Myelodysplastic and | imatinib mesylate | BCR-ABL, PDGFR, C-Kit |
| myeloproliferative | | |
| disorders | | |
| Systemic | imatinib mesylate | BCR-ABL, PDGFR, C-Kit |
| mastocytosis | | |
| Liver and bile duct | infigratinib phosphate | FGFR1 + FGFR2 + FGFR3 + FGFR4 |
| Leukemia | inotuzumab | CD22 + DNA |
| | ozogamicin | |
| Endocrine and | iobenguane\|131 | NET |
| neuroendocrine | | |
| tumors | | |
| Colorectal | ipilimumab | CTLA4 |
| Esophageal | ipilimumab | CTLA4 |
| Kidney | ipilimumab | CTLA4 |
| Liver and bile duct | ipilimumab | CTLA4 |
| Lung | ipilimumab | CTLA4 |
| Malignant | ipilimumab | CTLA4 |
| mesothelioma | | |
| Skin | ipilimumab | CTLA4 |
| Multiple myeloma | isatuximab-irfc | CD38 |
| Leukemia | ivosidenib | IDH1 |
| Multiple myeloma | ixazomib citrate | proteasome |
| Endocrine and | lanreotide acetate | SSTR |
| neuroendocrine | | |
| tumors | | |
| Breast | lapatinib ditosylate | EGFR |
| Solid tumors | larotrectinib sulfate | TRKA + TrkB + TrkC |
| anywhere in the body | | |
| Lung | Lazertinib | EGFR, EGFR exon 19 deletion, EGFR |
| | | exon 21 L858R substitution |
| Endometrial | lenvatinib mesylate | FGFR1 + FGFR2 + FGFR3 + FGFR4 + |
| | | PDGFRα + RET + VEGFR1 + VEGFR2 + |
| | | VEGFR3 + c-Kit |
| Kidney | lenvatinib mesylate | FGFR1 + FGFR2 + FGFR3 + FGFR4 + |
| | | PDGFRα + RET + VEGFR1 + VEGFR2 + |
| | | VEGFR3 + c-Kit |
| Liver and bile duct | lenvatinib mesylate | FGFR1 + FGFR2 + FGFR3 + FGFR4 + |
| | | PDGFRα + RET + VEGFR1 + VEGFR2 + |
| | | VEGFR3 + c-Kit |
| Thyroid | lenvatinib mesylate | FGFR1 + FGFR2 + FGFR3 + FGFR4 + |
| | | PDGFRα + RET + VEGFR1 + VEGFR2 + |
| | | VEGFR3 + c-Kit |
| Breast | letrozole/Ribociclib | CDK4/ER |
| | Succinate | |
| Lymphoma | lisocabtagene | CD19 |
| | maraleucel | |
| Lymphoma | loncastuximab | CD19 + DNA |
| | tesirine-lpyl | |
| Lung | lorlatinib | ALK + ROS1 |
| Prostate | lutetium Lu 177 | PSMA |
| | vipivotide tetraxetan | |
| Endocrine and | lutetium Lu 177- | SSTR2 |
| neuroendocrine | dotatate | |
| tumors | | |
| Breast | margetuximab-cmkb | HER2 |
| Leukemia | midostaurin | FLT3 + PDGFR + PKC + Syk + |
| | | VEGFR2 + c-Kit |
| Systemic | midostaurin | FLT3 + PDGFR + PKC + Syk + |
| mastocytosis | | VEGFR2 + c-Kit |
| Ovarian epithelial, | mirvetuximab | FOLR1 + Tubulin |
| fallopian tube, and | soravtansine-gynx | |
| primary peritoneal | | |
| Lung | mobocertinib | EGFR exon 20 + HER2 exon 20 |
| | succinate | |
| Lymphoma | mogamulizumab- | CCR4 |
| | kpkc | |
| Lymphoma | mosunetuzumab- | CD20, CD3 |
| | axgb | |
| Leukemia | moxetumomab | CD22 |
| | pasudotox-tdfk | |
| Neuroblastoma | naxitamab-gqgk | GD2 |
| Lung | necitumumab | EGFR |
| Breast | neratinib maleate | EGFR, HER2, HER4 |
| Leukemia | nilotinib | Bcr-Abl + CSF-1R + DDR1 + PDGFR + |
| | | c-Kit |
| Ovarian epithelial, | niraparib tosylate | PARP1 + PARP2 |
| fallopian tube, and | monohydrate | |
| primary peritoneal | | |
| Bladder | nivolumab | PD-1 |
| Colorectal | nivolumab | PD-1 |
| Esophageal | nivolumab | PD-1 |
| Kidney | nivolumab | PD-1 |
| Liver and bile duct | nivolumab | PD-1 |
| Lung | nivolumab | PD-1 |
| Lymphoma | nivolumab | PD-1 |
| Malignant | nivolumab | PD-1 |
| mesothelioma | | |
| Skin | nivolumab | PD-1 |
| Stomach (gastric) | nivolumab | PD-1 |
| Skin | nivolumab and | LAG3 + PD-1 |
| | relatlimab-rmbw | |
| Leukemia | obinutuzumab | CD20 |
| Lymphoma | obinutuzumab | CD20 |
| Leukemia | ofatumumab | CD20 |
| Ovarian epithelial, | olaparib | PARP1, PARP2, PARP3 |
| fallopian tube, and | | |
| primary peritoneal | | |
| Breast | olaparib | PARP1, PARP2, PARP3 |
| Pancreatic | olaparib | PARP1, PARP2, PARP3 |
| Prostate | olaparib | PARP1, PARP2, PARP3 |
| Leukemia | olutasidenib | IDH1 |
| Lung | osimertinib mesylate | EGFR + EGFR T790M + EGFR exon |
| | | 21 L858R mutation + EGFR-Ex19del |
| Myelodysplastic and | pacritinib citrate | CSF-1R + FLT3 + IRAK1 + JAK2 |
| myeloproliferative | | |
| disorders | | |
| Breast | palbociclib | CDK4, CDK6 |
| Colorectal | panitumumab | EGFR |
| Kidney | pazopanib | FGFR1 + FGFR3 + FIt3L + ITK + LCK + |
| | hydrochloride | PDGFRa + PDGFRB + VEGFR1 + |
| | | VEGFR2 + VEGFR3 + c-Kit |
| Soft tissue sarcoma | pazopanib | FGFR1 + FGFR3 + FIt3L + ITK + LCK + |
| | hydrochloride | PDGFRa + PDGFRB + VEGFR1 + |
| | | VEGFR2 + VEGFR3 + c-Kit |
| Breast | pembrolizumab | PD-1 |
| Cervical | pembrolizumab | PD-1 |
| Colorectal | pembrolizumab | PD-1 |
| Endometrial | pembrolizumab | PD-1 |
| Esophageal | pembrolizumab | PD-1 |
| Kidney | pembrolizumab | PD-1 |
| Liver and bile duct | pembrolizumab | PD-1 |
| Lung | pembrolizumab | PD-1 |
| Lymphoma | pembrolizumab | PD-1 |
| Skin | pembrolizumab | PD-1 |
| Solid tumors | pembrolizumab | PD-1 |
| anywhere in the body | | |
| Stomach (gastric) | pembrolizumab | PD-1 |
| Leukemia | pemigatinib | FGFR1, FGFR2, FGFR3, FGFR4 |
| Liver and bile duct | pemigatinib | FGFR1, FGFR2, FGFR3, FGFR4 |
| Lymphoma | pemigatinib | FGFR1, FGFR2, FGFR3, FGFR4 |
| Myelodysplastic and | pemigatinib | FGFR1, FGFR2, FGFR3, FGFR4 |
| myeloproliferative | | |
| disorders | | |
| Breast | pertuzumab | HER2 |
| Breast | pertuzumab, | HER2, Hyaluronic acid |
| | trastuzumab, and | |
| | hyaluronidase-zzxf | |
| Giant cell tumor | pexidartinib | CSF-1R + FLT3 + c-Kit |
| | hydrochloride | |
| Lymphoma | pirtobrutinib | BTK C481S |
| Lymphoma | polatuzumab vedotin- | CD79B + Tubulin |
| | piiq | |
| Leukemia | ponatinib | Bcr-Abl + Protein-tyrosine kinases |
| | hydrochloride | |
| Lymphoma | pralatrexate | Bcr-Abl + Protein-tyrosine kinases |
| Lung | pralsetinib | RET |
| Thyroid | pralsetinib | RET |
| Prostate | radium 223 dichloride | DNA |
| Colorectal | ramucirumab | VEGFR2 |
| Gastric | ramucirumab | VEGFR2 |
| Liver and bile duct | ramucirumab | VEGFR2 |
| Lung | ramucirumab | VEGFR2 |
| Stomach (gastric) | ramucirumab | VEGFR2 |
| Colorectal | regorafenib | Abl family, BRAF, BRAF V600E, CRAF, |
| | | CSF-1R, DDR2, EphA2, FGFR1, |
| | | FGFR2, FRK, MAPK11, PDGFRa, |
| | | PDGFRb, RET, TRKA, Tie-2, VEGFR1, |
| | | VEGFR2, VEGFR3, c-kit |
| Gastrointestinal | regorafenib | Abl family, BRAF, BRAF V600E, CRAF, |
| | | CSF-1R, DDR2, EphA2, FGFR1, |
| | | FGFR2, FRK, MAPK11, PDGFRa, |
| | | PDGFRb, RET, TRKA, Tie-2, VEGFR1, |
| | | VEGFR2, VEGFR3, c-kit |
| Liver and bile duct | regorafenib | Abl family, BRAF, BRAF V600E, CRAF, |
| | | CSF-1R, DDR2, EphA2, FGFR1, |
| | | FGFR2, FRK, MAPK11, PDGFRa, |
| | | PDGFRb, RET, TRKA, Tie-2, VEGFR1, |
| | | VEGFR2, VEGFR3, c-kit |
| Skin | retifanlimab-dlwr | PD-1 |
| Breast | ribociclib | CDK4, CDK6 |
| Gastrointestinal | ripretinib | EGFR + PDGFRa + c-Kit |
| Leukemia | rituximab | CD20 + Hyaluronic acid |
| Lymphoma | rituximab | CD20 + Hyaluronic acid |
| Leukemia | rituximab and | CD20 + Hyaluronic acid |
| | hyaluronidase human | |
| Lymphoma | rituximab and | CD20 + Hyaluronic acid |
| | hyaluronidase human | |
| Lymphoma | romidepsin | HDAC |
| Ovarian epithelial, | rucaparib camsylate | PARP1 + PARP2 + PARP3 |
| fallopian tube, and | | |
| primary peritoneal | | |
| Prostate | rucaparib camsylate | PARP1 + PARP2 + PARP3 |
| Myelodysplastic and | ruxolitinib phosphate | JAK1 + JAK2 |
| myeloproliferative | | |
| disorders | | |
| Breast | sacituzumab | TOP1 |
| | govitecan-hziy | |
| Lymphoma | selinexor | XPO1 |
| Multiple myeloma | selinexor | XPO1 |
| Lung | selpercatinib | RET |
| Solid tumors | selpercatinib | RET |
| anywhere in the body | | |
| Thyroid | selpercatinib | RET |
| Plexiform | selumetinib sulfate | MEK1 + MEK2 |
| neurofibroma | | |
| Soft tissue sarcoma | sirolimus protein- | MUT + mTOR |
| | bound particles | |
| Skin | sonidegib | SMO |
| Kidney | sorafenib tosylate | BRAF inhibitors, CRAF inhibitors, |
| | | FLT3 + PDGFRβ + RET + VEGFR1 + |
| | | VEGFR2 + VEGFR3 + c-Kit |
| Liver and bile duct | sorafenib tosylate | BRAF + CRAF + FLT3 + PDGFRβ + |
| | | RET + VEGFR1 + VEGFR2 + VEGFR3 + |
| | | c-Kit |
| Thyroid | sorafenib tosylate | BRAF + CRAF + FLT3 + PDGFRB + |
| | | RET + VEGFR1 + VEGFR2 + VEGFR3 + |
| | | c-Kit |
| Lung | sotorasib | KRAS G12C |
| Gastrointestinal | sunitinib malate | PDGFR + RTK + VEGFR |
| Kidney | sunitinib malate | PDGFR + RTK + VEGFR |
| Pancreatic | sunitinib malate | PDGFR + RTK + VEGFR |
| Lymphoma | tafasitamab-cxix | CD19 |
| Leukemia | tagraxofusp-erzs | CD123 |
| Breast | talazoparib tosylate | PARP1, PARP2 |
| Breast | tamoxifen citrate | ER |
| Lymphoma | tazemetostat | EZH2 |
| | hydrobromide | |
| Soft tissue sarcoma | tazemetostat | EZH2 |
| | hydrobromide | |
| Skin | tebentafusp-tebn | CD3 + gp100 |
| Multiple myeloma | teclistamab-cqyv | CD3 + gp100 |
| Kidney | temsirolimus | CD3 + gp100 |
| Lung | tepotinib | MET |
| | hydrochloride | |
| Leukemia | tisagenlecleucel | CD19 |
| Lymphoma | tisagenlecleucel | CD19 |
| Cervical | tisotumab vedotin-tftv | Tubulin, tissue factor |
| Kidney | tivozanib | (VEGFR)-1, VEGFR-2 and VEGFR-3, |
| | hydrochloride | c-kit, and PDGFR β |
| Breast | toremifene | ER |
| Brain | trametinib | MEK1 and MEK2 |
| Lung | trametinib dimethyl | MEK1 and MEK2 |
| | sulfoxide | |
| Skin | trametinib dimethyl | MEK1 and MEK2 |
| | sulfoxide | |
| Solid tumors | trametinib dimethyl | MEK1 and MEK2 |
| anywhere in the body | sulfoxide | |
| Thyroid | trametinib dimethyl | MEK1 and MEK2 |
| | sulfoxide | |
| Breast | trastuzumab | HER2 |
| Esophageal | trastuzumab | HER2 |
| Stomach (gastric) | trastuzumab | HER2 |
| Liver and bile duct | tremelimumab-actl | CTLA-4, CD80, and CD86 |
| Lung | tremelimumab-actl | CTLA-4, CD80, and CD86 |
| Breast | tucatinib | HER2 |
| Colorectal | tucatinib | HER2 |
| Thyroid | vandetanib | VEGFR-2, EGFR, RET |
| Skin | vemurafenib | BRAF, BRAF V600E, CRAF, ARAF, |
| | | SRMS, ACK1, MAP4K5, FGR |
| Leukemia | venetoclax | BCL-2 |
| Lymphoma | venetoclax | BCL-2 |
| Lymphoma | vorinostat | HDAC1, HDAC2 and HDAC3 (Class I) |
| | | and HDAC6 (Class II) |
| Leukemia | zanubrutinib | BTK |
| Lymphoma | zanubrutinib | BTK |
| Colorectal | ziv-aflibercept | PGF, VEGF-A |

In certain embodiments, the therapy comprises administrating a treatment to a subject determined to have a copy number amplification. In some embodiments, the treatment may comprise trastuzumab, ado-trastuzumab emtansine, or pertuzumab where the subject was determined to have an ERBB2 (HER2) gene amplification. In some embodiments, the subject has breast cancer or gastric cancer.

In some embodiments, the therapy comprises administering one or more drugs to the subject. For example, patients with non-small lung cancer determined to have either an EGFR exon 19 deletion or an EGFR exon 21 L858R alteration may be treated with amivantamab in combination with lazertinib.

The present methods can be used to generate or profile, fingerprint or set of data that is a summation of genetic information derived from different cells in a heterogeneous disease. This set of data may comprise copy number variation, nucleotide variation, epigenomic information, and/or tumor fraction. In some embodiments, the methods disclosed herein are used to monitor the efficacy or responsiveness of a treatment to the subject. In some embodiments, the methods disclosed herein can be used to determine whether the subject is a candidate for a therapy to treat the cancer or disease.

The present methods can be used to diagnose, prognose, monitor or observe cancers or other diseases of fetal origin. That is, these methodologies can be employed in a pregnant subject to diagnose, prognose, monitor or observe cancers or other diseases in an unborn subject whose DNA and other nucleic acids may co-circulate with maternal molecules.

In certain embodiments, the present methods can be used to determine minimal residual disease (MRD) of a subject, for example, based on a tumor fraction determination. In some embodiments, the methods may be directed to determining MRD by using a tissue-informed assay (i.e., using a tissue sample collected from a patient to determine a personalized panel to enrich for one or more genomic and/or epigenomic variants in a subsequent blood sample from the patient) or a tissue-naive assay.

In certain embodiments, the present methods can integrate genomic and/or epigenomic data with proteomic (proteins and their post-translational modifications), transcriptomic, fragmentomic, immunological, histological, and/or other analyte-specific data to determine disease initiation, progression, malignant transformation, and therapeutic outcomes.

The disclosed methods can be combined with analysis of one or more additional biomarkers. In some embodiments, the disclosed methods are combined with one or more methods, such as but not limited to, methods for assessing gene expression levels, DNA mutations (such as somatic mutations), nucleic acid fragmentation patterns, RNA, non-coding RNA (such as micro RNAs (miRNAs), ribosomal RNAs, transfer RNAs, small nucleolar RNAs (snow RNAs), and/or small nuclear RNAs (snRNAs)) levels, and/or levels of cell types (e.g. immune cell types) and the distribution of cell types, cellular locations, protein levels and/or structural modifications of one or more proteins (such as in a sample from a subject). In some embodiments, the disclosed methods are combined with one or more analyses of genetic variations including mutations, rare mutations, indels, rearrangements, copy number variations, transversions, translocations, recombinations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and/or abnormal changes in nucleic acid 5-methylcytosine.

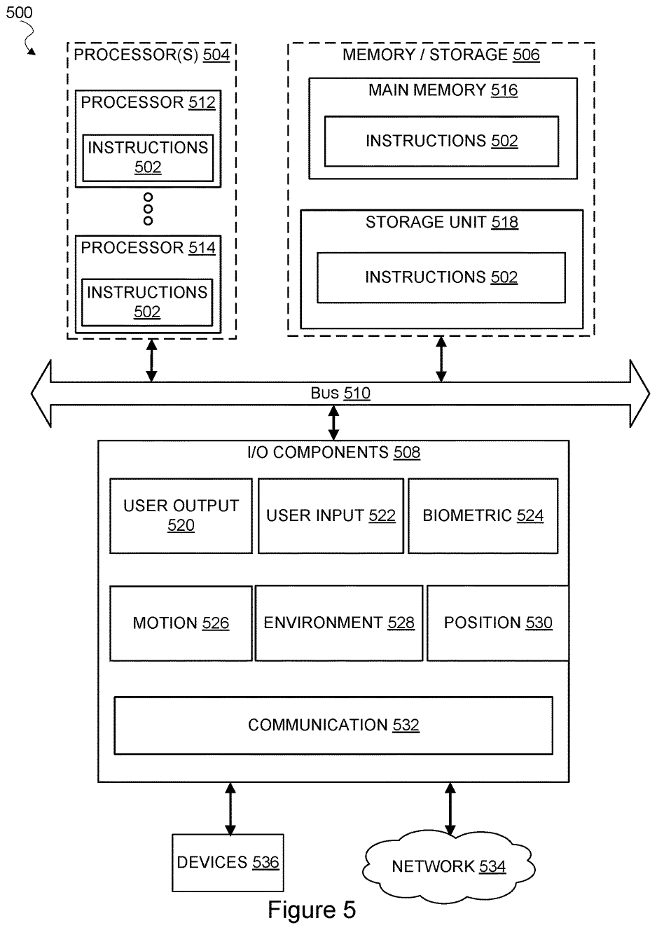

FIG. 5 is a block diagram illustrating components of a machine 500, according to some example implementations, able to read instructions from a machine-readable medium (e.g., a machine-readable storage medium) and perform any one or more of the methodologies discussed herein. Specifically, FIG. 5 shows a diagrammatic representation of the machine 500 in the example form of a computer system, within which instructions 502 (e.g., software, a program, an application, an applet, an app, or other executable code) for causing the machine 500 to perform any one or more of the methodologies discussed herein may be executed. As such, the instructions 502 may be used to implement modules or components described herein. The instructions 502 transform the general, non-programmed machine 500 into a particular machine 500 programmed to carry out the described and illustrated functions in the manner described. In alternative implementations, the machine 500 operates as a standalone device or may be coupled (e.g., networked) to other machines. In a networked deployment, the machine 500 may operate in the capacity of a server machine or a client machine in a server-client network environment, or as a peer machine in a peer-to-peer (or distributed) network environment. The machine 500 may comprise, but not be limited to, a server computer, a client computer, a personal computer (PC), a tablet computer, a laptop computer, a netbook, a set-top box (STB), a personal digital assistant (PDA), an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 502, sequentially or otherwise, that specify actions to be taken by machine 500. Further, while only a single machine 500 is illustrated, the term “machine” shall also be taken to include a collection of machines that individually or jointly execute the instructions 502 to perform any one or more of the methodologies discussed herein.

The machine 500 may include processors 504, memory/storage 506, and I/O components 508, which may be configured to communicate with each other such as via a bus 510. In an example implementation, the processors 504 (e.g., a central processing unit (CPU), a reduced instruction set computing (RISC) processor, a complex instruction set computing (CISC) processor, a graphics processing unit (GPU), a digital signal processor (DSP), an application-specific integrated circuit (ASIC), a radio-frequency integrated circuit (RFIC), another processor, or any suitable combination thereof) may include, for example, a processor 512 and a processor 514 that may execute the instructions 502. The term “processor” is intended to include multi-core processors 504 that may comprise two or more independent processors (sometimes referred to as “cores”) that may execute instructions 502 contemporaneously. Although FIG. 5 shows multiple processors 504, the machine 500 may include a single processor 512 with a single core, a single processor 512 with multiple cores (e.g., a multi-core processor), multiple processors 512, 514 with a single core, multiple processors 512, 514 with multiple cores, or any combination thereof.

The memory/storage 506 may include memory, such as a main memory 516, or other memory storage, and a storage unit 518, both accessible to the processors 504 such as via the bus 510. The storage unit 518 and main memory 516 store the instructions 502 embodying any one or more of the methodologies or functions described herein. The instructions 502 may also reside, completely or partially, within the main memory 516, within the storage unit 518, within at least one of the processors 504 (e.g., within the processor's cache memory), or any suitable combination thereof, during execution thereof by the machine 500. Accordingly, the main memory 516, the storage unit 518, and the memory of processors 504 are examples of machine-readable media.

The I/O components 508 may include a wide variety of components to receive input, provide output, produce output, transmit information, exchange information, capture measurements, and so on. The specific I/O components 508 that are included in a particular machine 500 will depend on the type of machine. For example, portable machines such as mobile phones will likely include a touch input device or other such input mechanisms, while a headless server machine will likely not include such a touch input device. It will be appreciated that the I/O components 508 may include many other components that are not shown in FIG. 5. The I/O components 508 are grouped according to functionality merely for simplifying the following discussion and the grouping is in no way limiting. In various example implementations, the I/O components 508 may include user output components 520 and user input components 522. The user output components 520 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The user input components 522 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or other pointing instrument), tactile input components (e.g., a physical button, a touch screen that provides location or force of touches or touch gestures, or other tactile input components), audio input components (e.g., a microphone), and the like.

In further example implementations, the I/O components 508 may include biometric components 524, motion components 526, environmental components 528, or position components 530 among a wide array of other components. For example, the biometric components 524 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 526 may include acceleration sensor components (e.g., accelerometer), gravitation sensor components, rotation sensor components (e.g., gyroscope), and so forth. The environmental components 528 may include, for example, illumination sensor components (e.g., photometer), temperature sensor components (e.g., one or more thermometer that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 530 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 508 may include communication components 532 operable to couple the machine 500 to a network 534 or devices 536. For example, the communication components 532 may include a network interface component or other suitable device to interface with the network 534. In further examples, communication components 532 may include wired communication components, wireless communication components, cellular communication components, near field communication (NFC) components, Bluetooth® components (e.g., Bluetooth® Low Energy), Wi-Fi® components, and other communication components to provide communication via other modalities. The devices 536 may be another machine 500 or any of a wide variety of peripheral devices (e.g., a peripheral device coupled via a USB).

Moreover, the communication components 532 may detect identifiers or include components operable to detect identifiers. For example, the communication components 532 may include radio frequency identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 532, such as location via Internet Protocol (IP) geo-location, location via Wi-Fi® signal triangulation, location via detecting an NFC beacon signal that may indicate a particular location, and so forth.

As used herein, “component” refers to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various example implementations, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein.

A hardware component may also be implemented mechanically, electronically, or any suitable combination thereof. For example, a hardware component may include dedicated circuitry or logic that is permanently configured to perform certain operations. A hardware component may be a special-purpose processor, such as a field-programmable gate array (FPGA) or an ASIC. A hardware component may also include programmable logic or circuitry that is temporarily configured by software to perform certain operations. For example, a hardware component may include software executed by a general-purpose processor 504 or other programmable processor. Once configured by such software, hardware components become specific machines (or specific components of a machine 500) uniquely tailored to perform the configured functions and are no longer general-purpose processors 504. It will be appreciated that the decision to implement a hardware component mechanically, in dedicated and permanently configured circuitry, or in temporarily configured circuitry (e.g., configured by software) may be driven by cost and time considerations. Accordingly, the phrase “hardware component” (or “hardware-implemented component”) should be understood to encompass a tangible entity, be that an entity that is physically constructed, permanently configured (e.g., hardwired), or temporarily configured (e.g., programmed) to operate in a certain manner or to perform certain operations described herein. Considering implementations in which hardware components are temporarily configured (e.g., programmed), each of the hardware components need not be configured or instantiated at any one instance in time. For example, where a hardware component comprises a general-purpose processor 504 configured by software to become a special-purpose processor, the general-purpose processor 504 may be configured as respectively different special-purpose processors (e.g., comprising different hardware components) at different times. Software accordingly configures a particular processor 512, 514 or processors 504, for example, to constitute a particular hardware component at one instance of time and to constitute a different hardware component at a different instance of time.

Hardware components can provide information to, and receive information from, other hardware components. Accordingly, the described hardware components may be regarded as being communicatively coupled. Where multiple hardware components exist contemporaneously, communications may be achieved through signal transmission (e.g., over appropriate circuits and buses) between or among two or more of the hardware components. In implementations in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output.

Hardware components may also initiate communications with input or output devices, and can operate on a resource (e.g., a collection of information). The various operations of example methods described herein may be performed, at least partially, by one or more processors 504 that are temporarily configured (e.g., by software) or permanently configured to perform the relevant operations. Whether temporarily or permanently configured, such processors 504 may constitute processor-implemented components that operate to perform one or more operations or functions described herein. As used herein, “processor-implemented component” refers to a hardware component implemented using one or more processors 504. Similarly, the methods described herein may be at least partially processor-implemented, with a particular processor 512, 514 or processors 504 being an example of hardware. For example, at least some of the operations of a method may be performed by one or more processors 504 or processor-implemented components. Moreover, the one or more processors 504 may also operate to support performance of the relevant operations in a “cloud computing” environment or as a “software as a service” (SaaS). For example, at least some of the operations may be performed by a group of computers (as examples of machines 500 including processors 504), with these operations being accessible via a network 534 (e.g., the Internet) and via one or more appropriate interfaces (e.g., an API). The performance of certain of the operations may be distributed among the processors, not only residing within a single machine 500, but deployed across a number of machines. In some example implementations, the processors 504 or processor-implemented components may be located in a single geographic location (e.g., within a home environment, an office environment, or a server farm). In other example implementations, the processors 504 or processor-implemented components may be distributed across a number of geographic locations.

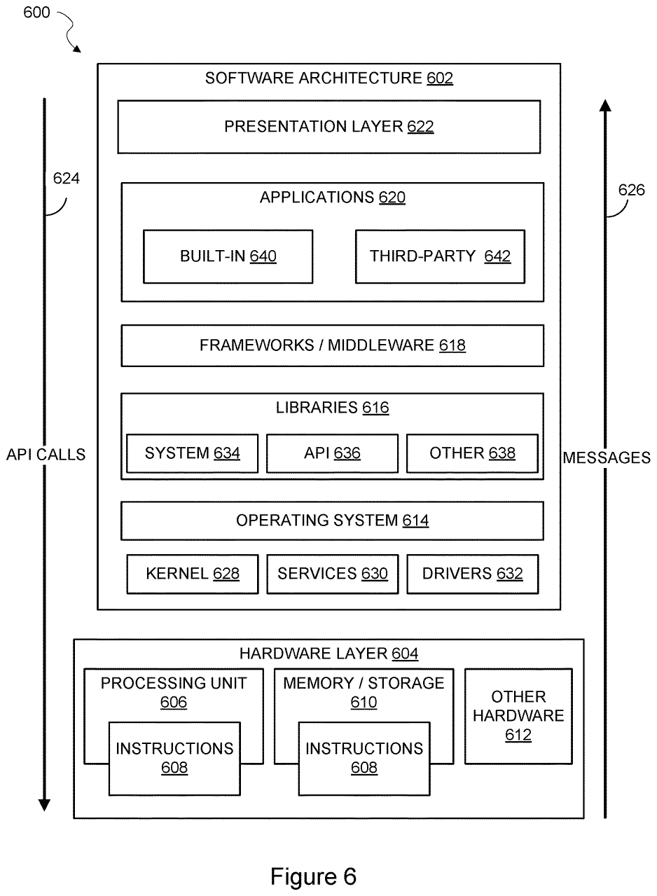

FIG. 6 is a block diagram illustrating system 600 that includes an example software architecture 602, which may be used in conjunction with various hardware architectures herein described. FIG. 6 is a non-limiting example of a software architecture, and it will be appreciated that many other architectures may be implemented to facilitate the functionality described herein. The software architecture 602 may execute on hardware such as machine 500 of FIG. 5 that includes, among other things, processors 504, memory/storage 506, and input/output (I/O) components 508. A representative hardware layer 604 is illustrated and can represent, for example, the machine 500 of FIG. 5. The representative hardware layer 604 includes a processing unit 606 having associated executable instructions 608. Executable instructions 608 represent the executable instructions of the software architecture 602, including implementation of the methods, components, and so forth described herein. The hardware layer 604 also includes at least one of memory or storage modules memory/storage 610, which also have executable instructions 608. The hardware layer 604 may also comprise other hardware 612.

In the example architecture of FIG. 6, the software architecture 602 may be conceptualized as a stack of layers where each layer provides particular functionality. For example, the software architecture 602 may include layers such as an operating system 614, libraries 616, frameworks/middleware 618, applications 620, and a presentation layer 622. Operationally, the applications 620 or other components within the layers may invoke API calls 624 through the software stack and receive messages 626 in response to the API calls 624. The layers illustrated are representative in nature and not all software architectures have all layers. For example, some mobile or special purpose operating systems may not provide a frameworks/middleware 618, while others may provide such a layer. Other software architectures may include additional or different layers.

The operating system 614 may manage hardware resources and provide common services. The operating system 614 may include, for example, a kernel 628, services 630, and drivers 632. The kernel 628 may act as an abstraction layer between the hardware and the other software layers. For example, the kernel 628 may be responsible for memory management, processor management (e.g., scheduling), component management, networking, security settings, and so on. The services 630 may provide other common services for the other software layers. The drivers 632 are responsible for controlling or interfacing with the underlying hardware. For instance, the drivers 632 include display drivers, camera drivers, Bluetooth® drivers, flash memory drivers, serial communication drivers (e.g., Universal Serial Bus (USB) drivers), Wi-Fi® drivers, audio drivers, power management drivers, and so forth depending on the hardware configuration.

The libraries 616 provide a common infrastructure that is used by at least one of the applications 620, other components, or layers. The libraries 616 provide functionality that allows other software components to perform tasks in an easier fashion than to interface directly with the underlying operating system 614 functionality (e.g., kernel 628, services 630, drivers 632). The libraries 616 may include system libraries 634 (e.g., C standard library) that may provide functions such as memory allocation functions, string manipulation functions, mathematical functions, and the like. In addition, the libraries 616 may include API libraries 636 such as media libraries (e.g., libraries to support presentation and manipulation of various media format such as MPEG4, H.264, MP3, AAC, AMR, JPG, PNG), graphics libraries (e.g., an OpenGL framework that may be used to render two-dimensional and three-dimensional in a graphic content on a display), database libraries (e.g., SQLite that may provide various relational database functions), web libraries (e.g., WebKit that may provide web browsing functionality), and the like. The libraries 616 may also include a wide variety of other libraries 638 to provide many other APIs to the applications 620 and other software components/modules.

The frameworks/middleware 618 (also sometimes referred to as middleware) provide a higher-level common infrastructure that may be used by the applications 620 or other software components/modules. For example, the frameworks/middleware 618 may provide various graphical user interface functions, high-level resource management, high-level location services, and so forth. The frameworks/middleware 618 may provide a broad spectrum of other APIs that may be utilized by the applications 620 or other software components/modules, some of which may be specific to a particular operating system 614 or platform.

The applications 620 include built-in applications 640 and third-party applications 642. Examples of representative built-in applications 640 may include, but are not limited to, a contacts application, a browser application, a book reader application, a location application, a media application, a messaging application, or a game application. Third-party applications 642 may include an application developed using the ANDROID™ or IOS™ software development kit (SDK) by an entity other than the vendor of the particular platform and may be mobile software running on a mobile operating system such as IOS™, ANDROID™, WINDOWS® Phone, or other mobile operating systems. The third-party applications 642 may invoke the API calls 624 provided by the mobile operating system (such as operating system 614) to facilitate functionality described herein.

The applications 620 may use built-in operating system functions (e.g., kernel 628, services 630, drivers 632), libraries 616, and frameworks/middleware 618 to create UIs to interact with users of the system. Alternatively, or additionally, in some systems, interactions with a user may occur through a presentation layer, such as presentation layer 622. In these systems, the application/component “logic” can be separated from the aspects of the application/component that interact with a user.

At least some of the processes described herein can be embodied in computer-readable instructions for execution by one or more processors such that the operations of the processes may be performed in part or in whole by the functional components of one or more computer systems. Accordingly, computer-implemented processes described herein are by way of example with reference thereto, in some situations. However, in other implementations, at least some of the operations of the computer-implemented processes described herein can be deployed on various other hardware configurations. The computer-implemented processes described herein are therefore not intended to be limited to the systems and configurations described with respect to FIGS. 5 and 6 and can be implemented in whole, or in part, by one or more additional system and/or components.

Although the flowcharts described herein can show operations as a sequential process, many of the operations can be performed in parallel or concurrently. In addition, the order of the operations can be re-arranged. A process is terminated when its operations are completed. A process can correspond to a method, a procedure, an algorithm, etc. The operations of methods may be performed in whole or in part, can be performed in conjunction with some or all of the operations in other methods, and can be performed by any number of different systems, such as the systems described herein, or any portion thereof, such as a processor included in any of the systems.

### EXAMPLE

A number of cancer samples was obtained from subjects in which colorectal cancer (CRC) was detected and a number of background samples was obtained from subject in which cancer was not detected. The first number of samples was obtained from individuals in different stages of CRC. MAF was also determined for a portion of the first number of samples. A direct methylation matching algorithm was applied using a set of 200 randomly selected DMRs in conjunction with a filter on the background samples. Counts of sequence representations corresponding to the cancer samples and the background samples are determined for each of the 200 DMRs.

Initially, a matching function was defined between any two fragments that indicated overlapping on at least a particular number of their CpGs (for example, at least 2 CpGs, at least 3 CpGs, at least 4 CpGs or at least 5 CpGs) and matching on at least particular percentage (e.g., >50%, >55%, >60%, >65%, >70%, >75%, >80%) of them, and do the following two filtering steps: (1) From a query plasma sample, remove all fragments that have any matching (by the above function) fragment in a normal background pool of fragments. This normal pool may contain data from several samples and (2) From this filtered set of fragments, either (i) take these as-is (no matching to tissue), or (ii) keep only fragments matching some molecule in the sample's paired tissue or keep only fragments matching some molecules an unpaired pool of tissue. In this case, all other tissue samples were used aside from the plasma sample's pair.

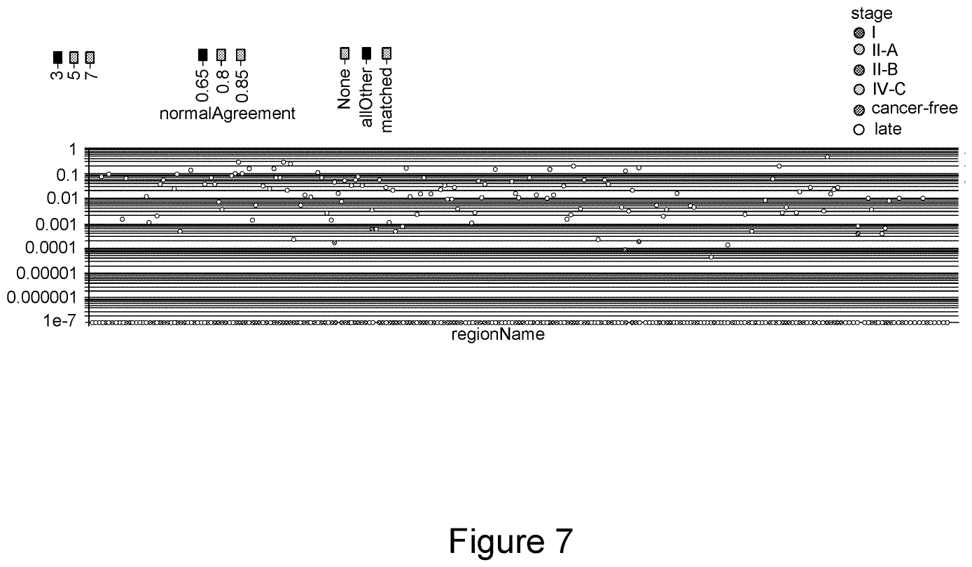

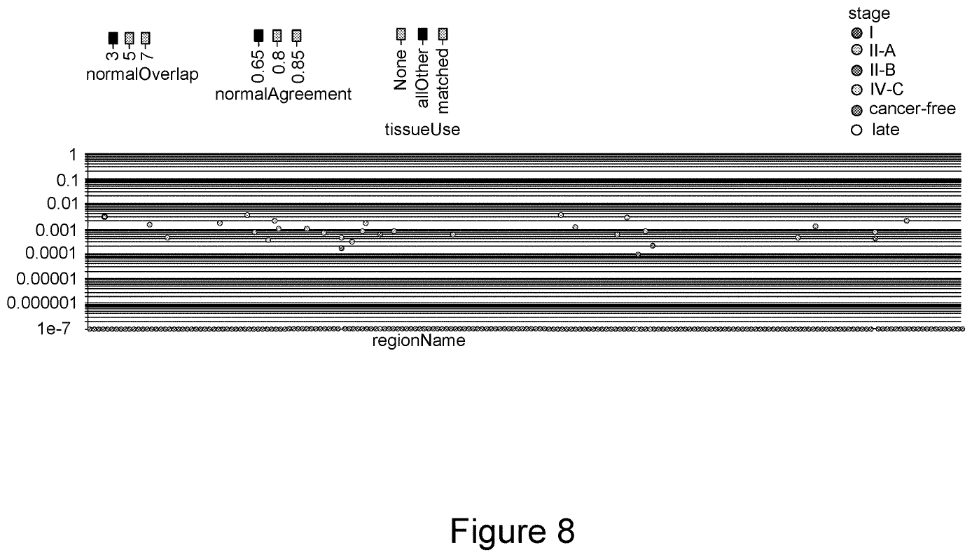

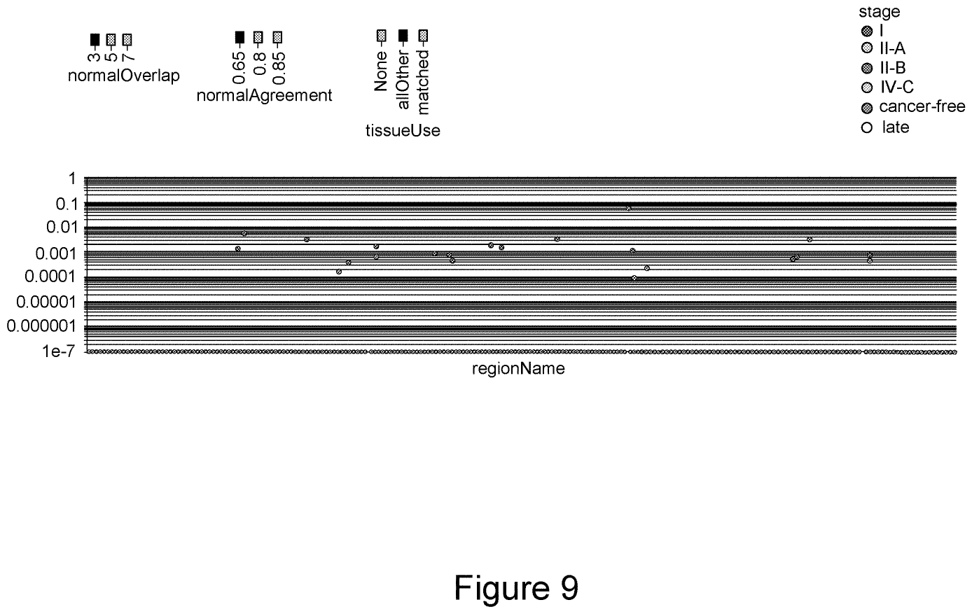

The data was analyzed to determine a pool size for the background molecules to be used in a paired dataset analysis with the tissue samples. A normal pool of a particular size was fixed and other parameters were varied: Overlap to define a match was set at 3, 5, 7 and agreement on this overlap was set at 0.65 (⅔), 0.8 (⅘) and 0.85 ( 6/7). After experimentation, a parameter setting of a particular number of CpGs overlap and ⅔ matches showed that the normal noise is down to zero for most regions. Using these parameter settings, quantitative measures were determined for individual regions of the 200 regions for background samples and cancer samples in relation to different cancer stages. FIG. 7 is a graphical representation showing a number of nucleic acid molecules that correspond to a number of genomic regions and that satisfy the parameter setting for samples corresponding to subjects in which late-stage cancer is present. In addition, FIG. 8 is a graphical representation showing a number of nucleic acid molecules that correspond to a number of genomic regions and that satisfy the parameter setting for samples corresponding to subjects in which stage II cancer is present. Further, FIG. 9 is a graphical representation showing a number of nucleic acid molecules that correspond to a number of genomic regions and that satisfy the parameter setting for samples corresponding to subject in which stage I cancer is present.

What is claimed is:

1. A method comprising:
accessing one or more data sets indicating (i) first methylation states of individual cytosine nucleotides included in first cytosine-guanine dinucleotides (CpGs) of first deoxyribonucleic acid (DNA) molecule sequence representations in a first dataset derived from first samples obtained from first subjects in which a tumor is not detected and (ii) second methylation states of individual cytosine nucleotides included in second CpGs of second DNA molecule sequence representations in a second dataset derived from one or more second samples obtained from a second subject;
analyzing the second methylation states of the second CpGs with respect to the first methylation states of the first CpGs to determine a subset of the second DNA molecule sequence representations, individual second DNA molecule sequence representations of the subset of the second DNA molecule sequence representations having at least a threshold amount of the second CpGs that correspond to a number of the first CpGs;
determining one or more quantitative measures with respect to the subset of the second DNA molecule sequence representations; and
determining an indication of cancer being present in the second subject based on the one or more quantitative measures.

2. The method of claim 1, further comprising:
accessing an additional data set indicating third methylation states of individual cytosine nucleotides included in third CpGs of third DNA molecule sequence representations in a third dataset derived from one or more third samples obtained from one or more third subjects in which a tumor is detected, the one or more third samples including one or more tissue samples obtained from the one or more third subjects; and
analyzing the second methylation states of the second CpGs with respect to the third methylation states of the third CpGs to determine an additional subset of the second DNA molecule sequence representations, additional individual second DNA molecule sequence representations of the additional subset of the second DNA molecule sequence representations having at least the threshold amount of the second CpGs that correspond to the third CpGs.

3. The method of claim 2, comprising:
determining a group of the second DNA molecule sequence representations that excludes the subset of the second DNA molecule sequence representations and includes the additional subset of the second DNA molecule sequence representations.

4. The method of claim 3, wherein determining the subset of the second DNA molecule sequence representations comprises:
determining an individual second DNA molecule sequence representation having a plurality of CpGs with genomic locations that align with a plurality of additional CpGs at the genomic locations of an individual first DNA molecule sequence representation;
determining that a number of the plurality of CpGs of the individual second DNA molecule sequence representation having methylation states of individual cytosines that are the same as additional methylation states of individual additional cytosines of the plurality of additional CpGs of the individual first DNA molecule sequence representation is at least a threshold number; and
determining that the individual second DNA molecule sequence representation is to be excluded from the group of the second DNA molecule sequence representations.

5. The method of claim 3, wherein determining the group of the second DNA molecule sequence representations that excludes the subset of the second DNA molecule sequence representations and includes the additional subset of the second DNA molecule sequence representations comprises:
determining an individual second DNA molecule sequence representation having a plurality of CpGs with genomic locations that align with a plurality of additional CpGs at the genomic locations of an individual first DNA molecule sequence representation;
determining that a number of the plurality of CpGs of the individual second DNA molecule sequence representation having methylation states of individual cytosines that are different from additional methylation states of individual additional cytosines of the plurality of additional CpGs of an individual third DNA molecule sequence representation is at least a threshold number; and
determining that the individual second DNA molecule sequence representation is to be included in an additional group of DNA molecule sequence representations that is analyzed with respect to the additional data set.

6. The method of claim 5, comprising:
determining that the individual second DNA molecule sequence representation has a plurality of CpGs with genomic locations that align with a plurality of additional CpGs at the genomic locations of the individual third DNA molecule sequence representation;
determining that a number of the plurality of CpGs of the individual second DNA molecule sequence representation have methylation states of individual cytosines that are the same as additional methylation states of individual additional cytosines of the plurality of additional CpGs of the individual third DNA molecule sequence representation is at least a threshold number; and
determining that the individual second DNA molecule sequence representation is to be included in the group of the second DNA molecule sequence representations.

7. The method of claim 6, comprising:
determining that a number of first DNA molecule sequence representations are matched with an individual second DNA molecule sequence representation and if that number is greater than a threshold then that individual second DNA molecule sequence representation is excluded from the subset of the second DNA molecule sequence representations.

8. The method of claim 6, wherein determining that the individual second DNA molecule sequence representation is to be included in the group of the second DNA molecule sequence representations comprises:
determining that a number of the third DNA molecule sequence representations are matched with an individual second molecule sequence representation and if that number is greater than a threshold then that individual second DNA molecule sequence representation is included in the group of the second DNA molecule sequence representations.

9. The method of claim 5, wherein determining that the individual second DNA molecule sequence representation has a plurality of CpGs with genomic locations that align with the plurality of additional CpGs at the genomic locations of the individual first DNA molecule sequence representation comprises:
analyzing individual positions of individual CpGs of the individual second DNA molecule sequence representation with respect to individual additional positions of a plurality of additional CpGs of a plurality of first DNA molecule sequence representations to determine one or more DNA molecule sequence representation pairings, wherein an individual DNA molecule sequence representations pairing includes the second DNA molecule sequence representations and a first DNA molecule sequence representation having at least a threshold number of aligned CpGs, wherein an aligned CpG corresponds to a position of a reference genome where (i) a CpG of the individual second DNA molecule sequence representation is located and (ii) a CpG of the first DNA molecule sequence representation is located.

10. The method of claim 9, wherein analyzing the second methylation states of the second CpGs with respect to the first methylation states of the first CpGs to determine the subset of the second DNA molecule sequence representations comprises:
determining that a methylation state of a CpG of the individual second molecule sequence representation corresponds to an additional methylation state of an additional CpG of a respective first DNA molecule sequence representation for at least a threshold number of aligned CpGs of the individual second molecule sequence representation and the respective first DNA molecule sequence representation.

11. The method of claim 6, wherein determining that the individual second DNA molecule sequence representation has a plurality of CpGs with genomic locations that align with the plurality of additional CpGs at the genomic locations of the individual third DNA molecule sequence representation comprises:
analyzing individual positions of individual CpGs of the individual second DNA molecule sequence representation with respect to individual additional positions of a plurality of additional CpGs of a plurality of the third DNA molecule sequence representations to determine one or more DNA molecule sequence representation pairings, wherein an individual DNA molecule sequence representation pairing includes the individual second DNA molecule sequence representation and a third DNA molecule sequence representation having at least a threshold number of aligned CpGs, wherein an aligned CpG corresponds to a position of a reference genome where (i) a CpG of the individual second DNA molecule sequence representation is located and (ii) a CpG of the first DNA molecule sequence representation is located.

12. The method of claim 11, wherein analyzing the second methylation states of the second CpGs with respect to the third methylation states of the third CpGs to determine the additional subset of the individual second DNA molecule sequence representation comprises:
determining that a methylation state of a CpG of the individual second DNA molecule sequence representation corresponds to an additional methylation state of an additional CpG of a respective third DNA molecule sequence representation for at least at threshold number of aligned CpGs of the individual second DNA molecule sequence representation and the respective third DNA molecule sequence representation.

13. The method of any one of claims 1-12, wherein the first DNA molecule sequence representations correspond to at least one of:
cell-free DNA molecules derived from one or more plasma samples obtained from the first subjects; or
DNA molecules obtained from tissue samples extracted from the first subjects.

14. The method of any one of claims 1-13, wherein the second DNA molecule sequence representations correspond to at least one of:
cell-free DNA molecules derived from one or more plasma samples obtained from the second subject; or
DNA molecules obtained from a tissue sample extracted from the second subject.

15. The method of any one of claims 2-12, wherein the third DNA molecule sequence representations correspond to at least one of:
cell-free DNA molecules derived from one or more plasma samples obtained from the one or more third subjects; or
DNA molecules obtained from tissue samples extracted from the one or more third subjects.

16. The method of any one of claims 3-15, comprising:
determining a number of the second DNA molecule sequence representations included in the group of the second DNA molecule sequence representations; and
determining an aggregate number of second DNA molecule sequence representations included in the second dataset;
wherein the one or more quantitative measures correspond to a proportion of the number of second DNA molecule sequence representations in relation to the aggregate number of second DNA molecule sequence representations.

17. The method of any one of claims 3-15, wherein the one or more quantitative measures include counts of the second DNA molecule sequence representations included in the group of the second DNA molecule sequence representations.

18. The method of any one of claims 2-12, wherein a third subject of the one or more third subjects includes the second subject.

19. The method of any one of claims 2-12, wherein a type of cancer is present in the one or more third subjects and the indication of cancer being present in the second subject indicates that the type of cancer is present in the second subject.

20. The method of claim 19, wherein a same type of cancer is present in the one or more third subjects.

21. The method of claim 19, comprising:
accessing a further data set indicating fourth methylation states of individual cytosine nucleotides included in fourth CpGs of fourth DNA molecule sequence representations derived from one or more fourth samples obtained from one or more fourth subjects in which a tumor corresponding to an additional type of cancer is detected;
analyzing the second methylation states of the second CpGs with respect to the fourth methylation states of the fourth CpGs to determine a further subset of the second DNA molecule sequence representations, further individual second DNA molecule sequence representations of the further subset of the second DNA molecule sequence representations having at least the threshold amount of the second CpGs that correspond to the fourth CpGs;
determining one or more additional quantitative measures with respect to the subset of the second DNA molecule sequence representations and the further subset of the second DNA molecule sequence representations; and
determining an additional indication of the additional type of cancer being present in the second subject based on the one or more additional quantitative measures.

22. The method of claim 21, wherein the one or more fourth samples include at least one of:
cell-free DNA molecules derived from one or more plasma samples obtained from the one or more fourth subjects; or
DNA molecules obtained from tissue samples extracted from the one or more fourth subjects.

23. The method of any one of claims 1-22, wherein the indication of cancer being present in the second subject corresponds to tumor fraction, and the method comprises:
analyzing the tumor fraction to determine a stage of cancer present in the second subject.

24. The method of any one of claims 2-23, wherein:
at least a portion of the first methylation states indicate first methylated cytosines of the first CpGs;
at least a portion of the second methylation states indicate second methylated cytosines of the second CpGs; and
at least a portion of the third methylation states indicate third methylated cytosines of the third CpGs.

25. The method of any one of claims 2-23, wherein:
at least a portion of the first methylation states indicate first unmethylated cytosines of the first CpGs;
at least a portion of the second methylation states indicate second unmethylated cytosines of the second CpGs; and
at least a portion of the third methylation states indicate third unmethylated cytosines of the third CpGs.

26. The method of any one of claims 2-25, wherein at least one of the first CpGs, the second CpGs, or the third CpGs are located in a plurality of classification regions, the plurality of classification regions including CpGs having a first methylation state in subjects in which cancer is present and a second methylation state different from the first methylation state in additional subjects in which cancer is not present.

27. The method of claim 26, comprising:
determining that at least a threshold number of at least one of the first DNA molecule sequence representations, the second DNA molecule sequence representations, or the third DNA molecule sequence representations correspond to a subset of the plurality of classification regions;
wherein at least one of the first CpGs, the second CpGs, or the third CpGs are located in the subset of the plurality of classification regions.

28. The method of any one of claims 3-27, wherein determining that the group of the second DNA molecule sequence representations includes the additional subset of the second DNA molecule sequence representations comprises:
determining that a number of the additional subset of the second DNA molecule sequence representations is at least an additional threshold number.

29. The method of any one of claims 1-28, wherein:
the one or more datasets include a first additional dataset indicating first additional methylation states of individual cytosine nucleotides included in first additional CpGs of first additional DNA molecule sequence representations derived from one or more buffy coat samples obtained from the second subject;
wherein the second methylation states of the second CpGs are analyzed with respect to the first additional methylation states of the first additional CpGs to determine the subset of the second DNA molecule sequence representations.

30. The method of claim 29, comprising:
determining that the second methylation states of a first additional group of the second DNA molecule sequence representations corresponds to the first additional methylation states of at least a portion of the first additional DNA molecule sequence representations; and
determining that the first additional group of the second DNA molecule sequence representations is excluded from the subset of the second DNA molecule sequence representations.

31. The method of claim 30, wherein determining the subset of the second DNA molecule sequence representations comprises:
determining an individual second DNA molecule sequence representation having a plurality of CpGs with genomic locations that align with a plurality of the first additional CpGs at the genomic locations of an individual first additional DNA molecule sequence representation;
determining that a number of the plurality of CpGs of the individual second DNA molecule sequence representation having methylation states of individual cytosines that are the same as the first additional methylation states of individual first additional cytosines of the plurality of first additional CpGs of the individual first additional DNA molecule sequence representation is at least a threshold number; and
determining that the individual second DNA molecule sequence representation is to be excluded from the subset of the second DNA molecule sequence representations.

32. The method of any one of claims 1-31, wherein:
the one or more datasets include a second additional dataset indicating second additional methylation states of individual cytosine nucleotides included in second additional CpGs of second additional DNA molecule sequence representations derived from one or more buffy coat samples obtained from at least a portion of the first subjects;
wherein the second methylation states of the second CpGs are analyzed with respect to the second additional methylation states of the first additional CpGs to determine the subset of the second DNA molecule sequence representations.

33. The method of claim 32, comprising:
determining that the second methylation states of a second additional group of the second DNA molecule sequence representations corresponds to the second additional methylation states of at least a portion of the second additional DNA molecule sequence representations; and
determining that the second additional group of the second DNA molecule sequence representations is excluded from the subset of the second DNA molecule sequence representations.

34. The method of claim 33, wherein determining the subset of the second DNA molecule sequence representations comprises:
determining an individual second DNA molecule sequence representation having a plurality of CpGs with genomic locations that align with a plurality of the second additional CpGs at the genomic locations of an individual second additional DNA molecule sequence representation;
determining that a number of the plurality of CpGs of the individual second DNA molecule sequence representation having methylation states of individual cytosines that are the same as the second additional methylation states of individual second additional cytosines of the plurality of second additional CpGs of the individual second additional DNA molecule sequence representation is at least a threshold number; and
determining that the individual second DNA molecule sequence representation is to be excluded from the subset of the second DNA molecule sequence representations.
